(19) United States
(12) Patent Application Publication
(10) Pub. No.: US 2026/0279586 A1
(43) Pub. Date: Sep. 17, 2026

(54) SYSTEMS AND METHODS OF ARTIFICIAL INTELLIGENCE-BASED SEPSIS PREDICTION

(71) Applicant: PRENOSIS, INC., Chicago, IL (US)

(72) Inventors: Akhil Bhargava, Chicago, IL (US); Jonah Weiner Ellman, Chicago, IL (US); Lee Alan Schmalz, Chicago, IL (US); Gregory Luke Watson, Vista, CA (US); Carlos German López-Espina, Evanston, IL (US); Bobby Reddy, Jr., Chicago, IL (US)

(21) Appl. No.: 19/471,933

(22) PCT Filed: Apr. 12, 2024
(86) PCT No.: PCT/US2024/024461

§ 371 Date: Oct. 2, 2025

Related U.S. Application Data
(60) Provisional application No. 63/459,568, filed on Apr. 14, 2023.

(51) Int. Cl. G16H 50/30; G16H 20/10; G16H 50/20; G16H 50/70

(52) U.S. Cl. CPC G16H 50/30; G16H 20/10; G16H 50/20; G16H 50/70

(57) ABSTRACT

The present disclosure encompasses systems and methods for determining the presence of sepsis in an individual or the risk of developing sepsis in an individual at risk therefore, including within about 24 hours of acquiring measurements and/or information of one or more factors associated with the individual. In specific embodiments, one or more input features, such as one or more demographic, patient assessment, vital sign, hematological laboratory measurement, chemistry panel measurement, and/or sepsis-associated biomarkers may be utilized in generating an output, such as a quantitative value, that indicates whether or not an individual is at low risk, medium risk, high risk, or very high risk for the onset of sepsis.

### CROSS-REFERENCE TO RELATED APPLICATION

This application claims priority and the benefit of U.S. Provisional Patent Application No. 63/459,568, filed on Apr. 14, 2023, the contents of which are hereby incorporated by reference in their entirety.

### FIELD

The present disclosure generally relates at least to methods, compositions, and systems concerning medicine, physiology, and biology. More particularly, the present disclosure relates to risk assessment for onset of sepsis.

### BACKGROUND

In many diseases and conditions, a positive outcome of treatment and/or prophylaxis is strongly correlated with early and/or accurate diagnosis of the disease or condition. However, often there are no effective methods of early diagnosis and treatments that are therefore often administered too late, inappropriately, or to individuals who will not benefit from it.

Sepsis can be a serious situation, an often life-threatening disease calling for urgent and comprehensive care. Treatment depends on the type of infection, but usually begins with antibiotics or similar medications. As sepsis may be the result of infection by a wide variety of organisms, it is a condition that is particularly difficult to predict and diagnose early enough for effective intervention. It is an excessive and uncontrolled inflammatory response in an individual usually resulting from an individual's inappropriate immune system response to a pathogenic organism. Moreover, there may not be significant numbers of organisms at accessible sites or in body fluids of the affected individual, thus increasing the difficulty of diagnosis. There is therefore a need to identify biomarkers indicating the risk, or early onset of sepsis, regardless of the causative agent, to allow early and effective intervention. Differentiating between patients who are at risk of developing sepsis and those who are not, will also assist in managing the disease condition. There is therefore an immediate need for the identification of biomarkers that are measurable and specific for the condition, and indicative of the risk of progression to, or early onset of, sepsis as well as methods for using said markers in screening.

### SUMMARY

Embodiments of the disclosure include methods of generating an indicator of risk for sepsis, the method comprising receiving at least one input feature corresponding to a patient; analyzing the at least one input feature using a machine learning model to generate a predicted score that indicates a probability of the patient having or developing sepsis within a predetermined time-span based on the at least one input feature; wherein the at least one input feature is associated with sepsis.

In some embodiments, there is a method for providing a sepsis diagnosis, the method comprising receiving data corresponding to a patient, the data comprising a concentration of one or more input features; analyzing the data using at least one machine learning model to generate a predicted score that indicates a probability of the patient having or developing sepsis within a predetermined time-span based on at least one input feature selected from a group of input features listed in any one of Tables 8-13, wherein the group of input features listed in Tabless 8-13 is associated with sepsis, and wherein the group of input features is listed in Table 8-13 with respect to relative significance to the predicted score; and generating a diagnosis output based on the predicted score. In specific embodiments, generating the diagnosis output comprises determining that the predicted score falls within one of four categories; and generating, based on the category in which the predicted score falls, the diagnosis output that includes that the patient has a low, medium, high, or very high probability of having or developing sepsis within the predetermined time-span. In some embodiments, the predicted score ranges between 0 and 1. In some embodiments, the four categories comprise a low category, a medium category, a high category, and a very high category, and wherein the low category is designated as having the predicted score below a first threshold, the medium category is designated as having the predicted score between the first threshold and a second threshold, the high category is designated as having the predicted score between the second threshold and a third threshold, and the very high category is designated as having the predicted score above the third threshold.

In particular embodiments, there is a method for generating a sepsis diagnosis, the method comprising: receiving input features corresponding to a patient; analyzing the input features using a machine learning model to generate a predicted score that indicates a probability of the patient having or developing sepsis within a predetermined time-span based on at least one input feature selected from a group of input features listed in any one of Tables 8-13, wherein the group of input features listed in any one of Tables 8-13 is associated with sepsis, and wherein the group of input features are listed in Tables 8-13, in no particular order of significance. Furthermore, the group of input features listed in Tables 8-13 can be considered to be listed with respect to relative significance to the predicted score. Any method may generate a diagnosis output based on the predicted score. In certain embodiments, the input features comprise at least one clinical parameter and at least one biomarker listed in the group of input features listed in any one of Tables 8-13.

### BRIEF DESCRIPTION OF THE DRAWINGS

For a more complete understanding of the principles disclosed herein, and the advantages thereof, reference is now made to the following descriptions taken in conjunction with the accompanying drawings, in which:

FIG. 1 is a depiction of the inputs, functions, artifacts, intermediate outputs, and final outputs used and generated in an example of a Sepsis ImmunoScore Algorithm. Functions are represented as Trigger Logic, Impute Input Features, Generate Pre-calibrated Score Function, Generate SHAP Values, Calibrate Predictions, and Generate Risk Category. Intermediate Data is reflected as Input Features (Non-Imputed) and pre-calibrated Sepsis Immunoscore. Artifacts are reflected in the cube-shaped steps. Final Output is represented as Input Features, Feature SHAP Values, Sepsis ImmunoScore, and Sepsis ImmunoScore Risk Category.

FIG. 2 is an example method for generating an indicator of risk for sepsis, in accordance with various embodiments.

FIG. 3A provides one example of SHAP values for a first individual utilizing Model 1A. FIG. 3B provides one example of SHAP values for a second individual utilizing Model 1A. FIG. 3C provides one example of SHAP values for a third individual utilizing Model 1A. In all cases, a value that increases risk of sepsis are the bars to the right, and a value that decreases the risk of sepsis are the bars to the left.

FIG. 4A provides one example of SHAP values for a first individual utilizing Model 2. FIG. 4B provides one example of SHAP values for a second individual utilizing Model 2. FIG. 4C provides one example of SHAP values for a third individual utilizing Model 2. In all cases, a value that increases risk of sepsis are the bars to the right, and a value that decreases the risk of sepsis are the bars to the left.

FIG. 5A provides one example of SHAP values for a first individual utilizing Model 3. FIG. 5B provides one example of SHAP values for a second individual utilizing Model 3. FIG. 5C provides one example of SHAP values for a third individual utilizing Model 3. In all cases, a value that increases risk of sepsis are the bars to the right, and a value that decreases the risk of sepsis are the bars to the left.

FIG. 6A provides one example of SHAP values for a first individual utilizing Model 4. FIG. 6B provides one example of SHAP values for a second individual utilizing Model 4. FIG. 6C provides one example of SHAP values for a third individual utilizing Model 4. In all cases, a value that increases risk of sepsis are the bars to the right, and a value that decreases the risk of sepsis are the bars to the left.

FIG. 7A provides one example of SHAP values for a first individual utilizing Model 5. FIG. 7B provides one example of SHAP values for a second individual utilizing Model 5. FIG. 7C provides one example of SHAP values for a third individual utilizing Model 5. In all cases, a value that increases risk of sepsis are the bars to the right, and a value that decreases the risk of sepsis are the bars to the left.

FIG. 8 is a block diagram of a computer system, in accordance with various embodiments.

FIG. 9A illustrates a non-limiting example of a 2-group, or bin, feature applied to Model 1A, in accordance with various embodiments.

FIG. 9B illustrates a non-limiting example of a 3-group, or bin, feature applied to Model 1A, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

### I. Exemplary Descriptions of Terms

As used herein the specification, “a” or “an” may mean one or more. As used herein in the claim(s), when used in conjunction with the word “comprising,” the words “a” or “an” may mean one or more than one. Some embodiments of the disclosure may consist of or consist essentially of one or more elements, method steps, and/or methods of the disclosure. It is contemplated that any method or composition described herein can be implemented with respect to any other method or composition described herein and that different embodiments may be combined.

The use of the term “or” in the claims is used to mean “and/or” unless explicitly indicated to refer to alternatives only or the alternatives are mutually exclusive, although the disclosure supports a definition that refers to only alternatives and “and/or.” For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment. As used herein “another” may mean at least a second or more.

The term “ones” means more than one.

As used herein, the term “plurality” may be 2, 3, 4, 5, 6, 7, 8, 9, 10, or more.

As used herein, the term “set of” means one or more. For example, a set of items includes one or more items.

As used herein, the phrase “at least one of,” when used with a list of items, means different combinations of one or more of the listed items may be used and only one of the items in the list may be needed. The item may be a particular object, thing, step, operation, process, or category. In other words, “at least one of” means any combination of items or number of items may be used from the list, but not all of the items in the list may be required. For example, without limitation, “at least one of item A, item B, or item C” means item A; item A and item B; item B; item A, item B, and item C; item B and item C; or item A and C. In some cases, “at least one of item A, item B, or item C” means, but is not limited to, two of item A, one of item B, and ten of item C; four of item B and seven of item C; or some other suitable combination.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in various embodiments.

The term “Artifact” as used herein refers to digital products that are used in an algorithm.

As used herein, an “artificial neural network” or “neural network” (NN) may refer to mathematical algorithms or computational models that mimic an interconnected group of artificial nodes or neurons that processes information based on a connectionistic approach to computation. Neural networks, which may also be referred to as neural nets, can employ one or more layers of nonlinear units to predict an output for a received input. Some neural networks include one or more hidden layers in addition to an output layer. The output of each hidden layer is used as input to the next layer in the network, i.e., the next hidden layer or the output layer. Each layer of the network generates an output from a received input in accordance with current values of a respective set of parameters. In the various embodiments, a reference to a “neural network” may be a reference to one or more neural networks.

A neural network may process information in two ways: when it is being trained it is in training mode and when it puts what it has learned into practice it is in inference (or prediction) mode. Neural networks learn through a feedback process (e.g., backpropagation) which allows the network to adjust the weight factors (modifying its behavior) of the individual nodes in the intermediate hidden layers so that the output matches the outputs of the training data. In other words, a neural network learns by being fed training data (learning examples) and eventually learns how to reach the correct output, even when it is presented with a new range or set of inputs. A neural network may include, for example, without limitation, at least one of a Feedforward Neural Network (FNN), a Recurrent Neural Network (RNN), a Modular Neural Network (MNN), a Convolutional Neural Network (CNN), a Residual Neural Network (ResNet), an Ordinary Differential Equations Neural Networks (neural-ODE), or another type of neural network.

The terms “adjudicate,” “adjudication,”, or adjudicating” as used herein refers to a physician adjudication process by which one or more physician reviews a patient case, typically via chart review, to make a retrospective determination of what happened.

The term “biomarker,” as used herein, generally refers to any measurable substance taken as a sample from a subject whose presence is indicative of some phenomenon. Non-limiting examples of such phenomenon can include an age of a subject, a disease state, a condition, or exposure to a compound or environmental condition. In various embodiments described herein, biomarkers included in analyses that also employ the age-related biomarkers may be used for diagnostic and/or therapeutic purposes (e.g., to diagnose a health state, a disease state). The term “biomarker” can be used interchangeably with the term “marker.”

The term “patient,” as used herein, generally refers to a mammalian subject. The mammal can be a human, or an animal including, but not limited to an equine, porcine, canine, feline, ungulate, and primate animal. In one embodiment, the individual is a human. The methods and uses described herein are useful for both medical and veterinary uses. A “patient” is a human subject unless specified to the contrary.

The term “training data,” as used herein generally refers to data that can be input into models, statistical models, algorithms and any system or process able to use existing data to make predictions.

As used herein, a “model” may include one or more algorithms, one or more mathematical techniques, one or more machine learning algorithms, or a combination thereof.

As used herein, “machine learning” may be the practice of using algorithms to parse data, learn from it, and then make a determination or prediction about something in the world. Machine learning uses algorithms that can learn from data without relying on rules-based programming. A machine learning algorithm may include a parametric model, a nonparametric model, a deep learning model, a neural network, a linear discriminant analysis model, a quadratic discriminant analysis model, a support vector machine, a random forest algorithm, a nearest neighbor algorithm, a combined discriminant analysis model, a k-means clustering algorithm, a supervised model, an unsupervised model, logistic regression model, a multivariable regression model, a penalized multivariable regression model, or another type of model.

The term “LOINC” as used herein refers to Logical Observation Identifiers, Names, and Codes (LOINC) is a standard that facilitates the exchange and pooling of results such as laboratory tests or vital signs for clinical care, outcomes management, and research.

The term “Sepsis ImmunoScore” as used herein refers to a probability of a patient having or developing sepsis, as defined by Sepsis-3, within 24 hours from the order time of the Sepsis ImmunoScore, in some embodiments.

The term “Sepsis ImmunoScore Algorithm” as used herein refers to a process of using a set of a patient's live-stream measured values to generate the Sepsis ImmunoScore.

The term “SHAP Value” as used herein refers to Shapley value that is the value associated with each input feature contribution to final prediction.

### II. Overview of Embodiments

Embodiments of the disclosure include generation of an indicator or output of any kind, such as a quantitative output (e.g., a score) or a qualitative output, that reflects an individual having sepsis or at risk of developing sepsis (such as a risk over the general population), including at least some cases developing sepsis within a specific time period. The sepsis may be of any kind, including sepsis, severe sepsis, or septic shock. In a specific embodiment, the output may or may not be live-streamed. In some embodiments, the individual has an infection as defined by presence of one of the following criteria:
1. Four or more Qualifying Antimicrobial Days (QAD) as defined by the Centers for Disease Control and Prevention (CDC) criteria.
2. A positive microbiology test or culture except for known contaminants (known contaminants within urine culture may be defined as “Lactobacilli, Corynebacteria species, Gardnerella, alphahaemolytic streptococci, and aerobes.)

The QAD rule follows the description in the CDC's Hospital Tool Kit for Adult Sepsis rules for infection (CDC Hospital Tool Kit for Adult Sepsis https://www.cdc.gov/sepsis/pdfs/Sepsis-Surveillance-Toolkit-Mar-2018_508.pdf).

In particular embodiments, methods and compositions of the disclosure concern the probability or risk of a patient having or developing sepsis, as defined by Sepsis-3 (Singer et al., JAMA. 2016 Feb. 23; 315(8): 801-810, which is incorporated by reference herein in its entirety), within 1, 2, 3, 4, 5, 6, 7, 8, 9, 10, 11, 12, 13, 14, 15, 16, 17, 18, 19, 20, 21, 22, 23, 24, or more hours from the time of patient assessment. The output of the patient analysis may be referred to herein as the Sepsis ImmunoScore.

The output of the methods may or may not be a continuous value, such as one between 0 and 100. The output may be displayed to users of a Software as a Medical device (SaMD), in at least some cases. In some embodiments, the output may be interpreted as the probability of a patient having or developing sepsis, as defined by Sepsis-3, within 24 hours from the order time of the Sepsis ImmunoScore. The Sepsis ImmunoScore may differ from the Uncalibrated Sepsis ImmunoScore prediction by imposing a calibration that refines the interpretability of the Uncalibrated Sepsis ImmunoScore prediction, in some embodiments.

In various embodiments, an individual is in need of a sepsis diagnosis or determination of a risk for sepsis. The individual may be of any age, but in specific embodiments, the individual is at least about 65 years of age or less than about 1 year of age. The individual may be hospitalized or recently hospitalized. The individual may or may not be immunocompromised. The individual may have an infection or at risk of having an infection, including bacterial, viral, fungal, prion, or parasitic, in certain embodiments. The individual may or may not have a chronic disease, such as heart disease, lung disease, cancer, kidney disease, hepatitis, and/or diabetes, merely as examples. In specific embodiments, the individual has infection of SARS-CoV2, influenza, meningitis, pneumonia, tuberculosis, E. coli, C. difficile, and so forth.

The individual may have one or more symptoms of sepsis, including high heart rate or weak pulse; fever, shivering, or feeling very cold; confusion or disorientation; shortness of breath; extreme pain or discomfort; and/or clammy or sweaty skin.

The individual may reside temporarily or permanently in a facility with others, such as a nursing home, rehabilitation center, skilled nursing facility, hospital, and so forth.

In specific embodiments, the individual is an adult 18 years old or older, with indicated suspicion of serious infection, as defined by the first order of a blood culture in an emergency room or Hospital environment.

In specific embodiments, methods and systems of the disclosure employ one or more of the following: one or more Demographic Measurements, one or more Patient Assessments, one or more Vital Signs, one or more Hematology Laboratory Values, one or more Chemistry Laboratory Values, and/or one or more Sepsis-associated biomarker concentrations. In particular embodiments, methods and systems of the disclosure employ some or all of (1) one or more Demographic Measurements, (2) one or more Patient Assessments, (3) one or more Vital Signs, (4) one or more Hematology Laboratory Values, (5) one or more Chemistry Laboratory Values, and/or (6) one or more Sepsis-associated biomarker concentrations. An example of Demographic Measurements includes at least age. An example of Patient Assessment includes at least Glasgow Coma Scale (GCS). Examples of Vital Signs includes Systolic Blood Pressure, Diastolic Blood Pressure, Temperature, Respiratory Rate, Heart Rate, Blood Oxygen Saturation (SpO2), and/or Inspired Oxygen Fraction (FiO2). Examples of Hematology Laboratory Values includes White Blood Cell Count, Platelet Cell Count, Lymphocyte Count, and/or Neutrophil Count. Examples of Chemistry Laboratory Values includes Creatinine, Blood Urea Nitrogen, Potassium, Chloride, Total Carbon Dioxide (TCO2), Sodium, Albumin, and/or Bilirubin. Examples of Sepsis-associated biomarkers includes C-Reactive Protein, Procalcitonin, and/or Lactate. Other examples of input features are provided in Tables 8-13.

In particular embodiments, a combination of patient characteristics are measured, assayed, analyzed, obtained, and/or considered to assess the presence or risk of having sepsis, or a certain stage of sepsis. In some embodiments, a combination of patient characteristics are measured, assayed, analyzed, obtained, and/or considered to determine the risk of having sepsis occurring in the next 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, or more hours. In some cases, a combination of patient characteristics are measured, assayed, analyzed, obtained, and/or considered to determine the risk of having sepsis occurring in the next 1-48, 1-36, 1-24, 1-12, 1-6, 1-2, 2-48, 2-36, 2-24, 2-12, 2-6, 6-48, 6-36, 6-24, 6-12, 12-48, 12-36, 12-24, 24-48, 24-36, or 36-48 hours.

The patient characteristics as input features may or may not be measured, assayed, analyzed, obtained, and/or considered in a particular order, such as a particular pre-ordained order, in certain aspects. The measuring, assaying, analyzing, obtaining, and/or considering of characteristic(s) for one patient may be different than the measuring, assaying, analyzing, obtaining, and/or considering of characteristic(s) for another patient, in specific cases. In some embodiments, one or more characteristics are measured, assayed, analyzed, obtained, and/or considered prior to one or more other characteristics. In certain embodiments, the nature of one or more characteristics triggers the consideration or need for consideration of one or more other characteristics, and such a progression may occur in real time. In some embodiments, the determination whether or not an individual has sepsis, has a certain stage of sepsis, or is at risk for sepsis (including within a certain time frame as noted above) may or may not be made only after measuring, assaying, analyzing, obtaining, and/or considering of at least, exactly, or no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or 58 or more patient characteristics.

In specific embodiments, one or more of the following patient characteristics are measured, assayed, analyzed, obtained, and/or considered to determine if a patient has sepsis, is at risk of developing sepsis, or has a particular sepsis stage: Age, albumin, bilirubin Total, blood urea nitrogen, C-reactive protein, chloride, creatinine, diastolic BP, Heart rate, lactate, lymphocyte, neutrophil, platelets, potassium, procalcitonin, respiratory rate, sodium, SpO2, Systolic BP, temperature, total carbon dioxide, and white blood cell count. In specific embodiments, one or more of the following patient characteristics are measured, assayed, analyzed, obtained, and/or considered to determine if a patient has sepsis, is at risk of developing sepsis, or has a particular sepsis stage: Age, albumin, angiopoietin-1, angiopoietin-2, Bilirubin Total, Blood Urea Nitrogen, C-Reactive Protein, CCL2/MCP-1, CCL20/MIP-3alpha, CCL4/MIP-1 beta, chloride, creatinine, CX3CL1/Fractalkine, CXCL10/IP-10, Diastolic BP, E-Selectin, FactorIII/Tissue Factor, Flt-3 Ligand, G-CSF, GM-CSF, Granzyme B, Heart Rate, IFN-alpha, IFN-gamma, IL-10, IL-15, IL-1beta/IL-1F2, IL-1ra/IL-1F3, IL-2, IL-4, IL-6, IL-7, IL-B/CXCL8, Lactate, Leptin, Lipocalin-2/NGAL, lymphocyte, neutrophil, PD-L1/B7-H1, Pentraxin-3, Platelets, Potassium, Procalcitonin, Respiratory Rate, Sodium, SpO2, Systolic BP, Temperature, TGF-alpha, thrombomodulin, TNF-alpha, Total Carbon Dioxide, TRAIL, TREM-1, VCAM-1, VEGF, White Blood cell count. A variety of patient characteristics that may be utilized are provided elsewhere herein, such as in any one of Tables 8-13.

In particular embodiments, the patient characteristics are input feature values for machine learning algorithms for sepsis risk diagnosis, sepsis stage determination, or sepsis risk prediction. In at least some embodiments, upon receiving a request (e.g., from a clinician's input device) to trigger a machine learning model to start the process of generating a determination or prediction, a provided system or systems initiate monitoring for input feature values from one or a variety of measurement devices or user input devices. Stated differently, in such embodiments, the provided systems may not continually monitor for input feature values such that input feature values are received at all times, but rather may monitor for input feature values once a request is received and until either the machine learning model generates a prediction or it is determined that the machine learning model is unable to generate a prediction based on the available input feature values. In other embodiments, the provided systems may continually monitor for input feature values. An input feature value collected by the measurement devices or user input devices is received at a provided system(s) (e.g., computing device) (e.g., near instantaneously) as the input feature value is collected (e.g., measured) such that the provided system(s) receive input feature values in real-time. As the input feature values are collected, a trigger logic determines if and when all of the input features to the machine learning model have an available value such that a prediction can be generated, which may include imputing a value to an input feature.

### III. Algorithm Description and Overview of Exemplary Workflow

In particular embodiments, the Sepsis ImmunoScore Algorithm is a process of using a set of live-stream measurements to generate a Sepsis ImmunoScore and its auxiliary components. In specific embodiments, inputs to the algorithm, including for example the core inputs, to the algorithm are live-stream measurements stored in a database. The core outputs of the algorithm may be the following:
1. Sepsis ImmunoScore sepsis risk score
2. Sepsis ImmunoScore risk stratification category
3. Input Features
   a. Value
   b. Imputed (True/False)
   c. SHAP value

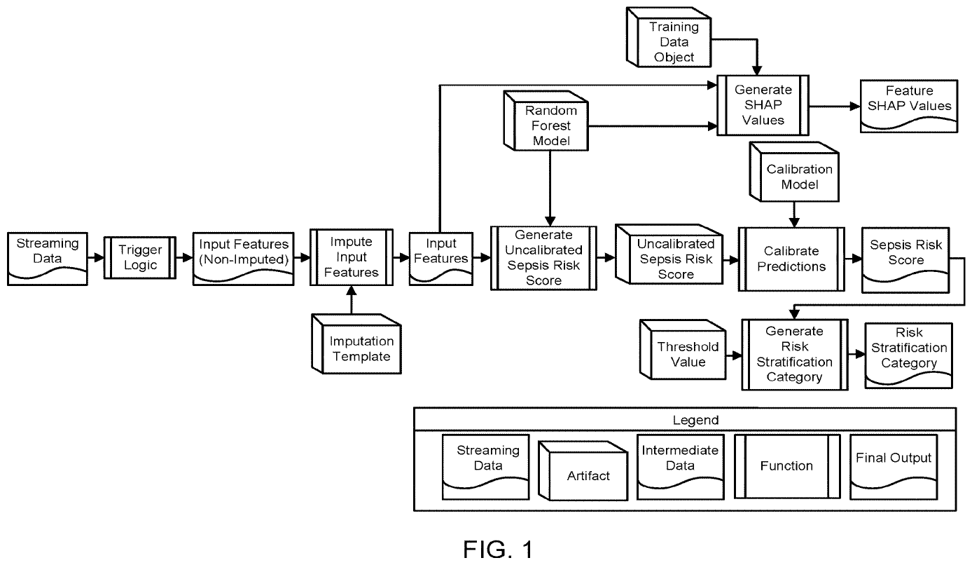

In particular embodiments, the algorithm comprises multiple functional components and artifacts that work together to generate an output, which may be considered the Sepsis ImmunoScore, and its auxiliary components. These functional components and artifacts reside inside the Backend and R engine modules of the SaMD specified in U.S. Provisional Patent Application No. 63/459,521, filed Apr. 14, 2023, which is incorporated by reference herein in its entirety. A visual depiction of one example of a Sepsis ImmunoScore Algorithm process can be found in FIG. 1.

A brief description of some functional components or artifacts for at least one example of an algorithm as depicted in FIG. 1 can be found in Table 1 and described following the table.

TABLE 1
Example of Overview of Sepsis ImmunoScore Algorithm Components -
An overview of the Sepsis Immunoscore algorithm components

| Component | Component Type | Input | Output | Description |
|---|---|---|---|---|
| Trigger Logic | Function | Streaming | Input Features | A function that |
| | | Data features | (Non-Imputed) | identifies |
| | | | | whether the |
| | | | | Sepsis |
| | | | | ImmunoScore |
| | | | | can be |
| | | | | generated and |
| | | | | processes the |
| | | | | streaming data |
| Impute Input | Function | Input Features | Input Features | The function |
| Features | | (Non-Imputed) | | that generates a |
| | | Imputation | | value for input |
| | | Template | | features missing |
| | | | | a value using a |
| | | | | predefined |
| | | | | imputation |
| | | | | template |
| Generate Un- | Function | Input Features | Uncalibrated | The function |
| calibrated | | Random | Sepsis | that will |
| Prediction | | Forest Model | Risk Score | generate an un- |
| | | | | calibrated |
| | | | | prediction using |
| | | | | supplied input |
| | | | | features and a |
| | | | | pretrained |
| | | | | random forest |
| | | | | machine |
| | | | | learning model |
| Generate | Function | Training Data | Feature SHAP | The function |
| SHAP Values | | Object | Value | that will |
| | | Input Features | | generate the |
| | | Random | | SHAP value |
| | | Forest Model | | associated with |
| | | | | each input |
| | | | | feature |
| Generate | Function | Sepsis | Risk | The function |
| Risk | | Risk Score | Stratification | that will |
| Category | | Threshold | Category | identify the risk |
| | | Value | | category for a |
| | | | | provided Sepsis |
| | | | | ImmunoScore |
| Calibrate | Function | Uncalibrated | Sepsis | The function |
| Predictions | | Sepsis | Risk Score | that will |
| | | Risk Score | | calibrate the |
| | | Calibration | | uncalibrated |
| | | model | | sepsis risk score |
| Imputation | Artifact | | | A pre-trained |
| Template | | | | template which |
| | | | | is used to |
| | | | | impute a value |
| | | | | for a missing |
| | | | | input feature |
| Random | Artifact | | | A pre-trained |
| Forest Model | | | | random forest |
| | | | | model which is |
| | | | | used to predict |
| | | | | the uncalibrated |
| | | | | probability of |
| | | | | sepsis within 24 |
| | | | | hours when |
| | | | | provided input |
| | | | | features |
| Calibration | Artifact | | | A pre-trained |
| Model | | | | logistic model |
| | | | | that will |
| | | | | perform Platt |
| | | | | calibration on a |
| | | | | given |
| | | | | uncalibrated |
| | | | | sepsis risk score |
| Threshold | Artifact | | | A list of three |
| Value | | | | values that |
| | | | | distinguish the |
| | | | | boundaries of |
| | | | | the Sepsis |
| | | | | ImmunoScore |
| | | | | risk |
| | | | | stratification |
| | | | | categories |
| Training Data | Artifact | | | The object |
| Object | | | | containing the |
| | | | | training data |
| | | | | input features |
| | | | | used to train the |
| | | | | pre-trained |
| | | | | Random Forest |
| | | | | Model |

i. Streaming Data and Input Features

In particular embodiments, the Sepsis ImmunoScore Algorithm receives and uses a variety of different streaming data features that include one or more of demographic measurements, patient assessments, vital signs, hematology laboratory values, chemistry laboratory values, and sepsis-associated biomarker concentrations. These input features may stem from clinical observations and/or FDA-approved devices, in certain examples.

In specific embodiments, the Sepsis ImmunoScore uses LOINC codes to identify the streaming data features. For each feature, a base LOINC code may be provided in the software. Because of the dynamic nature of the LOINC database, in differences in implementation of input features, the association between an input feature and the LOINC codes may be updated after release during installation and deployment, in certain embodiments.

The streaming data features may be converted to input features using the Trigger Logic function of the algorithm, in specific embodiments. Each input feature may have an associated Input Feature Selection methodology, which dictates how to select the input feature from a set of streamed measurements. In some embodiments, the Input Feature Selection methodology is intended to be fixed after the release of the Sepsis ImmunoScore SaMD. A detailed description of the process is specified in U.S. Provisional Patent Application No. 63/459,521, filed Apr. 14, 2023, which is incorporated by reference herein in its entirety.

A description of examples of input features is listed in Table 2 and Tables 8-13.

TABLE 2
Examples of Input Features - A list of examples of input features used
in a Sepsis ImmunoScore Algorithm, along with the type of measurement.

| | | | | Input Feature |
|---|---|---|---|---|
| | | Base | | Selection |
| Streamed Feature | Type | LOINC | Units | Methodology |
| Age | Demographic | | Years | Latest |
| | | | | Measurement |
| Glasgow Coma Scale | Patient | 9269-2 | — | Max value in |
| | Assessment | | | prior 6 hour |
| | | | | period |
| Systolic Blood | Vital Sign | 8480-6 | mm Hg | Min value in |
| Pressure | | | | prior 6 hour |
| | | | | period |
| Diastolic Blood | Vital Sign | 8462-4 | mm Hg | Min value in |
| Pressure | | | | prior 6 hour |
| | | | | period |
| Body Temperature | Vital Sign | 8310-5 | Celsius | Max value in |
| | | | | prior 6 hour |
| | | | | period |
| Respiratory Rate | Vital Sign | 9279-1 | Breaths per | Max value in |
| | | | minute | prior 6 hour |
| | | | | period |
| Heart Rate | Vital Sign | 8867-4; | Beats per | Max value in |
| | | 8889-8 | minute | prior 6 hour |
| | | | | period |
| Blood oxygen | Vital Sign | 59408-5 | % | Min value in |
| saturation (SpO₂) | | | | prior 6 hour |
| | | | | period |
| White Blood Cell | Hematology | 26464-8 | 10⁹/L | Latest |
| Count | Laboratory | | | Measurement |
| | Measurement | | | |
| Lymphocyte Count | Hematology | 26474-7 | 10⁹/L | Latest |
| | Laboratory | | | Measurement |
| | Measurement | | | |
| Neutrophil Count | Hematology | 26499-4 | 10⁹/L | Latest |
| | Laboratory | | | Measurement |
| | Measurement | | | |
| Platelet Count | Hematology | 26515-7 | 10⁹/L | Latest |
| | Laboratory | | | Measurement |
| | Measurement | | | |
| Creatinine | Chemistry Panel | 38483-4 | mg/dL | Latest |
| | Measurement | | | Measurement |
| Blood Urea Nitrogen | Chemistry Panel | 6299-2 | mg/dL | Latest |
| | Measurement | | | Measurement |
| Potassium | Chemistry Panel | 77142-8 | mmol/L | Latest |
| | Measurement | | | Measurement |
| Chloride | Chemistry Panel | 77138-6 | mmol/L | Latest |
| | Measurement | | | Measurement |
| Total Carbon Dioxide | Chemistry Panel | 2028-9 | mmol/L | Latest |
| | Measurement | | | Measurement |
| Sodium | Chemistry Panel | 77139-4 | mmol/L | Latest |
| | Measurement | | | Measurement |
| Albumin | Chemistry Panel | 1751-7 | mg/dL | Latest |
| | Measurement | | | Measurement |
| Bilirubin Total | Chemistry Panel | 1975-2 | mg/dL | Latest |
| | Measurement | | | Measurement |
| Procalcitonin | Sepsis- | 33959-8 | pg/mL | Latest |
| | Associated | | | Measurement |
| | Biomarker | | | |
| C-Reactive Protein | Sepsis- | 1988-5 | pg/mL | Latest |
| | Associated | | | Measurement |
| | Biomarker | | | |
| Lactate | Sepsis- | 33959-8; | mmol/L | Latest |
| | Associated | 2524-7 | | Measurement |
| | Biomarker | | | |
| Inspired Oxygen | Vital Sign | 3150-0 | % | Max value in |
| Percentage (FiO₂) | | | | prior 6 hour |
| | | | | period |

In particular embodiments, an output is used to place the individual within one of four risk stratification categories: Low, Medium, High, or Very High. The risk stratification categories may be associated with the prevalence of the following adverse clinical events with respect to the order time of the Sepsis ImmunoScore:
1. In-hospital mortality
2. Readmission within 30 days
3. Transfer of care to the ICU within 24 hours
4. Placement on mechanical ventilation within 24 hours
5. Usage of vasopressors within 24 hours (e.g., norepinephrine, vasopressin, Epinephrine, dopamine, angiotensin II, terlipressin, Selepressin)

The stratification bands may be bounded by three thresholds identified during the development of the Sepsis ImmunoScore Algorithm. The calculation for identifying the risk stratification category is described as follows:

A receiver operating characteristic (ROC) curve was generated using the out-of-bag uncalibrated predictions against the training label during the development of the Sepsis ImmunoScore Algorithm. This ROC curve was used to identify three thresholds with the following criteria:
1. Lowest Threshold—Identifying the threshold with the highest sensitivity whilst maintaining a false positive rate of 50%.
2. Optimal Threshold—Threshold is identified as the ‘top-left’ point of the ROC curve, i.e., the point on the ROC curve with minimal Euclidean distance to (0,1).
3. 95th percentile threshold—Threshold corresponding to the 95th percentile of sepsis risk scores within the training dataset.

In specific embodiments, possible values of outputs are provided in Table 3.

TABLE 3
Examples of Values of Outputs

| Output | Possible | User Interpretation |
|---|---|---|
| Sepsis Risk Score | 0-100 | Probability of having or |
| | | developing sepsis within 24 |
| | | hours of the Sepsis |
| | | ImmunoScore being ordered. |
| Risk Stratification | Low | Each risk stratification |
| Category | Medium | category has associated |
| | High | diagnostic performance and |
| | Very high | associated average |
| | | prognostic metrics (see |
| | | immediately below) |

Examples of a diagnostic interpretation and recommended action of each of the potential risk stratification categories as an output is provided in Table 4.

TABLE 4
Risk Stratification Categories and Examples of Associated Scores

| Risk Stratification | Diagnostic | Sepsis Risk Score |
|---|---|---|
| Category | Interpretation | Range |
| Low | Sepsis unlikely | 0%-12.2% |
| Medium | Sepsis possible | 12.2%-30.6% |
| High | Sepsis likely | 30.6%-87.2% |
| Very High | Sepsis very likely | 87.2%-100% |

A sepsis risk score exactly equal to the threshold (e.g., 12.2%) is placed in the higher risk stratification category.

Various embodiments provide assessment of the presence or risk of sepsis in an individual based on one or a variety of factors encompassed in any one of Tables 8-13 (which may be referred to as input features given their diversity in nature (e.g., demographic, patient assessment, vital sign, hematological laboratory measurement, chemistry panel measurement, sepsis-associated biomarker, etc.), one or a variety of factors encompassed in any one of Tables 8-13 (which may be referred to as biomarkers, given their proteinaceous nature), or both. In some embodiments, one or more factors in any one of Tables 8-13 are utilized to generate an output that determines the presence or risk for sepsis.

### III.A. Examples of Input Features

In particular embodiments, one or more of the following input features are utilized in assessing whether or not an individual has sepsis or is at risk for having sepsis, such as within 24 hours of measurement.

i. Demographic Measurements

Age can be calculated as the [current date−date of birth]. Age has been traditionally considered a risk factor, as the incidence of sepsis increases as patients age. This may be associated with an increase in chronic conditions that are important in the progression or presence of sepsis, in specific embodiments.

ii. Patient Assessments

Glasgow Coma Scale (GCS) is an assessment for cognitive impairment performed by a certified individual. GCS is recorded on a scale of 3-15 and is assessed by a health care professional. GCS is intended to assess the Central Nervous System component of the Sequential Organ Failure Assessment (SOFA) Score (Jones et al., Crit Care Med. 2009 May; 37(5): 1649-1654, which is incorporated by reference herein in its entirety). The SOFA score allows one to monitor a person's status to determine the extent of a person's organ function or rate of failure. The score is based on six different scores, one score for each of the respiratory, cardiovascular, hepatic, coagulation, renal, and neurological systems. In specific embodiments, sepsis is when the individual has Organ Dysfunction (Acute Change in SOFA Score ≥2), and septic shock is when the individual has Organ Dysfunction (Acute Change in SOFA Score ≥2) and Refractory Hypotension.

iii. Vital Signs

### 1. Systolic Blood Pressure

Systolic Blood Pressure is the pressure exerted when the heart beats and blood is ejected into the arteries. It can be measured using a machine like a sphygmomanometer. Systolic Blood Pressure is used in the calculation of the Mean Arterial Pressure. Mean Arterial Pressure is intended to assess the Cardiovascular system component of the SOFA score.

### 2. Diastolic Blood Pressure

Diastolic Blood Pressure is the pressure within the arteries when the heart rests between beats. It can be measured using a machine like a sphygmomanometer. Diastolic Blood Pressure is used to calculate the Mean Arterial Pressure. Mean Arterial Pressure is intended to assess the Cardiovascular system component of the SOFA score.

### 3. Temperature

Temperature is the specific degree of hotness in the body. Temperature can be measured in various ways; one common method is a thermometer. Temperature can be used to identify if a patient has a fever. Fever patterns can be used to identify possible causes of infections, as remittent fevers (fevers that fluctuate more than 1.1° C. and never return to normal) are often caused by infections.

### 4. Respiratory Rate

Respiratory rate is defined as the number of beats per minute. Respiratory rate can be measured manually, or through a machine such as a pulse oximeter. An abnormal respiratory rate can be an indication a patient contains an irregular amount of oxygen available. Tachypnea (elevated respiratory rate) is commonly observed within septic patients and is associated with an increase in 28-day mortality in septic patients. Respiratory rate is also associated with SIRS, which is part of the older Sepsis-2 definition.

### 5. Heart Rate

Heart rate is measured as the number of heartbeats within a certain period. Heart rate can be measured manually, or through a machine such as a heart rate monitor. An irregular heart rate may be associated with poor blood circulation and either hypotension or hypertension. Heart Rate is also associated with SIRS, which is part of the older Sepsis-2 definition.

### 6. Blood Oxygen Saturation (SpO2)

SpO2 is the measure of the amount of oxygen-carrying hemoglobin in the blood relative to non-oxygen-carrying hemoglobin. SpO2 can be calculated and measured in many methods, one common way being the use of a pulse oximeter. SpO2 is a non-invasive method to estimate the arterial oxygen saturation (SaO2), which is used in the calculation of PaO2/FiO2 of the respiratory system SOFA score calculation.

### 7. Inspired Oxygen Fraction (FiO2)

FiO2 is the concentration of oxygen in the inspired gas mixture. The gas mixture of room air has a fraction of inspired oxygen of 21%. An elevation of FiO2 may indicate a patient requires a higher percentage of oxygen. FiO2 is used in the calculation of PaO2/FiO2 of the respiratory system SOFA score calculation.

iv. Hematology Laboratory Values

### 1. White Blood Cell Count

White blood cell count is a measure of the number of white blood cells in a patient's blood. White blood cell count can be measured using a hematology laboratory test. An irregularity in white blood cell count can indicate a person is fighting an infection.

### 2. Platelet Cell Count

Platelet Cell count is a measure of the number of platelet cells in a patient's blood. Platelet cell counts can be measured using a hematology lab test. An irregularity in Platelet cell counts can indicate if an individual has difficulty with clotting. Platelets are involved in both hemostasis and the immune response and play a critical role in sepsis. Platelets are used to assess the coagulation system component of the SOFA Score.

### 3. Lymphocyte Count

Lymphocyte count is a measure of the number of lymphocyte cells in a patient's blood. Lymphocyte cell counts can be measured using a hematology lab test. Lymphocyte cells are a type of white blood cell that is a part of the immune system. An irregularity in the lymphocyte count can indicate the presence of an infection. Lymphocytes combined with neutrophils can be used to calculate the Neutrophil to Lymphocyte Ratio (NLR). NLR is a metric that is associated with mortality in septic patients, as non-survivors exhibit a significantly higher NLR.

### 4. Neutrophil Count

The neutrophil count is a measure of the number of neutrophil cells in a patient's blood. Neutrophil cell counts can be measured using a hematology lab test. Neutrophils are a type of granulocyte, which is a type of white blood cell that is a part of the immune system. An irregularity in the lymphocyte count can indicate the presence of an infection. Lymphocytes combined with neutrophils can be used to calculate the Neutrophil to Lymphocyte Ratio (NLR). NLR is a metric that is associated with mortality in septic patients, as non-survivors exhibit a significantly higher NLR.

v. Chemistry Laboratory Values

### 1. Creatinine

Creatinine is defined as the concentration of creatinine within the blood. Creatinine measurements in the blood can be performed using a chemistry laboratory panel. High creatinine in the blood indicates the renal system may not be filtering creatinine into the urine for elimination. Creatinine is used to assess the renal system component of the SOFA Score.

### 2. Blood Urea Nitrogen

Blood Urea Nitrogen (BUN) is the amount of urea nitrogen that remains in the blood. BUN measurements in the blood can be performed using a chemistry laboratory panel. Urea Nitrogen is a waste compound that is transferred from the blood into the urine by the kidneys. A high BUN may indicate the renal system may not be operating effectively. An elevated BUN has been linked to the development or presence of sepsis.

### 3. Potassium

Potassium is measured as the concentration of potassium ions within the blood. Potassium measurements in the blood can be obtained by performing a chemistry laboratory test. Potassium plays a role in maintaining fluid levels within the body's cells. An imbalance in potassium can result in an imbalance in fluid levels. Disturbances in fluid levels have been associated with many patients admitted into the ICU, and fluid resuscitation is effective for treating patients with severe sepsis or septic shock.

### 4. Chloride

Chloride is measured as the concentration of chloride ions present within the blood. Chloride measurements in the blood can be obtained by performing a chemistry laboratory test. Chloride plays a role in maintaining fluid levels within the body's cells. An imbalance in chloride can result in an imbalance in fluid levels. Disturbances in fluid levels have been associated with many patients admitted into the ICU, and fluid resuscitation is effective for treating patients with severe sepsis or septic shock.

### 5. Total Carbon Dioxide (TCO2)

Total Carbon Dioxide is the measure of carbon dioxide found in the blood. TCO2 can be measured in the blood using a chemistry laboratory test. TCO2 can be used to identify metabolic acidosis which is a function of the respiratory system. An imbalance in TCO2 can be linked with an irregularity in the respiratory system. Elevated TCO2 measurements have been linked to an increase in mortality for sepsis patients.

### 6. Sodium

Sodium is measured as the concentration of chloride ions present in the blood. Sodium measurements in the blood can be obtained by performing a chemistry laboratory test. Sodium plays a role in maintaining fluid levels within the body's cells. An imbalance in potassium can result in an imbalance in fluid levels. Disturbances in fluid levels have been associated with many patients admitted into the ICU, and fluid resuscitation can be effective for treating patients with severe sepsis or septic shock.

### 7. Albumin

Albumin is measured as the concentration of albumin present in the blood. Albumin measurements in the blood can be obtained by performing a chemistry laboratory test. Albumin is a protein that is synthesized by the liver that minimizes the capillary leak into the body's tissues. An irregularity in Albumin can indicate a problem with the liver or renal system. Serum albumin has been identified as a predictor of mortality for septic patients.

### 8. Bilirubin

Bilirubin is measured as the concentration of bilirubin present in the blood. Bilirubin measurements in the blood can be obtained by performing a chemistry laboratory test. Bilirubin is a protein that is generated during the breakdown of red blood cells. Bilirubin is a waste product that is filtered out by the liver in healthy patients. Bilirubin is used in the assessment of the liver component in the SOFA Score calculation.

vi. Sepsis-associated Biomarker Concentrations

### 1. C-Reactive Protein

C-reactive Protein (CRP) is the concentration of CRP within the blood. CRP measurements in the blood can be obtained by performing a chemistry laboratory test. CRP is a protein that is produced by the liver whose level rises due to inflammation. Studies have shown that CRP when combined with temperature, can increase the detection of infection.

### 2. Procalcitonin

Procalcitonin (PCT) is measured as the concentration of PCT present in the blood. PCT can be measured by a laboratory test. PCT is released by tissues into the bloodstream during the presence of inflammation. Bacterial infections have shown to have a large impact on PCT synthesis, as bacterial endotoxins and cytokines facilitate host cells' PCT secretion.

### 3. Lactate

Lactate is measured as the concentration of lactate present in the blood. Lactate can be measured by a laboratory test. Lactate (or lactic acid) is generated by tissues during states of hypoxia. The build of lactate within the blood can indicate a patient is under organ failure or shock. Lactate has emerged as a part of the septic shock label in the Sepsis-3 definition.

In some embodiments, an individual at risk for sepsis or suspected of having sepsis has analyzed, measured, considered, and/or assayed one or more samples. Examples of samples include blood, plasma, serum, urine, tissue, skin, saliva, phlegm, and/or mucus. The level of any one or more patient characteristics from the sample(s) may be utilized input features in a Model encompassed herein. Alternatively, or in addition, an individual may be subjected to blood pressure tests, imaging studies (X-ray, ultrasound scan or computerized tomography (CT) scan), and so forth.

In some embodiments, a plurality of input features are selected from 1, 2, 3, or more categories, and such features may or may not be used with any model encompassed herein. In specific embodiments, a plurality of input features includes at least one input feature of a first group, at least one input feature of a second group, and at least one input feature of a third group. In some embodiments, the first group comprises the input features in the group consisting of age, sex at birth, race, ethnicity, past medical history of the patient, current complaints or symptoms of the patient, neurological assessments, clinical decision support alerts, clinician or other chart notes, diagnosis codes, procedures performed on the patient, current medications of the patient, interventions, patient care setting, medical imaging data or assessments, electrograms, endoscopic tests, systolic blood pressure, diastolic blood pressure, body temperature, respiratory rate, heart rate, blood oxygen saturation, a fraction of inspired oxygen, and a combination thereof. In some embodiments, the second group comprises the input features in the group consisting of white blood cell count, lymphocyte count, neutrophil count, platelet count, creatinine, blood urea nitrogen, potassium, chloride, total carbon dioxide, sodium, albumin, bilirubin, lactate, glucose, calcium, hematocrit, hemoglobin concentration, lab-derived biomarkers, clinical severity measures and composites, biopsies data, microorganism test data, and a combination thereof. In some embodiments, the third group comprises the input features in the group consisting of procalcitonin, C-reactive protein, protein biomarkers, genomic biomarkers, gene expression or transcriptomic biomarkers, composite biomarker scores, and a combination thereof.

In specific embodiments, there are the following categories, and one or more input feature from any one or more of the categories may be utilized in determining whether an individual has sepsis or is at risk of having sepsis within a pre-determined time:

### Category 1

vitals (e.g., temperature, heart rate, blood pressure, respiratory rate, oxygen saturation)
demographics (e.g., age, sex, race, ethnicity)
past medical history
current complains or symptoms
neurological assessments (Glasgow Coma Scale, Full Outline of Unresponsiveness (FOUR)
clinical decision support alerts
clinician or other chart notes
diagnosis codes (e.g., ICD-10)
procedures (including CPT codes)
current medications (including NDC codes)
interventions
patient care setting (ICU, ED, hospital floor, outpatient clinic, etc.)
imaging (e.g., X-ray, CT scan, MRI, fMRI, ultrasound)
electrograms (e.g., EEG, EKG)
endoscopic tests

### Category 2

labs
lab-derived biomarkers (e.g., monocyte distribution width, Cytovale IntelliSep) clinical severity measures and composites (e.g., Sequential Organ Failure Assessment (SOFA) score, SOFA component scores, Charlson Comorbidity Index, Acute Physiology and Chronic Health Evaluation (APACHE) I and II),
biopsies
microorganism tests (cultures, PCR tests, antigen tests)

### Category 3

protein biomarkers (including Angiopoietin-1, Angiopoietin-2, C-Reactive Protein, Cystatin C, D-Dimer, E-Selectin, Fractalkine, FLT3-Ligand, GCSF, GDF15, GMCSF, Granzyme-B, IFN-alpha, IFN-gamma, IL1-beta, IL1-RA, IL-2, IL-4, IL-6, IL-7, IL-8, IL-10, IL-15, IP-10, lactate dehydrogenase (LDH), lipopolysaccharide-binding protein (LBP), Leptin, MCP1, MIP1-alpha, MIP1-beta, MIP3-alpha, NGAL, Pancreatic Stone Protein, PDL-t, Pentraxin-3, Procalcitonin, Protein C, S100b, TGF-alpha, Thromodulin, Tissue Factor, TNF-alpha, TRAIL, TREM-1, Troponin, VCAM-1, VEG-F)
genomic biomarkers (e.g., one or more SNPs in CD14, TLR1, TLR2, TLR4, TLR6, TNF-alpha, IL-6, factor V, factor XII, FER, MAN2A1)
gene expression or transcriptomic biomarkers (including CEACAM4, LAMPt, PLAC8, PLA2G7, CEACAMI, ZDHHC19, C9orf95, GNA15, BATF, C3AR1, KIAA1370, TGFBI, MTCH1, RPGRIP1, HLA-DPB1, Nuclear factor-kappa B (NF-kB), Heme oxygenase-1 (HO-1), FCGR2C, CRP, IL-6, IL-10, IL-12, TNF, LY6E, LAMP3, ISG15, USP18, RSAD2, IFI44L, NCOA7, CMPK2, IFI44, BATF2, GBP4, IFIH1, GMPR, CXCL10, IFI27, ISG15, CCL2, OAS3, P2RY14, GCH1, CD274, IDO1, ARL4A, BPGM, TNFAIP3, TNFAIP6, ILIR1, PROK2, RGS1, CXCR6, RTP4, TLR3, FZD5, KCNJ2, IRAK2, RGS16, CXCL8, OLR1, CCRL2, SPP1, ST3GAL4, CST7, IL1R2, IL4R, GALM, CTLA4, SOCS1, BCL2L1, TGM2, SLC39A8, CA2, PNP, GATA1, SLC1A5, RGS16, CD44, PDGFC, CSF2RA, CXCL1, CSF3R, SOCS3, IL18R1, PIM1, LEPR, IL1B, CSF2RB, ITGB3, STAT1, FAS, EFNA1, IER3, EETS2, SLC2A3, BCL2A1, PFKFB3, CEBPB, BIRC2, ATF3, TUBB2A, GOS2, MERTK, BIK, QSOX1, PYGL, TGFA, P4HA2, LDHA, IRS2, TSPO, HGF, GADD45A, TGFBR3, CD38, PRF1, FASLG, TIMP3, ANKH, LGALS3, PPP2R5B, GPX1, BNIP3L, BCL2L1, KLF7, FOSL2, ADM, MXI1, SELENBP1, PGF, FOXO3, NEDD4L, SLC2A1, SIAH2, MMP9, CXCR3, CD8A, IFNG, CD8B, JAK2, CCL4, ICAM1, WARS1, KRT1, GPR65, DYRK3, ACHE, ARG1, LCN2, LTF, OLFM4, HLA-DMB, YKT6, PDE4B, TWISTNB, BTN2A2, ZBTB33, PSMB9, CAMK4, TMEM19, SLC12A7, TP53BP1, PLEKHO1, SLC25A22, FRS2, CD24, STX1A, KCNMB4, CRISP2, HTRA1, PPL, RHBDF2, ZCCHC4, DDX6, SENP5, RAPGEF1, DTX2, RELB, DYRK2, CCNB1IP1, TDRD9, ZAP70, ARL14EP, MDC1, ADGRE3
composite biomarker scores (e.g., SeptiCyte LAB and Septicyte RAPID, Inflammatix TruVerity)

### III.B. Algorithm Output

In particular embodiments, core outputs of the Sepsis ImmunoScore Algorithm are as follows:
1. Sepsis ImmunoScore sepsis risk score
2. Sepsis ImmunoScore risk stratification category
3. Input Features
   a. Value
   b. Imputed (True/False)
   c. SHAP value
      i. Sepsis ImmunoScore

In particular embodiments, the Sepsis ImmunoScore sepsis risk score is a continuous value between 0 and 100 and is the output of the Calibrate Predictions function specified elsewhere herein. The Sepsis ImmunoScore, in specific embodiments, is intended to be displayed to users of the SaMD. The Sepsis ImmunoScore may be interpreted as the probability of a patient having or developing sepsis, as defined by Sepsis-3, within 24 hours from the order time of the Sepsis ImmunoScore, in certain embodiments. In specific embodiments, the Sepsis ImmunoScore may differ from the uncalibrated sepsis risk score by imposing a calibration that refines the interpretability of the uncalibrated sepsis risk score.

ii. Risk Category

In particular embodiments, the Sepsis ImmunoScore is used to place the patient within one of four risk stratification categories: Low, Medium, High, or Very High. The risk stratification categories are associated with the prevalence of the following adverse clinical events with respect to the order time of the Sepsis ImmunoScore:
1. In-hospital mortality
2. Readmission within 30 days
3. Transfer of care to the ICU within 24 hours
4. Placement on mechanical ventilation within 24 hours
5. Usage of vasopressors within 24 hours

In certain embodiments, the risk stratification categories are delineated by three thresholds identified during the development of the Sepsis ImmunoScore Algorithm. The calculation for identifying the risk stratification category is described elsewhere herein.

iii. Input Features

In particular embodiments, the Sepsis ImmunoScore will retain the input features' value used to calculate the Sepsis ImmunoScore, a TRUE or FALSE value indicating if the value was imputed, and the associated SHAP value. A SHAP value is a numerical value that signifies the local feature importance of the Sepsis ImmunoScore for a particular set of input features. In certain aspects, the use of the SHAP values aid in the interpretation and trust of the Sepsis ImmunoScore. In particular embodiments, the SHAP values for a single Sepsis ImmunoScore when added equal to the uncalibrated Sepsis ImmunoScore prediction−the mean (pre-calibrated Sepsis ImmunoScore prediction) across the predefined dataset. From here, the additive impact of each input feature on the pre-calibrated Sepsis ImmunoScore prediction can be observed.

The SHAP values are not a global algorithm property, but a property of any new generated score, and localize the commonly used concept of feature importance for an individual score, in some embodiments.

### III.C. Functional Components

iv. Trigger Logic

### Inputs

Streaming Data Features

### Outputs

Identify if the Sepsis ImmunoScore can generate a result
Input Features with missing values
   The trigger logic can identify if an active Sepsis ImmunoScore order is ready to generate the Sepsis ImmunoScore. Once the trigger logic indicates the Sepsis ImmunoScore can be generated, the trigger logic will pass the input features to the Imputation functional component, in certain embodiments. More information on the specification and description of the trigger logic can be found in U.S. Provisional Patent Application No. 63/459,521, filed Apr. 14, 2023, which is incorporated by reference herein in its entirety.
   v. Impute Input Features

### Inputs

Input Features with Missing Values

### Outputs

Input Features with no Missing Values

### Artifacts

### Imputation Template

The Impute Input Features function takes the input features identified from the Trigger Logic Module to impute any missing values. The Impute Input Features may perform the following steps in order, in specific embodiments:
1. Impute missing FiO2 measurements as 21
2. Impute missing GCS measurements as 15
3. Perform bag imputation using the imputation template.
   Further information on the methodology of imputation can be found elsewhere herein.
   vi. Generation of Uncalibrated Prediction

### Inputs

Input features, with missing values imputed.

### Outputs

Uncalibrated Sepsis ImmunoScore. Raw model prediction score that has not been calibrated

### Artifacts

Machine Learning Model

In particular embodiments, the Generate Pre-Calibrated Prediction function uses the output from the Impute Input Features function along with a trained machine learning model to generate the raw pre-calibrated Sepsis ImmunoScore as a continuous value between [0,1]. The machine learning model used may be a random forest model, and is implemented using the Ranger R Package. More information about the machine learning model can be found elsewhere herein.

vii. Calibrate Predictions

### Inputs

Uncalibrated sepsis risk score

### Outputs

Sepsis risk score

### Artifacts

Calibration Model

The Calibrate Predictions function uses the uncalibrated Sepsis ImmunoScore along with a calibration model to generate the Sepsis ImmunoScore. More information about the calibration model can be found elsewhere herein.

viii. Generate SHAP Values

### Inputs

Input Data (Imputed)

### Outputs

SHAP Values

### Artifacts

Machine Learning Model
Training Data Object

In certain embodiments, the Generate SHAP Values function uses the imputed input features, the trained machine learning model, and the training object to generate the SHAP value contributions of the input features. In specific aspects, calculations of SHAP values use a game-theoretic approach to identify the contribution a feature plays in the prediction of an observation (Scott, Lundberg M, and Lee Su-In. “A Unified Approach to Interpreting Model Predictions.” 31st Conference on Neural Information Processing Systems, 2017). The SHAP Values may use the training data object to identify the estimations of the feature contribution to the training dataset, in specific embodiments. Due to the computational complexity of calculating SHAP values, the Generate SHAP Values function may use a SHAP estimation implementation via the fastshap R package, in at least some cases. In some embodiments, the fastshap R package will use Monte-Carlo simulations with 10 rounds to estimate the feature contribution of the training data object with a fixed seed. This estimation can be applied to new observations to generate the overall contribution to a prediction, in some embodiments.

ix. Generate Risk Stratification Category

### Inputs

Sepsis risk score

### Outputs

Risk Stratification Category

### Artifacts

Threshold List

In certain embodiments, the Generate Risk Stratification Category function uses the pre-established threshold list to place the Sepsis ImmunoScore sepsis risk score value into one of four categories. The Generate Risk Stratification Category function will place a patient in a risk category with the following logic:

TABLE 5
Generate Risk Stratification Category

| Sepsis ImmunoScore | Category |
|---|---|
| 95^{th} Percentile Threshold <= Score | Very High |
| Optimal Threshold <= Score & Score < 95^{th} | High |
| Percentile | |
| Lowest Threshold <= Score & Score < | Medium |
| Optimal Threshold | |
| Score < Lowest Threshold | Low |

### III.D. Artifacts

The Sepsis ImmunoScore Algorithm comprises 5 artifacts, in certain embodiments:
1. An imputation template
2. A pre-trained machine learning algorithm
3. A calibration model
4. A training data object
5. A threshold list

All artifacts may be created during the development of the Sepsis ImmunoScore Algorithm. In particular embodiments, they are intended to be fixed upon release of the Sepsis ImmunoScore SaMD.

i. Imputation Template

The imputation template is a pre-trained template used to impute a value for a missing input feature, in specific embodiments. The imputation template may be stored as an R data structure file. In an embodiment, the purpose of this artifact is to ensure all input features have an available measurement before use in generating the uncalibrated sepsis risk score.

ii. Pre-Trained Machine Learning Model

In certain embodiments, the pre-trained machine learning model is a supervised machine learning model built using a training dataset containing input features and a training label in conjunction with hyperparameters. The machine learning model is stored as an R data structure file, in particular aspects. The purpose of the machine learning model is to learn the relationship between the input features and the risk of having or developing sepsis within 24 hours of the Sepsis ImmunoScore order time, in particular embodiments. This relationship can be applied to future, unobserved patients with corresponding input features, in at least some cases.

iii. Calibration Model

The calibration model is a logistic model that will use the uncalibrated sepsis risk score as an input to generate the Sepsis ImmunoScore sepsis risk score, in particular embodiments. The calibration model may be saved as an R data structure file. In one embodiment, the purpose of the calibration model is to establish a relationship between the uncalibrated sepsis risk score and the classification of having or developing sepsis within 24 hours of the Sepsis ImmunoScore order. This relationship can be applied to convert future uncalibrated sepsis risk score into the sepsis risk score, in certain embodiments.

iv. Training Data Object

In particular embodiments, the training data object is a data frame containing the input features of the training dataset. The training data object may be saved as an R data structure file. In some embodiments, the purpose of the training data object is to provide data to learn the relationship between an input feature's SHAP value with its corresponding uncalibrated sepsis risk score value. This relationship can be applied to future Sepsis Immunoscore orders to generate the SHAP values for a corresponding sepsis risk score, in some embodiments.

v. Threshold List

In some embodiments, the threshold list is a vector that contains three threshold values between 0 and 1 which are associated with the boundaries of the Sepsis ImmunoScore Risk Categories. The threshold list may be saved in a JSON file. In specific embodiments, the three values will be ranked in ascending order and are referred to as “Lowest”, “Optimal”, and “95th Percentile” threshold, respectively.

### III.E. Dataset for Development

The algorithm was developed using retrospective data from de-identified patient data and samples originating from the multicenter NOSIS Dataset and Biobank.

In particular embodiments, the NOSIS Dataset and Biobank is a unified dataset comprised of prospectively collected clinical data (Electronic Medical Records data), time-series biological samples, and biomarker measurements obtained from these samples. The dataset is built through prospective clinical studies sponsored by the Applicant at a consortium of clinical sites.

### III.F. Algorithm Artifacts Design

The methods used to design each of the Sepsis ImmunoScore Algorithm functions and artifacts are defined in the following sections.

i. Imputation Template

In some embodiments, the imputation template is generated by the use of bag imputation upon the Sepsis ImmunoScore Algorithm training dataset identified elsewhere herein. Bag imputation is a statistical method that builds a random forest model for each input feature in the Sepsis ImmunoScore Algorithm, in specific embodiments. Each random forest model may use the remaining observed input features to generate an imputed value. In some embodiments, the imputation template can be evaluated by performing on unseen data and calculating the R2 of the imputed measurement vs known measurements for each input feature.

The implementation of bag imputation was performed by using the imputeMissings R package.

The data used for development of the imputation template is the same as the one used for the machine learning model, in certain embodiments.

ii. Pre-Trained Machine Learning Model

The pre-trained machine learning model may be developed by training a supervised machine learning model using a training dataset containing input features and a training label in conjunction with the hyperparameters associated with the machine learning model. In certain embodiments, the pre-trained machine learning model is evaluated by looking at specific performance criteria associated with diagnostic and prognostic performance.

iii. Machine Learning Model

In specific embodiments, the machine learning model used is a random forest model. A random forest model is a popular ensemble model combining many simple tree models to generate a prediction prediction (Breiman, Leo. “Random forests.” Machine learning 45, 2001, pp. 5-32). Random forest performs bagging, a method of sampling a dataset with replacement. An individual simple model may be trained on this sampled dataset. In specific embodiments, this sampling with replacement followed by training is performed n times to generate an ensemble, or forest, of simple models. The random forest used for the development of the Sepsis ImmunoScore algorithm used 1000 regression tree models as the base model to generate a probability forest (Malley, James D., Jochen Kruppa, Abhijit Dasgupta, Karen G. Malley, and Andreas Ziegler. “Probability machines.” Methods of information in medicine 51, no. 01, 2012, pp. 74-81), in certain embodiments. The random forest model was trained by maximizing the area under the receiver operating characteristic curve (AUROC) between the predictions and training label, in specific embodiments.

A benefit of random forest models is the use of out-of-bag (OOB) error. Due to the bagging method of generating each decision tree, there is a probability for an observation to not be included in the training of a decision tree. This set of observations that are not included may be defined as OOB in certain embodiments. In specific embodiments, by generating a prediction for the OOB, one can identify the error for all observations instead of relying upon a traditional train/test split.

The implementation of random forest was performed by using the ranger R package.

iv. Training Dataset

The training dataset is a subset of data from the NOSIS Dataset and Biobank, and was extracted with the following criteria:

This was a multicenter prospective, observational study of patients with suspected sepsis. Patients were identified by querying the hospital electronic medical record system for study entry criteria. Patients who had a relevant order for a test indicating suspected bacteremia (blood culture) were eligible for inclusion. A list of eligible patients was created, and research or hospital personnel evaluated for the presence of remnant samples from a lithium heparin with gel separator (light green top) tube in the clinical lab. Patients with a sample that was available and collected were included as part of the NOSIS data set.

Adult patients (aged 18 or older) presenting to the emergency department or in inpatient settings who were suspected of sepsis were included. Suspicion of sepsis was inferred from the order of a blood culture. Patient data were extracted from their hospital electronic health records (EHRs), including demographic data, comorbidities, vitals, lab results, electronically documented interventions, and ICD-10 codes. In addition, remnant plasma samples leftover from blood drawn during routine clinical care each comprising four 175 μL aliquots were collected, frozen at −80° C., and shipped to a central laboratory. Each sample comprises at least one aliquot of plasma frozen at −80° C. from when samples were available up to 3 days prior to enrollment and prospectively during hospitalization.

One plasma discard sample per patient was assayed for approximately 40 protein biomarkers for a subsample of patients in the NOSIS biobank, including CRP and PCT. Operating sequentially for each site based on the date of patient enrollment, we selected the sample closest to baseline, which was defined as the time of blood culture order. Patients without a biobanked sample collected within 3 hours of a first blood culture order were excluded. Protein concentrations were measured using multiplexed Luminex MagPix assays developed by R&D Systems. Laboratory quality control procedures adhered strictly to the FDA Bioanalytical Method Validation Guidance for Industry to ensure data quality.

2,367 patients recruited from 3 hospital sites in the study were identified as triggerable by the trigger logic to serves as the data on which the algorithm was trained.
v. Input Feature Selection

The selected measurements used for the input features correspond to the logic specified in U.S. Provisional Patent Application No. 63/459,521, filed Apr. 14, 2023, which is incorporated by reference herein in its entirety. Imputation of measurements was performed with the same criteria specified elsewhere herein. During development, a surrogate for the order of the Sepsis ImmunoScore was replaced with the order of the first blood culture during the encounter.

vi. Training Label

The label used to train the Sepsis ImmunoScore Machine Learning Model was a derived Sepsis-3 label within 24 hours of the first blood culture order. The derived Sepsis-3 label was calculated by the presence of Organ Dysfunction, and Infection, in specific embodiments. The timing of the Organ Dysfunction event corresponds to the time of Sepsis-3, given the patient met the Infection criteria. Further details of the Organ Dysfunction component and the Infection criteria are listed below.

vii. Organ Dysfunction

Organ Dysfunction was calculated using available medications, laboratory results, vitals, and patient assessments present within the NOSIS Database during their encounter. Organ Dysfunction is calculated with the following equation:

SOFA−SOFAbaseline>=2

The SOFA score is calculated as the sum of the CNS, Cardiovascular, Respiratory, Renal, Coagulation, and Hepatic system SOFA components. The methods to calculate the SOFA score are identified below in Table 6. Baseline SOFA was calculated as the SOFA component in a prior encounter, if available. If no prior encounter was available, the baseline was assumed to be 0.

Table 6: SOFA Score components—The calculation of the SOFA Score components (Lambden, S., Laterre, P. F., Levy, M. M., & Francois, B. (2019). The SOFA score—development, utility and challenges of accurate assessment in clinical trials. Critical Care, 23(1), 374).

| | | | | |
|---|---|---|---|---|
| Respiration | <400 | <300 | <200 ------with | <100 |
| PaO₂/FiO₂′ | | | respiratory | support------- |
| mmHg | | | | |
| Coagulation | <150 | <100 | <50 | <20 |
| Platelets × 10₃/ | | | | |
| mm₃ | | | | |
| Liver | 1.2-1.9 | 2.0-5.9 | 6.0-11.9 | >12.0 |
| Bilirubin, mg/dl | (20-32) | (33-101) | (102-204) | (<204) |
| (μmol/l) | | | | |
| Cardiovascular | MAP <70 | Dopamine ≤5 or | Dopamine >5 or | Dopamine >15 or |
| Hypotension | mmHg | dobutamine | epinephrine ≤0.1 or | epinephrine ≤0.1 or |
| | | (any dose)^{a} | norepinephrine ≤0.1 | norepinephrine ≤0.1 |
| Central Nervous | 13-14 | 10-12 | 6-9 | <6 |
| System | | | | |
| Glasgow Coma | | | | |
| score | | | | |
| Renal | 1.2-1.9 | 2.0-3.4 | 3.5-4.9 | >5.0 |
| Creatinine, | (110-170) | (171-299) | (300-440) | (>440) |
| mg/dl | | | or <500 ml/day | or <200 ml/day |
| (μmol/l) or urine | | | | |
| Output | | | | |

^{a}Adrenergic agents administered for at least 1 h (doses given are in μg/kg · min)

viii. Infection

The infection criteria for Sepsis ImmunoScore is identified if one of the following criteria are met:
1. 4 or more Qualifying Antimicrobial Days (QAD) within the 2-day window around the first blood culture order
2. A positive microbiology test or culture except for known contaminants

The QAD rule follows the description in the CDC's Hospital Tool Kit for Adult Sepsis rules for infection (https://www.cdc.gov/sepsis/pdfs/Sepsis-Surveillance-Toolkit-Mar-2018_508.pdt). The positive microbiology is an addition to the CDC's Hospital Tool Kit for Adult Sepsis. The known contaminant list in blood cultures is found in Table 7.

Table 7: Known Blood Culture Contaminants—Table depicting known contaminants within blood cultures (Hall, Keri K., and Jason A. Lyman. “Updated Review of Blood Culture Contamination.” Clinical Microbiology Reviews, vol. 19, no. 4, 2006, pp. 788-802., https://doi.org/10.1128/cmr.00062-05). False-positive rates for organisms that frequently represent contamination

| | Source | | | |
|---|---|---|---|---|
| | 1,585 blood culture-positive | | 497,134 blood cultures from | |
| | episodes from 3 U.S. hospitals^{a} | | 640 U.S. institutions^{b} | |
| | % of all positive | Contamination | % of all positive | Contamination |
| Organism(s) | cultures (n = 1,585) | rate (%) | cultures | rate (%) |
| Coagulase- | 44.3 | 82 | Not reported | 62-63 |
| negative | | | | |
| staphylococci | | | | |
| Corynebacterium | 33.4 | 96 | Not reported | 68-78 |
| spp. (other than | | | | |
| C. jeikieum) | | | | |
| Bacillus spp. | 0.8 | 91.7 | Not reported | 68-70 (other than |
| | | | | B. anthracis) |
| Propionibacterium | 3.0 | 100 | Not reported | 84-85 |
| acnes | | | | |
| Viridans group | 4.5 | 49.3 | Not reported | 32-33 |
| streptococci | | | | |
| Clostridium | 0.8 | 76.9 | Not reported | Not reported |
| perfringens | | | | |

^{a}Data are from reference 155.
^{b}Data are from reference 113.

Known contaminants within urine culture are defined as “Lactobacilli, Corynebacteria species, Gardnerella, alphahaemolytic streptococci, and aerobes (Franz, Martina, and Walter H. Hörl. “Common Errors in Diagnosis and Management of Urinary Tract Infection. I: Pathophysiology and Diagnostic Techniques.” Nephrology Dialysis Transplantation, vol. 14, no. 11, 1999, pp. 2746-2753., https://doi.org/10.1093/ndt/14.11.2746).

ix. Hyperparameters

In particular embodiments, the hyperparameters associated with the Random Forest model are the mtry, minimum node size, and split rule. Various combinations of mtry, minimum node size, and the split rule were considered. In certain embodiments, the combination that provided the best 5-fold cross-validation AUROC results with 3 repeats was selected as the optimal hyperparameters. The R package ‘caret’ was used to facilitate optimal hyperparameter selection.

x. Performance Evaluation

In certain embodiments, the pre-trained machine learning model is evaluated by assessing the diagnostic performance and prognostic performance. Both performances are evaluated using the calculated sepsis risk scores and the risk thresholds identified during the development of the Sepsis ImmunoScore Algorithm.

xi. Risk Threshold Creation

A receiver operating characteristic (ROC) curve was generated using the uncalibrated predictions against the training label during the development of the Sepsis ImmunoScore Algorithm. This ROC curve was used to identify three thresholds with the following criteria, in certain embodiments:
1. Lowest Threshold—Identifying the threshold with the highest sensitivity whilst maintaining a false positive rate of 50%.
2. Optimal Threshold—Threshold is identified as the ‘top-left’ point of the ROC curve, i.e., the point on the ROC curve with minimal Euclidean distance to (0,1).
3. 95th percentile threshold—Threshold corresponding to the 95th percentile of sepsis risk scores within the training dataset.
   xii. Diagnostic Performance

The global machine learning model diagnostic performance was measured by identifying the global area under the OOB AUROC and the area under the OOB Precision-Recall Curve (AURPC). Furthermore, the diagnostic metrics (ex: sensitivity, specificity, positive-predictive-value, negative-predictive-value, etc.) were evaluated at each threshold.

xiii. Prognostic Performance

The prognostic performance was evaluated by identifying the prevalence of the adverse events identified elsewhere herein for each of the four risk categories.

xiv. Calibration Model

Classifiers generated by random forest models, despite being both accurate and achieving a high AUC, produce class probabilities that are of rather poor quality, as measured by the squared error of the predicted probabilities (Brier score). One approach to addressing this problem is by means of a calibration model, i.e., learning a function that maps the original probability estimates, or scores, into more accurate probability estimates.

In particular embodiments, the Calibration Model was generated by performing a Platt calibration. Platt calibration can be created by training a logistic regression model with the pre-calibrated Sepsis ImmunoScore to predict the Sepsis-3 training label. The calibration model may be assessed by identifying the percentage of septic patients given the Sepsis ImmunoScore.

### IV. Exemplary Methodologies

### IV.A. Exemplary Methodology

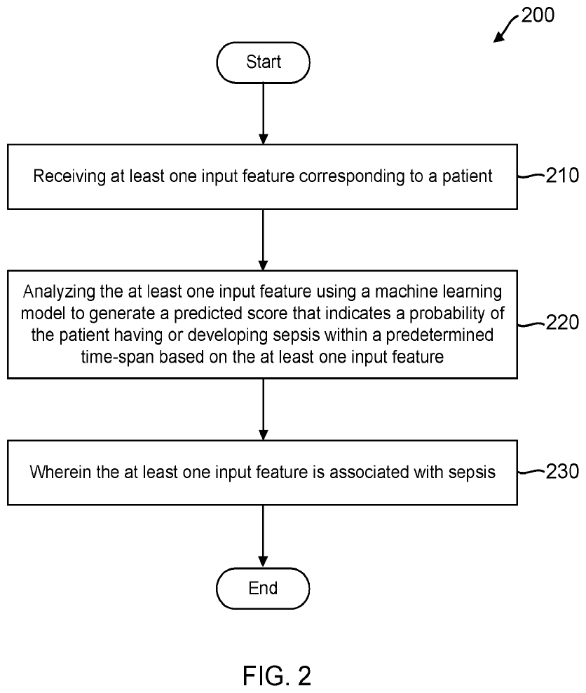

FIG. 2 illustrates an example method 200 for generating an indicator of risk for sepsis, in accordance with various embodiments. At step 210 of method 200, the method includes the step of receiving at least one input feature corresponding to a patient. At step 220, the method further includes the step of analyzing the at least one input feature using a machine learning model to generate a predicted score that indicates a probability of the patient having or developing sepsis within a predetermined time-span based on the at least one input feature. In accordance with various embodiments, optionally (step 230), the at least one input feature is associated with sepsis.

In accordance with various embodiments, the at least one input feature can be divided into two groups. These two groups can be concentration values, wherein a first group of concentration values has a lower relative significance than a second group of concentration values.

In accordance with various embodiments, the at least one input feature comprises Procalcitonin and can be divided into two groups. These two groups can be Procalcitonin concentration values, wherein a first group of Procalcitonin concentration values has a lower relative significance than a second group of Procalcitonin concentration values.

In accordance with various embodiments, the at least one input feature can be divided into three groups. These three groups can be concentration values, wherein a first group of concentration values has a lower relative significance than a second group of concentration values, and wherein a third group of concentration values has a higher relative significance than both the first group and second group of concentration values.

In accordance with various embodiments, the at least one input feature comprises Procalcitonin and can be divided into three groups. These three groups can be Procalcitonin concentration values, wherein a first group of Procalcitonin concentration values has a lower relative significance than a second group of Procalcitonin concentration values, and wherein a third group of Procalcitonin concentration values has a higher relative significance than both the first group and second group of Procalcitonin concentration values.

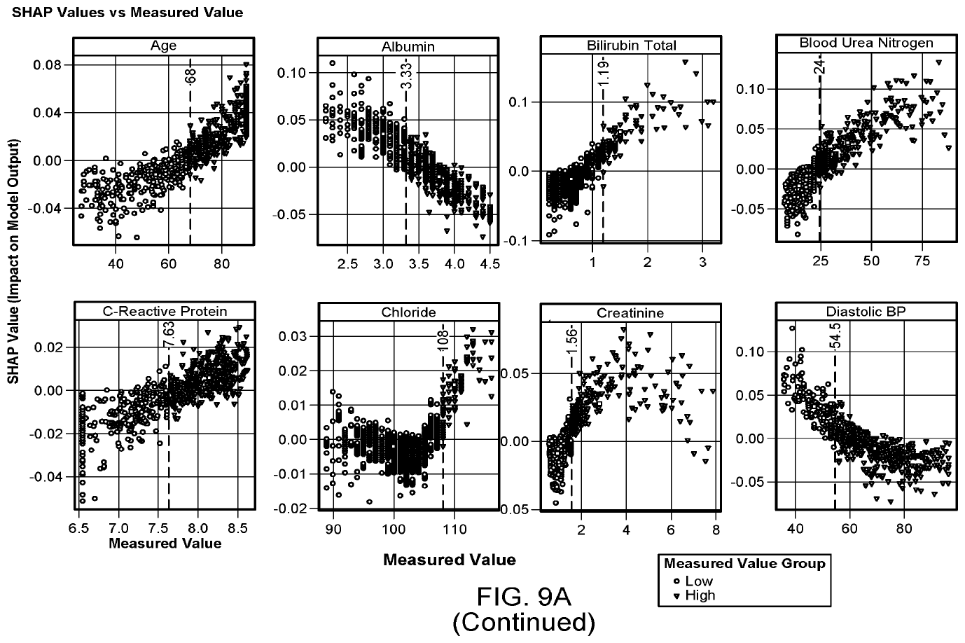

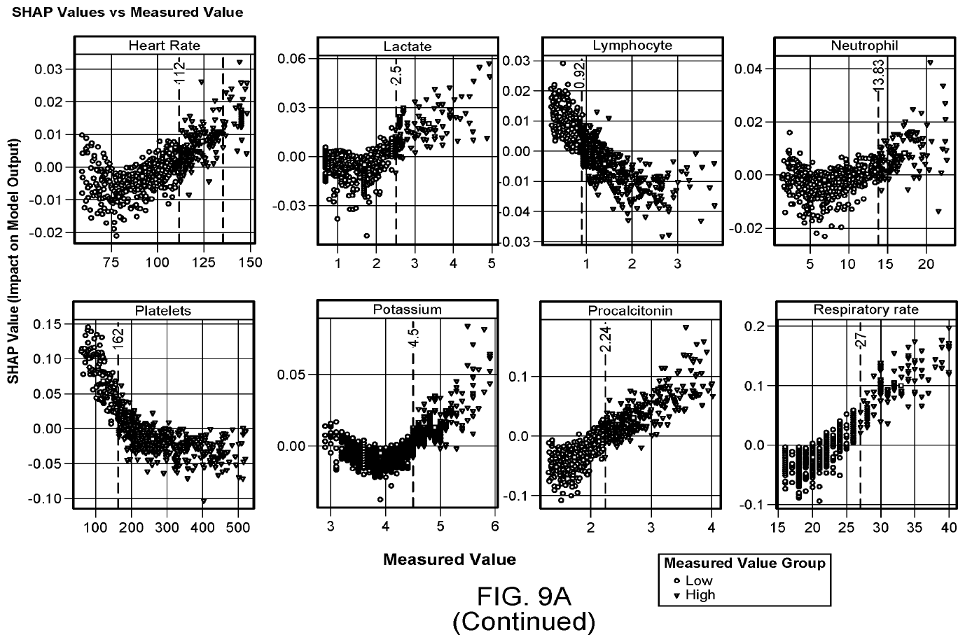

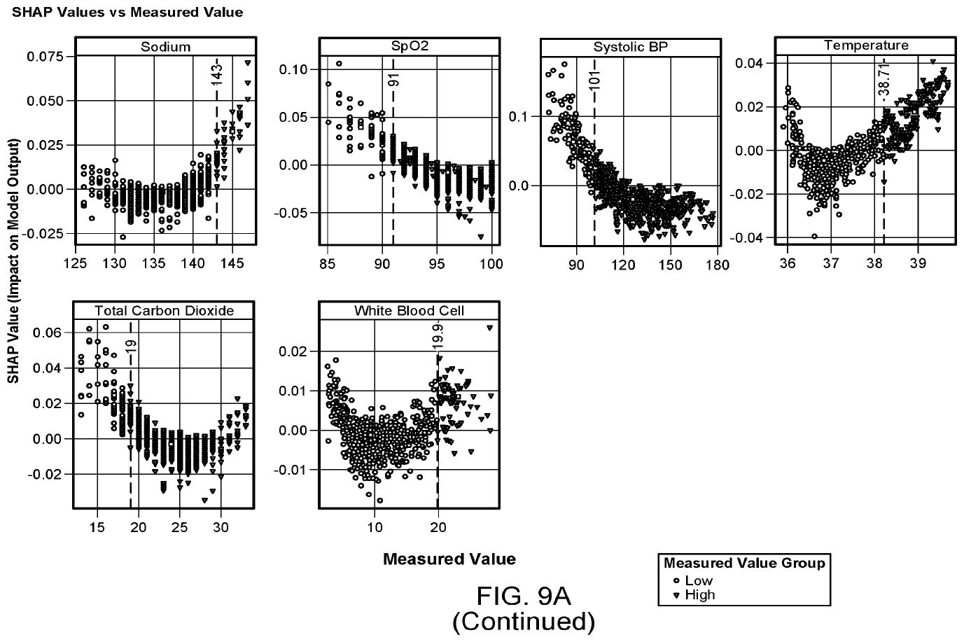

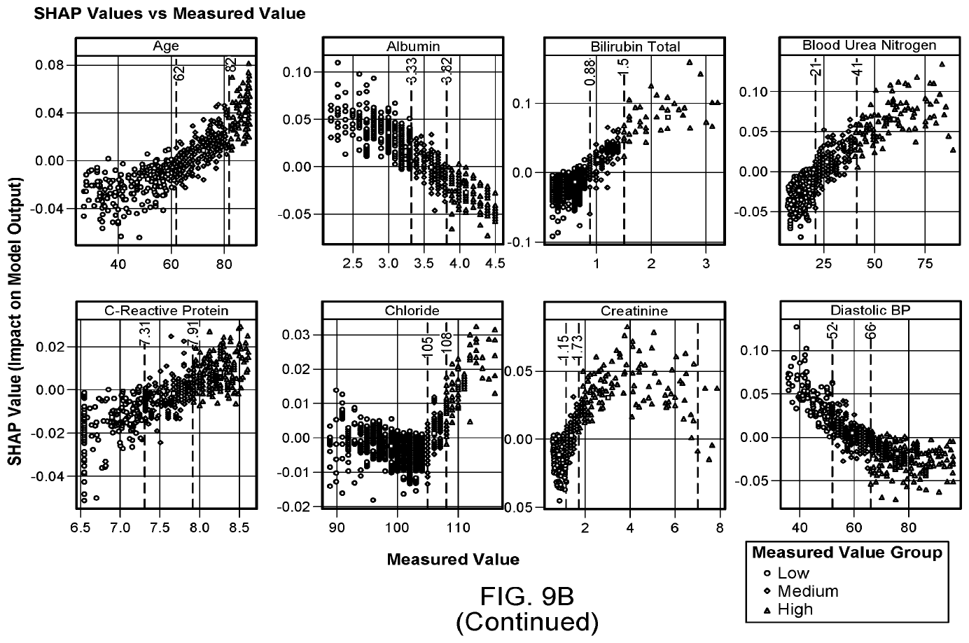

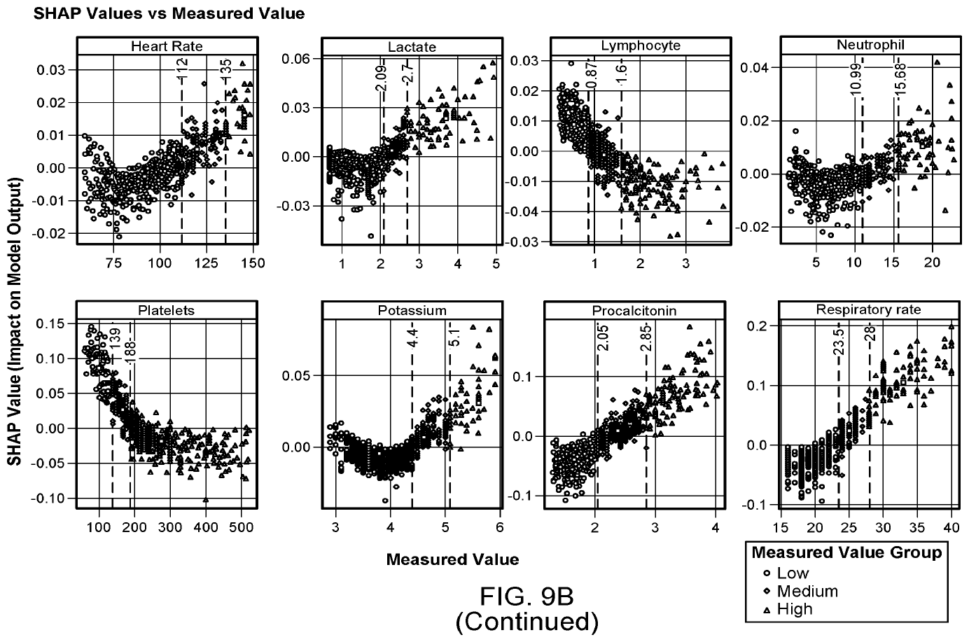

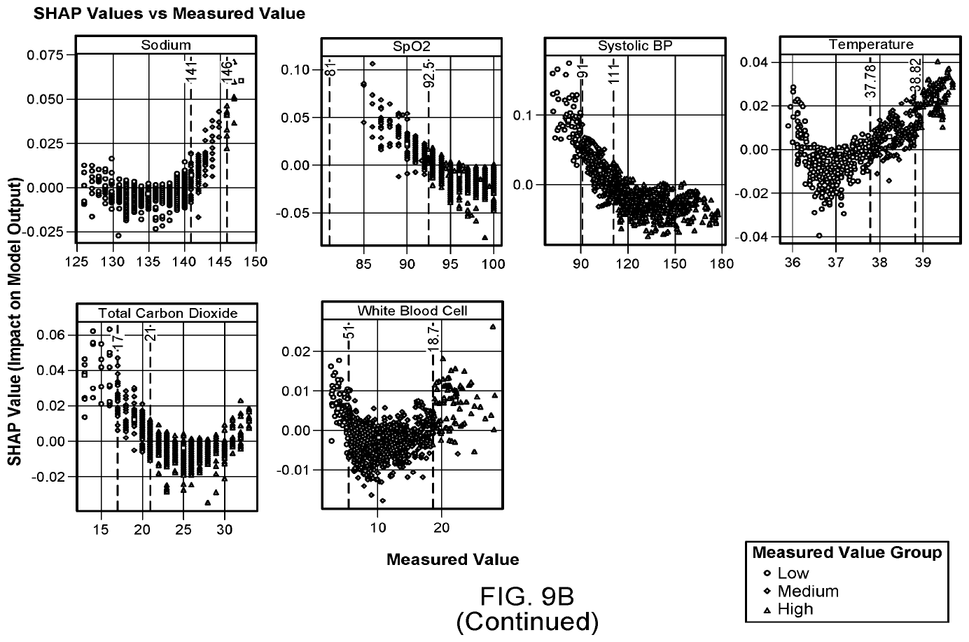

FIG. 9A illustrates a non-limiting example of a 2-group, or bin, feature applied to Model 1A (discussed in detail below). FIG. 9B illustrates a non-limiting example of a 3-group, or bin, feature applied to Model 1A.

This example method can be further modified, for example, as provided in claims 2-171, in accordance with various embodiments. The additional features contained therein can be implemented in any combination as desired.

### V. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Examples of Algorithm Results and Feature Importance Rankings for Exemplary Models

The present Example provides examples of algorithm results and feature importance rankings for multiple versions of a Sepsis algorithm. The versions differ in the set of input features used to predict sepsis. In particular embodiments, six different models utilize a variety of one or more Demographic Measurements, one or more Patient Assessments, one or more Vital Signs, one or more Hematology Laboratory Values, one or more Chemistry Laboratory Values, and/or one or more Sepsis-associated biomarker concentrations.

### V.A. Model 1A

Exemplary input features for Model 1A are provided below and in no particular order.

TABLE 8
Examples of Model 1A Input Features

Age
Albumin
Bilirubin
BUN
Chloride
CO₂
C-Reactive Protein
Creatinine
Diastolic Blood Pressure
Heart Rate
Lactate
Lymphocytes
Neutrophil
Platelets
Potassium
Procalcitonin
Pulse Oximetry
Respiratory Rate
Sodium
Systolic Blood Pressure
Temperature
White Blood Cell Count

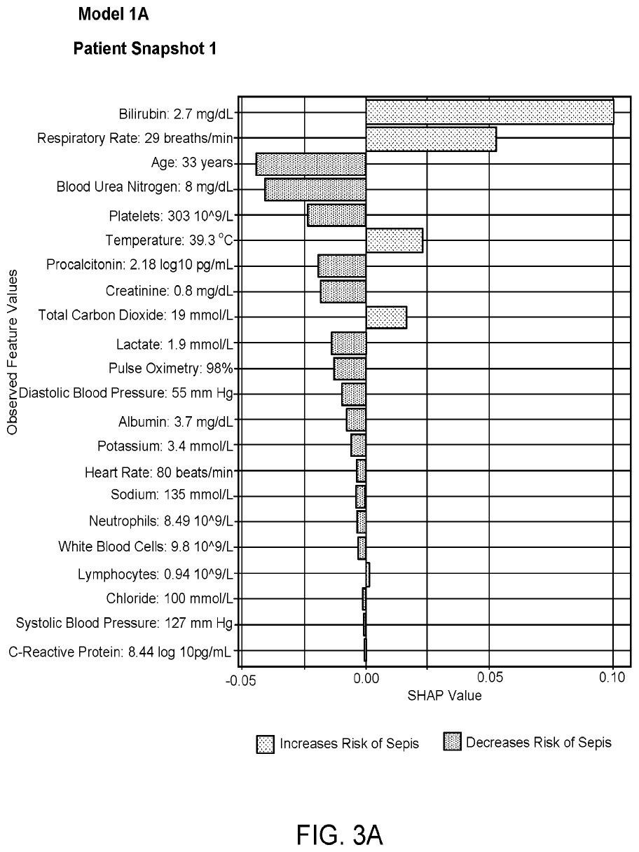

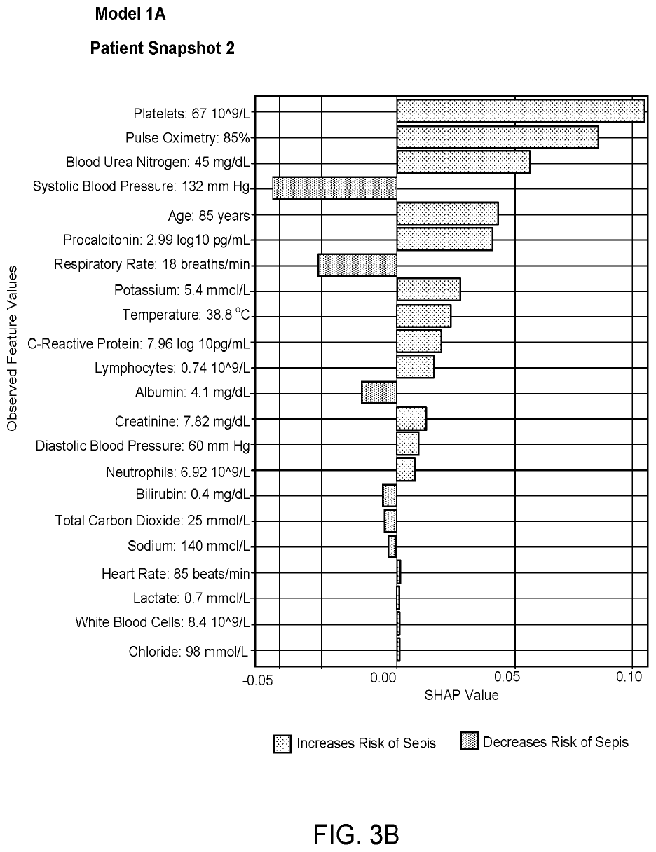

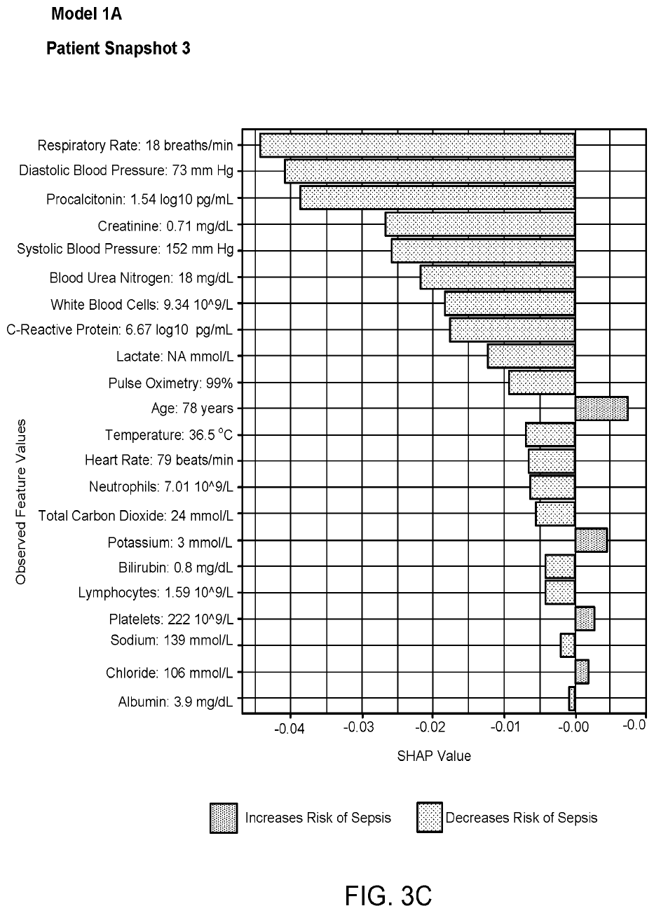

In one embodiment for Model 1A, an example of SHAP values for three different patients at risk for sepsis is provided in FIG. 3A (not septic within 24 hours), FIG. 3B (septic within 24 hours), and FIG. 3C (not septic within 24 hours). The order of importance of the input features is different among the three patients.

FIG. 3A illustrates a snapshot of the Patient 1 SHAP values for the noted input features. Most of the feature values for this patient have negative SHAP values, i.e., reduce their Sepsis ImmunoScore, especially their young age and their normal BUN, platelet and creatinine lab values, which give no indication of dysfunction in these organs. However, their elevated bilirubin indicates abnormal liver function, and their elevated respiratory rate and to a lesser extent temperature do have substantially positive SHAP values.

FIG. 3B illustrates a snapshot of the Patient 2 SHAP values for the noted input features. This elderly patient has positive SHAP values for most features, with the greatest contributions from platelets, low pulse ox, and BUN values that indicate organ dysfunction consistent with the Sepsis-3 definition of organ dysfunction (an increase in SOFA score of at least 2 from baseline).

FIG. 3C illustrates a snapshot of the Patient 2 SHAP values for the noted input features. This patient has negative SHAP values for nearly all features, with the exception of age and a few miscellaneous lab values that are marginally positive. Both organ function and infection related features are represented in the most significant SHAP values, all in negative direction. This patient is unlikely to become septic within the next 24 hours.

Exemplary results for Model 1A diagnostic performance and prognostic performance are as follows:

Diagnostic Performance

| Risk | | | | Likelihood |
|---|---|---|---|---|
| Group | Septic | Not Septic | Percentage Septic | Ratio |
| Low | 16 | 215 | 6.93% [4.86%, | 0.20 [0.14, |
| | | | 9.58%] | 0.27] |
| Medium | 30 | 150 | 16.67% [13.12%, | 0.54 [0.42, |
| | | | 20.78%] | 0.67] |
| High | 125 | 163 | 43.40% [39.53%, | 2.07 [1.85, |
| | | | 47.34%] | 2.30] |
| Very | 28 | 8 | 77.78% [66.42%, | 9.43 [5.77, |
| High | | | 86.61%] | 17.21] |

Prognostic Performance

| | In- | | | Ventilated | Vasopressors |
|---|---|---|---|---|---|
| Risk | Hospital | Length | ICU within | within 24 | within 24 |
| Group | Mortality | of Stay | 24 Hours | Hours | Hours |
| Low | 0.43% | 3.91 | 8.66% | 1.73% | 0.87% |
| | [0.05%, | [3.56, | [6.35%, | [0.76%, | [0.23%, |
| | 1.67%] | 4.63] | 11.53%] | 3.43%] | 2.29%] |
| Medium | 3.89% | 6.11 | 21.67% | 4.44% | 2.78% |
| | [2.18%, | [5.18, | [17.71%, | [2.60%, | [1.36%, |
| | 6.45%] | 7.24] | 26.11%] | 7.12%] | 5.09%] |
| High | 10.07% | 7.50 | 36.46% | 7.99% | 12.15% |
| | [7.84%, | [6.59, | [32.73%, | [5.99%, | [9.72%, |
| | 12.73%] | 8.91] | 40.33%] | 10.44%] | 14.99%] |
| Very | 30.56% | 15.16 | 61.11% | 19.44% | 38.89% |
| High | [20.42%, | [10.14, | [49.05%, | [11.16%, | [27.82%, |
| | 42.44%] | 22.17] | 72.18%] | 30.53%] | 50.95%] |

### V.B. Model 1B

Exemplary input features for Model 1B are provided below and in no particular order. In particular embodiments, Model 1B input features include IL-6 that may be lacking in the input features for Model 1A.

TABLE 9
Examples of Model 1B Input Features

Age
Albumin
Bilirubin
BUN
Chloride
CO₂
C-Reactive Protein
Creatinine
Diastolic Blood Pressure
Heart Rate
IL-6
Lactate
Lymphocytes
Neutrophil
Platelets
Potassium
Procalcitonin
Pulse Oximetry
Respiratory Rate
Sodium
Systolic Blood Pressure
Temperature
White Blood Cell Count

Exemplary results for Model 1B diagnostic performance and prognostic performance are as follows:

| Risk | | | | Likelihood |
|---|---|---|---|---|
| Group | Septic | Not Septic | Percentage Septic | Ratio |
| Low | 17 | 205 | 7.66% [5.44%, | 0.22 [0.15, |
| | | | 10.47%] | 0.28] |
| Medium | 26 | 147 | 15.03% [11.58%, | 0.46 [0.35, |
| | | | 19.11%] | 0.59] |
| High | 124 | 162 | 43.36% [39.47%, | 2.00 [1.80, |
| | | | 47.31%] | 2.24] |
| Very | 32 | 6 | 84.21% [73.93%, | 13.94 [8.55, |
| High | | | 91.49%] | 29.09] |

### Prognostic Performance

| | In- | | | Ventilated | Vasopressors |
|---|---|---|---|---|---|
| Risk | Hospital | Length | ICU within | within 24 | within 24 |
| Group | Mortality | of Stay | 24 Hours | Hours | Hours |
| Low | 0.45% | 3.92 | 9.01% | 1.80% | 1.35% |
| | [0.05%, | [3.60, | [6.61%, | [0.79%, | [0.50%, |
| | 1.74%] | 4.71] | 11.99%] | 3.57%] | 2.98%] |
| Medium | 5.20% | 5.78 | 21.39% | 4.62% | 2.89% |
| | [3.16%, | [4.77, | [17.37%, | [2.71%, | [1.41%, |
| | 8.09%] | 6.99] | 25.91%] | 7.40%] | 5.29%] |
| High | 8.74% | 7.27 | 36.36% | 6.99% | 11.19% |
| | [6.65%, | [6.36, | [32.62%, | [5.12%, | [8.84%, |
| | 11.28%] | 8.53] | 40.25%] | 9.34%] | 13.96%] |
| Very | 34.21% | 18.78 | 65.79% | 26.32% | 42.11% |
| High | [23.91%, | [13.33, | [54.15%, | [17.04%, | [31.08%, |
| | 45.85%] | NA] | 76.09%] | 37.62%] | 53.79%] |

### V.C. Model 2

Exemplary input features for Model 2 are provided below and in no particular order.

TABLE 10
Examples of Model 2 Input Features

Age
Albumin
Angiopoietin-2
Bilirubin
BUN
Chloride
CO2
C-Reactive Protein
Creatinine
Diastolic Blood Pressure
Heart Rate
IL-6
IL-8
IP-10
Lactate
Lymphocytes
Neutrophil
Pentraxin-3
Platelets
Potassium
Procalcitonin
Pulse Oximetry
Respiratory Rate
Sodium
Systolic Blood Pressure
Temperature
White Blood Cell Count

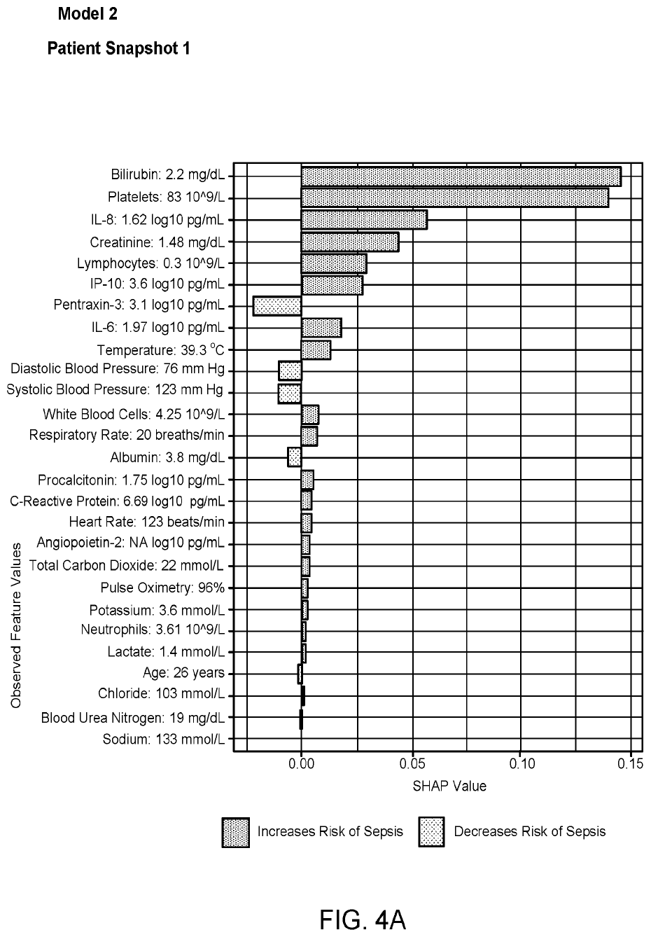

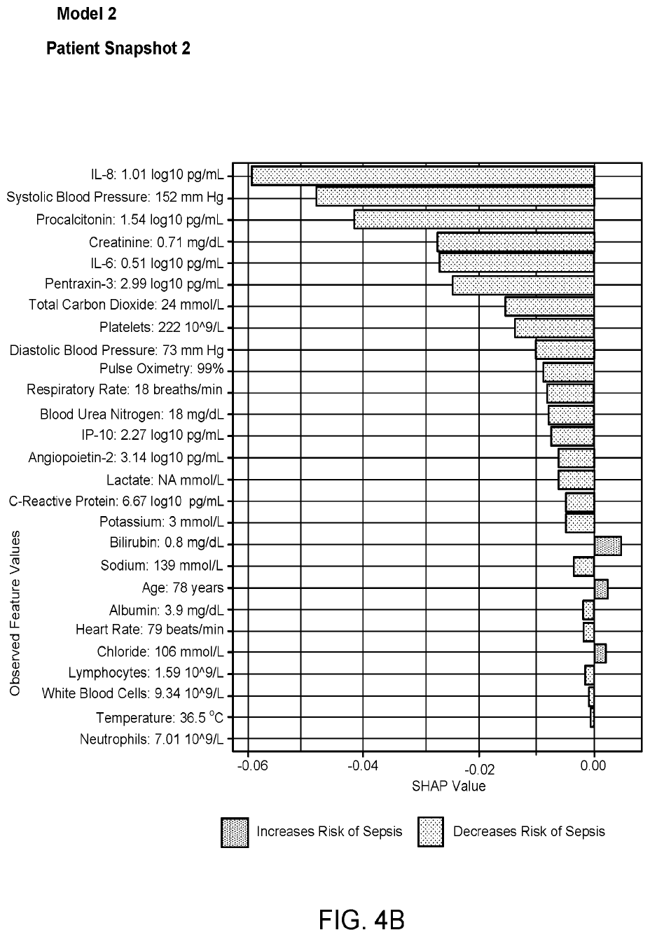

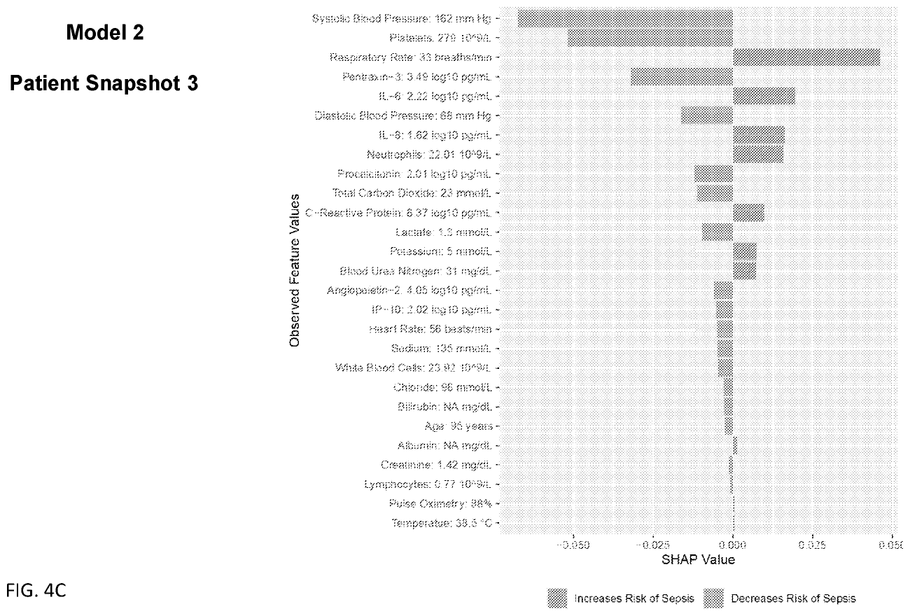

In one embodiment for Model 2, an example of SHAP values for three different patients at risk for sepsis is provided in FIG. 4A (septic within 24 hours), FIG. 4B (septic within 24 hours), and FIG. 4C (not septic within 24 hours). The input features have different ordering of importance dependent on the patient.

FIG. 4A illustrates a snapshot of the Patient 1 SHAP values for the noted input features. SHAP values for most features are positive for this patient, led by their bilirubin and platelets values, both of which indicate organ dysfunction. Their feature values for a number of infection-related markers suggest this organ dysfunction may be due to infection, which is the definition of sepsis-3.

FIG. 4B illustrates a snapshot of the Patient 2 SHAP values for the noted input features. This patient has almost universally negative SHAP values, corresponding to a very low Sepsis ImmunoScore. Many of the most significant (i.e., largest in magnitude) of these are protein biomarkers, including IL-8, Procalcitonin, IL-6 and Pentraxin-3.

FIG. 4C illustrates a snapshot of the Patient 3 SHAP values for the noted input features. This patient has both positive and negative SHAP values of appreciable magnitude, including elevated respiratory rate and certain sepsis-related biomarkers like IL-6, IL-8, and CRP. However, their normal blood pressure and platelet numbers have the overall strongest contribution.

Exemplary results for Model 2 diagnostic performance and prognostic performance are as follows:

### Diagnostic Performance

| Risk | | | | Likelihood |
|---|---|---|---|---|
| Group | Septic | Not Septic | Percentage Septic | Ratio |
| Low | 12 | 199 | 5.69% [3.74%, | 0.16 [0.11, |
| | | | 8.31%] | 0.23] |
| Medium | 24 | 142 | 14.46% [11.00%, | 0.46 [0.35, |
| | | | 18.58%] | 0.58] |
| High | 132 | 195 | 40.37% [36.79%, | 1.84 [1.67, |
| | | | 44.03%] | 2.02] |
| Very | 33 | 9 | 78.57% [68.29%, | 9.94 [6.49, |
| High | | | 86.63%] | 17.40] |

### Prognostic Performance

| | In- | | | Ventilated | Vasopressors |
|---|---|---|---|---|---|
| Risk | Hospital | Length | ICU within | within 24 | within 24 |
| Group | Mortality | of Stay | 24 Hours | Hours | Hours |
| Low | 0.47% | 3.91 | 9.00% | 1.90% | 0.47% |
| | [0.05%, | [3.70, | [6.55%, | [0.83%, | [0.05%, |
| | 1.83%] | 4.71] | 12.07%] | 3.75%] | 1.83%] |
| Medium | 4.22% | 5.72 | 21.69% | 3.61% | 3.01% |
| | [2.36%, | [4.83, | [17.56%, | [1.91%, | [1.47%, |
| | 6.99%] | 6.96] | 26.34%] | 6.26%] | 5.52%] |
| High | 8.87% | 7.21 | 33.94% | 7.34% | 11.01% |
| | [6.90%, | [6.49, | [30.52%, | [5.54%, | [8.82%, |
| | 11.23%] | 8.52] | 37.52%] | 9.55%] | 13.56%] |
| Very | 30.95% | 14.26 | 57.14% | 19.05% | 33.33% |
| High | [21.52%, | [9.55, | [46.09%, | [11.42%, | [23.63%, |
| | 41.84%] | 22.17] | 67.66%] | 29.09%] | 44.31%] |

### V.D. Model 3

Exemplary input features for Model 3 are provided below and in no particular order.

TABLE 11
Examples of Model 3 Input Features

Age
Albumin
Angiopoietin-2
Bilirubin
BUN
Chloride
CO2
C-Reactive Protein
Creatinine
Diastolic Blood
Pressure
Heart Rate
IL-6
IL-8
IP-10
Lactate
Lymphocytes
Neutrophil
PDL-1
Pentraxin-3
Platelets
Potassium
Procalcitonin
Pulse Oximetry
Respiratory Rate
Sodium
Systolic Blood
Pressure
Temperature
TREM-1
White Blood Cell
Count

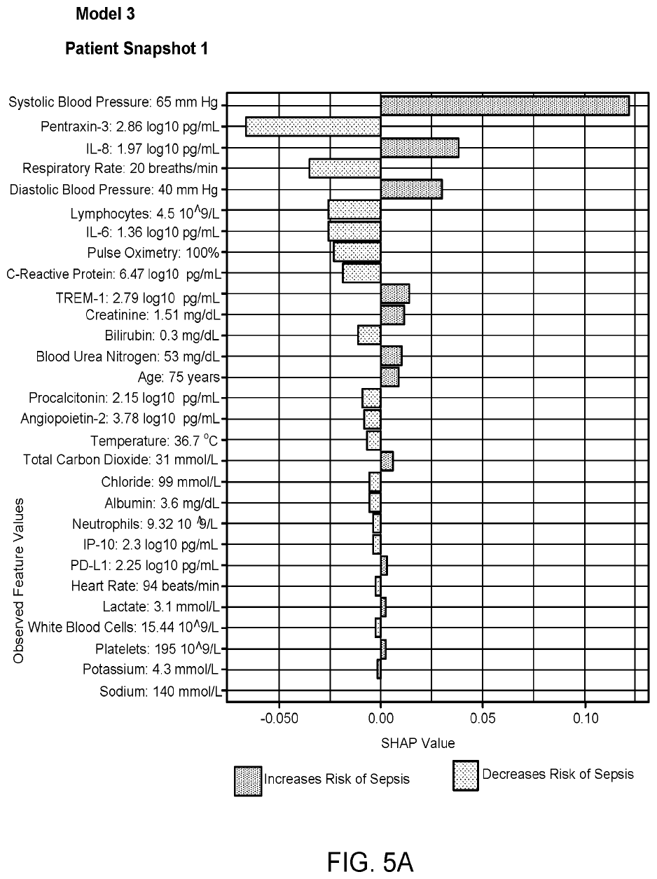

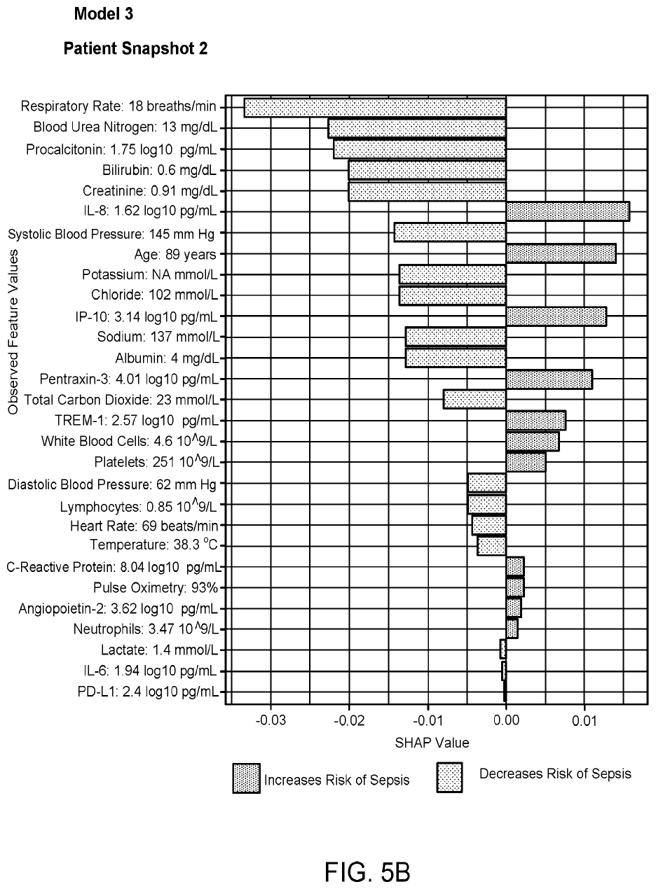

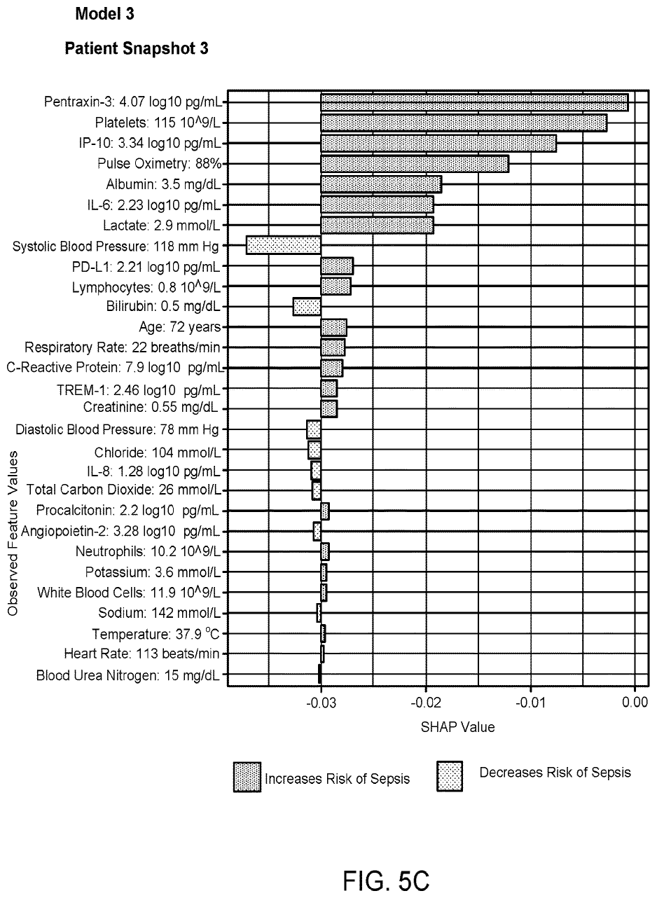

In one embodiment for Model 3, an example of SHAP values for three different patients at risk for sepsis is provided in FIG. 5A (not septic within 24 hours), FIG. 5B (not septic within 24 hours), and FIG. 5C (septic within 24 hours). The order of importance of the input features is different among the three patients.

FIG. 5A illustrates a snapshot of the Patient 1 SHAP values for the noted input features. This patient has both highly positive and highly negative SHAP values. Abnormal blood pressure readings and elevated creatinine and several protein biomarkers (e.g., IL-8, TREM-1) all increase their Sepsis ImmunoScore. However, other biomarkers, including Pentraxin-3, IL-6 and CRP as well as their respiratory rate and normal blood oxygenation reduce their risk score.

FIG. 5B illustrates a snapshot of the Patient 2 SHAP values for the noted input features. Normal respiratory rate and traditional labs like BUN, bilirubin and creatinine all provide substantially negative contributions to this patient's Sepsis ImmunoScore, outweighing elevated IL-8, IP-10 and advanced age.

FIG. 5C illustrates a snapshot of the Patient 3 SHAP values for the noted input features. Pentraxin-3 has the highest SHAP value for this patient along with other infection-related markers and abnormal values for measures of organ dysfunction (platelets, pulse oximetry).

Exemplary results for Model 3 diagnostic performance and prognostic performance are as follows:

### Diagnostic Performance

| Risk | | | | Likelihood |
|---|---|---|---|---|
| Group | Septic | Not Septic | Percentage Septic | Ratio |
| Low | 11 | 192 | 5.42% [3.48%, | 0.16 [0.10, |
| | | | 8.06%] | 0.21] |
| Medium | 26 | 151 | 14.69% [11.31%, | 0.47 [0.35, |
| | | | 18.68%] | 0.59] |
| High | 133 | 199 | 40.06% [36.52%, | 1.81 [1.65, |
| | | | 43.69%] | 2.00] |
| Very | 31 | 3 | 91.18% [81.40%, | 28.02 [15.84, |
| High | | | 96.71%] | 90.23] |

### Prognostic Performance

| | | | | Ventilated | Vasopressors |
|---|---|---|---|---|---|
| Risk | In-Hospital | Length | ICU within | within 24 | within 24 |
| Group | Mortality | of Stay | 24 Hours | Hours | Hours |
| Low | 0.49% | 3.88 | 10.34% | 2.46% | 0.49% |
| | [0.05%, | [3.46, | [7.67%, | [1.20%, | [0.05%, |
| | 1.90%] | 4.55] | 13.64%] | 4.52%] | 1.90%] |
| Medium | 3.95% | 5.35 | 19.21% | 2.82% | 3.39% |
| | [2.21%, | [4.77, | [15.41%, | [1.38%, | [1.79%, |
| | 6.56%] | 6.98] | 23.54%] | 5.18%] | 5.87%] |
| High | 8.73% | 7.40 | 34.34% | 6.93% | 9.94% |
| | [6.79%, | [6.49, | [30.92%, | [5.19%, | [7.87%, |
| | 11.06%] | 8.53] | 37.89%] | 9.07%] | 12.38%] |
| Very | 38.24% | 19.29 | 61.76% | 26.47% | 47.06% |
| High | [26.90%, | [14.41, | [49.32%, | [16.65%, | [35.03%, |
| | 50.68%] | NA] | 73.10%] | 38.55%] | 59.36%] |

### V.E. Model 4

Exemplary input features for Model 4 are provided below and in no particular order.

TABLE 12
Examples of Model 4 Input Features

Age
Albumin
Angiopoietin-1
Angiopoietin-2
Bilirubin
BUN
Chloride
CO₂
C-Reactive Protein
Creatinine
Diastolic Blood Pressure
E-Selectin
FLT3-Ligand
Fractalkine
GCSF
GMCSF
Granzyme-B
Heart Rate
IFN-alpha
IFN-gamma
IL-10
IL-15
IL1-beta
IL1-RA
IL-2
IL-4
IL-6
IL-7
IL-8
IP-10
Lactate
Leptin
Lymphocytes
MCP1
MIP1-alpha
MIP1-beta
MIP3-alpha
Neutrophil
NGAL
PDL-1
Pentraxin-3
Platelets
Potassium
Procalcitonin
Pulse Oximetry
Respiratory Rate
Sodium
Systolic Blood Pressure
Temperature
TGF-alpha
Thromodulin
Tissue Factor
TNF-alpha
TRAIL
TREM-1
VCAM-1
VEG-F
White Blood Cell Count

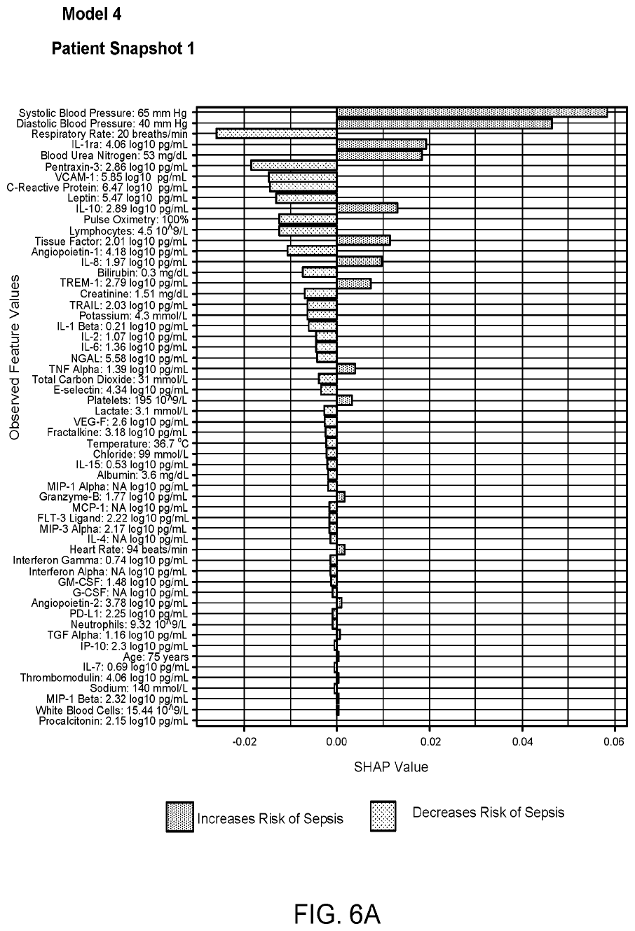

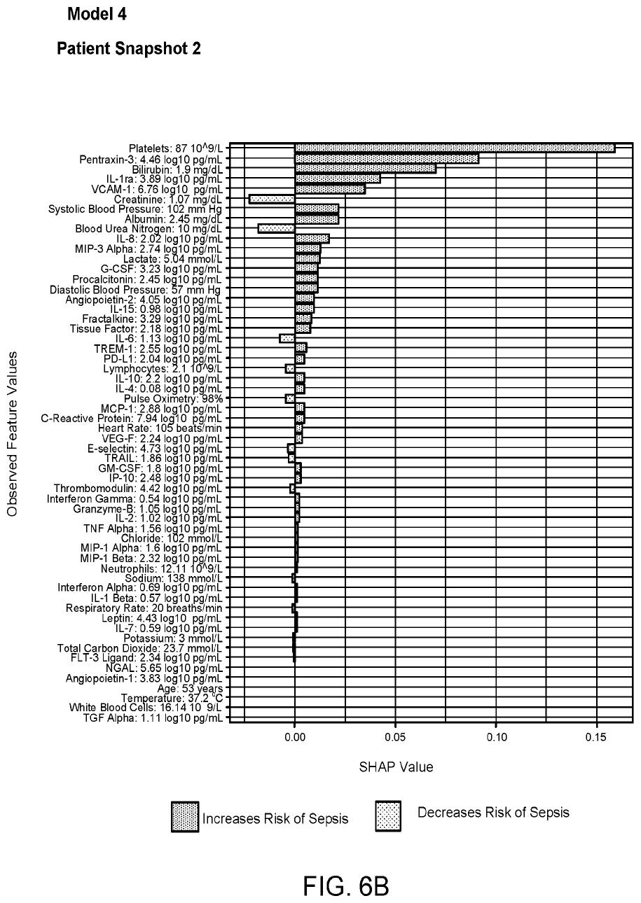

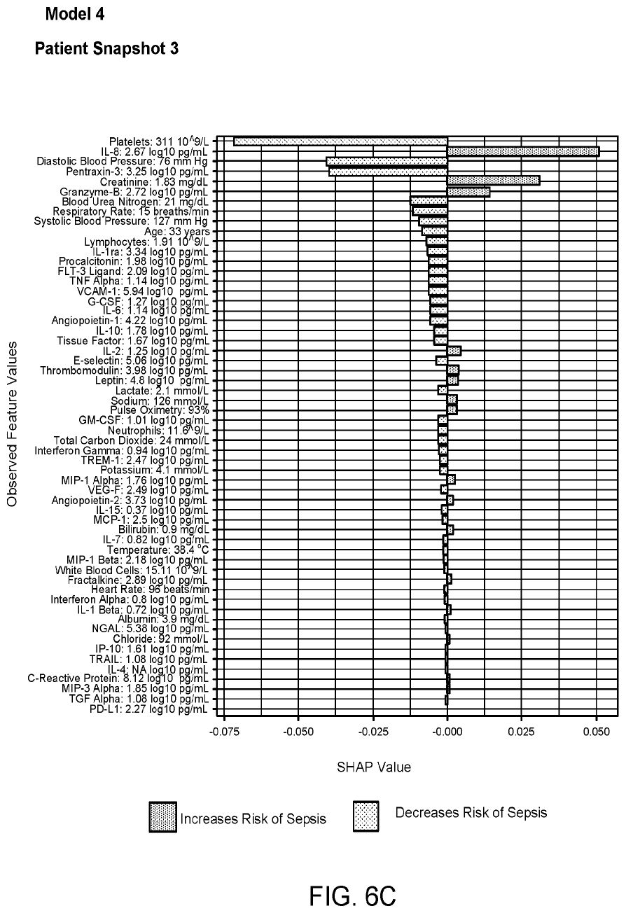

In one embodiment for Model 4, an example of SHAP values for three different patients at risk for sepsis is provided in FIG. 6A (not septic within 24 hours), FIG. 6B (septic within 24 hours), and FIG. 6C (not septic within 24 hours). The patients each have different rankings of input features.

FIG. 6A illustrates a snapshot of the Patient 1 SHAP values for the noted input features. Abnormal blood pressure indicating cardiovascular dysfunction made this patient's blood pressure readings their most significant SHAP values.

FIG. 6B illustrates a snapshot of the Patient 2 SHAP values for the noted input features. A low platelet count is the most important feature for this patient, as that qualifies them for coagulopathic organ dysfunction.

FIG. 6C illustrates a snapshot of the Patient 3 SHAP values for the noted input features. Elevated creatinine, IL-8 and Granzyme-B have positive SHAP values, but normal platelets, blood pressure and other feature values all contribute negatively to this patients risk of sepsis.

Exemplary results for Model 4 diagnostic performance and prognostic performance are as follows:

### Diagnostic Performance

| Risk | | | | Likelihood |
|---|---|---|---|---|
| Group | Septic | Not Septic | Percentage Septic | Ratio |
| Low | 10 | 197 | 4.83% [3.03%, | 0.14 [0.08, |
| | | | 7.35%] | 0.20] |
| Medium | 37 | 179 | 17.13% [13.85%, | 0.56 [0.45, |
| | | | 20.87%] | 0.68] |
| High | 126 | 164 | 43.45% [39.59%, | 2.08 [1.87, |
| | | | 47.38%] | 2.33] |
| Very | 28 | 5 | 84.85% [73.70%, | 15.18 [8.88, |
| High | | | 92.44%] | 33.76] |

### Prognostic Performance

| | In- | | | Ventilated | Vasopressors |
|---|---|---|---|---|---|
| Risk | Hospital | Length | ICU within | within 24 | within 24 |
| Group | Mortality | of Stay | 24 Hours | Hours | Hours |
| Low | 0.48% | 3.93 | 9.18% | 1.93% | 0.97% |
| | [0.05%, | [3.73, | [6.68%, | [0.85%, | [0.26%, |
| | 1.87%] | 4.75] | 12.30%] | 3.82%] | 2.55%] |
| Medium | 5.09% | 5.02 | 21.30% | 3.24% | 2.31% |
| | [3.27%, | [4.80, | [17.71%, | [1.81%, | [1.13%, |
| | 7.58%] | 6.35] | 25.29%] | 5.39%] | 4.25%] |
| High | 9.66% | 7.61 | 36.90% | 8.62% | 13.10% |
| | [7.48%, | [6.75, | [33.17%, | [6.56%, | [10.59%, |
| | 12.27%] | 8.79] | 40.76%] | 11.13%] | 16.01%] |
| Very | 30.30% | 14.33 | 54.55% | 18.18% | 33.33% |
| High | [19.75%, | [10.14, | [41.99%, | [9.84%, | [22.38%, |
| | 42.79%] | NA] | 66.66%] | 29.73%] | 45.92%] |

### V.F. Model 5

Exemplary input features for Model 5 are provided below and in no particular order.

TABLE 13
Examples of Model 5 Input Features

Age
Angiopoietin-2
BUN
Calcium
Chloride
CO₂
Creatinine
Diastolic Blood Pressure
Female
Glucose
Heart Rate
Hematocrit
Hemoglobin
IL-8
IP-10
Pentraxin-3
Potassium
Pulse Oximetry
Respiratory Rate
Sodium
Systolic Blood Pressure
Temperature

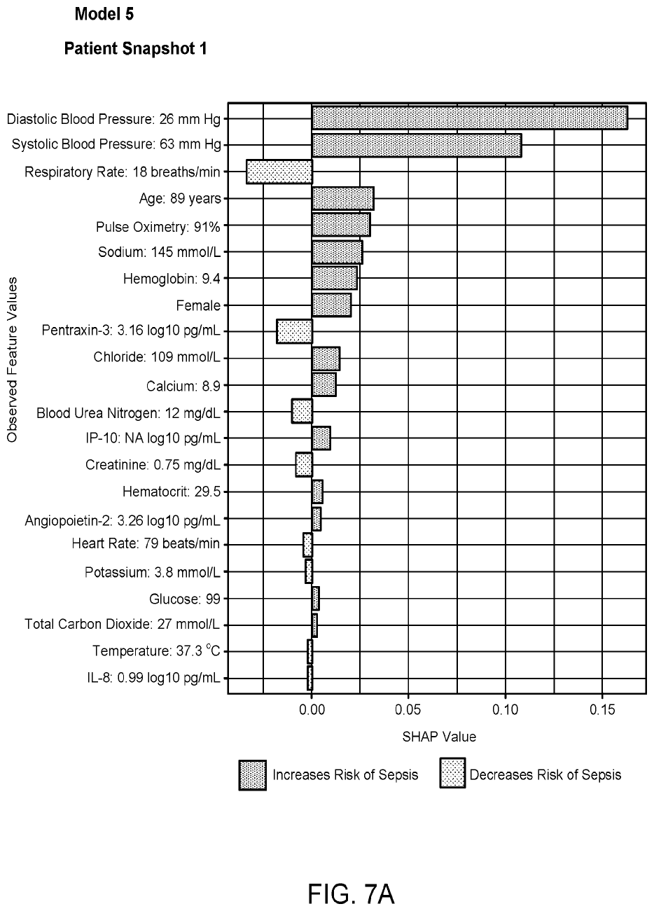

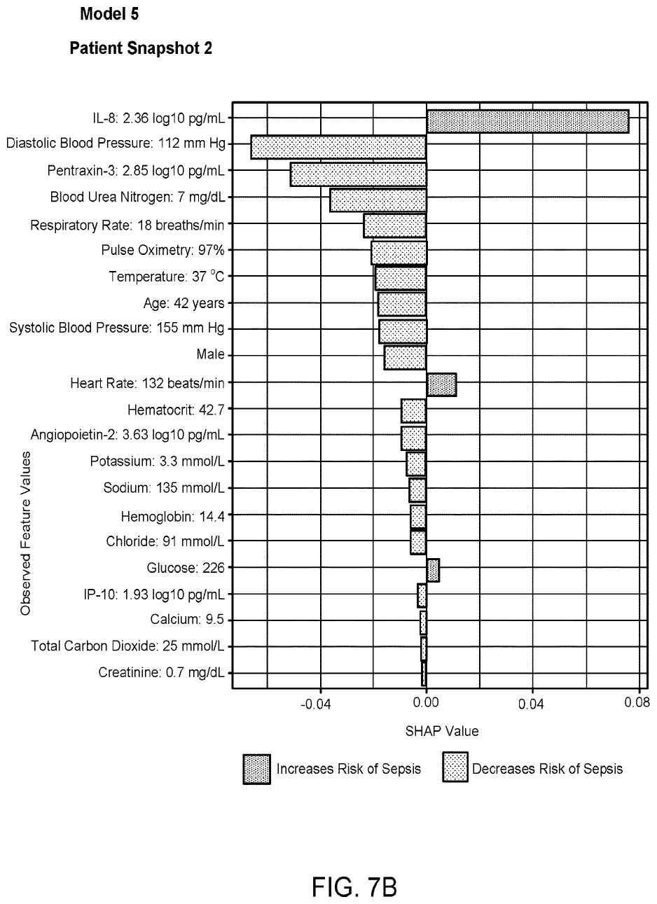

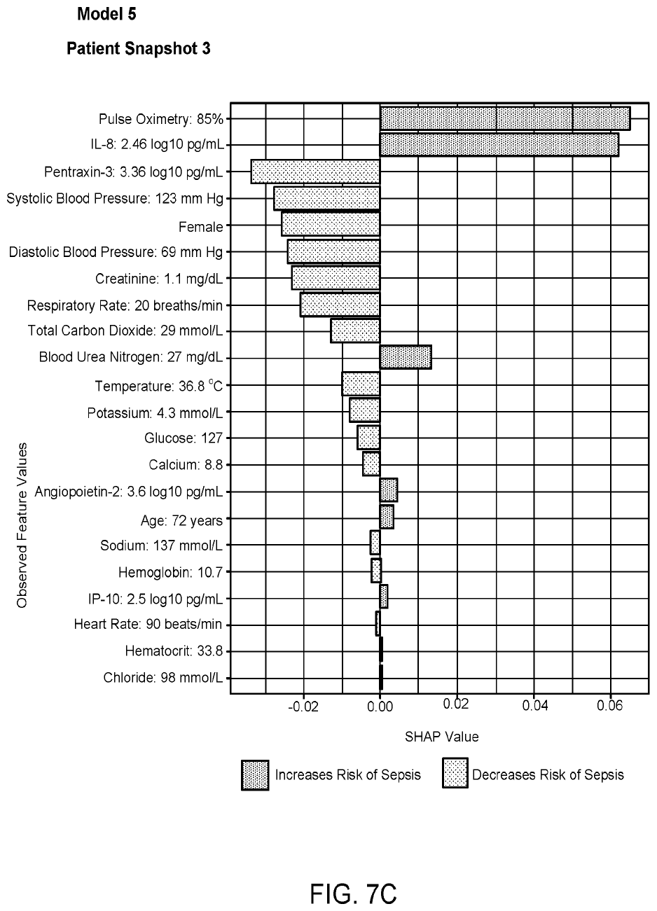

In one embodiment for Model 5, an example of SHAP values for three different patients at risk for sepsis is provided in FIG. 7A (septic within 24 hours), FIG. 7B (not septic within 24 hours), and FIG. 7C (not septic within 24 hours). The input features have a different order of importance among the three patients.

FIG. 7A illustrates a snapshot of the Patient 1 SHAP values for the noted input features. Abnormal blood pressure denoting cardiovascular organ dysfunction gave those features the strongest contribution to this patient's sepsis risk score.

FIG. 7B illustrates a snapshot of the Patient 2 SHAP values for the noted input features. This patient has negative SHAP values for most features, but the most significant individual contribution was their IL-8 value.

FIG. 7C illustrates a snapshot of the Patient 3 SHAP values for the noted input features. Abnormal pulse oximetry and IL-8 have significantly positive SHAP values, while most of their other lab and biomarker values make negative contributions to their sepsis risk score.

Exemplary results for Model 5 diagnostic performance and prognostic performance are as follows:

### Diagnostic Performance

| Risk | | | | Likelihood |
|---|---|---|---|---|
| Group | Septic | Not Septic | Percentage Septic | Ratio |
| Low | 9 | 184 | 4.66% [2.83%, | 0.13 [0.08, |
| | | | 7.26%] | 0.19] |
| Medium | 30 | 141 | 17.54% [13.83%, | 0.58 [0.44, |
| | | | 21.85%] | 0.72] |
| High | 131 | 214 | 37.97% [34.54%, | 1.66 [1.51, |
| | | | 41.51%] | 1.83] |
| Very | 31 | 6 | 83.78% [73.27%, | 14.01 [8.61, |
| High | | | 91.25%] | 27.89] |

### Prognostic Performance

| | | | | Ventilated | Vasopressors |
|---|---|---|---|---|---|
| Risk | In-Hospital | Length | ICU within | within 24 | within 24 |
| Group | Mortality | of Stay | 24 Hours | Hours | Hours |
| Low | 0.52% | 3.81 | 8.29% | 2.07% | 0.00% |
| | [0.05%, | [3.37, | [5.83%, | [0.91%, | [0.00%, |
| | 2.00%] | 4.35] | 11.44%] | 4.10%] | 1.19%] |
| Medium | 3.51% | 5.24 | 16.37% | 1.17% | 2.34% |
| | [1.85%, | [4.75, | [12.77%, | [0.31%, | [1.02%, |
| | 6.08%] | 6.35] | 20.59%] | 3.08%] | 4.62%] |
| High | 8.41% | 7.07 | 35.65% | 7.83% | 10.14% |
| | [6.53%, | [6.36, | [32.27%, | [6.02%, | [8.09%, |
| | 10.65%] | 8.38] | 39.16%] | 10.01%] | 12.55%] |
| Very | 37.84% | 19.08 | 62.16% | 24.32% | 45.95% |
| High | [27.02%, | [11.75, | [50.29%, | [15.24%, | [34.51%, |
| | 49.71%] | NA] | 72.98%] | 35.67%] | 57.73%] |

### VI. Embodiments for Input Features

Particular embodiments of the disclosure provide risk stratification associated with long-term and short-term prognostic clinical outcomes based on analysis of one or more input features of any one or more of Tables 8-13.

In various embodiments, an indicator of risk for sepsis for an individual is provided by one or more input features of any one or more of Tables 8-13. In particular embodiments, an indicator of risk for sepsis for an individual is provided by 1, 2, 3, 4, 5, 6, 7, 8, 9, 10, 11, 12, 13, 14, 15, 16, 17, 18, 19, 20, 21, or 22 input features of Model 1A in Table 8. In particular embodiments, an indicator of risk for sepsis for an individual is provided by 1, 2, 3, 4, 5, 6, 7, 8, 9, 10, 11, 12, 13, 14, 15, 16, 17, 18, 19, 20, 21, 22, or 23 input features of Model 1B in Table 9. In particular embodiments, an indicator of risk for sepsis for an individual is provided by 1, 2, 3, 4, 5, 6, 7, 8, 9, 10, 11, 12, 13, 14, 15, 16, 17, 18, 19, 20, 21, 22, 23, 24, 25, 26, or 27 input features of Model 2 in Table 10. In particular embodiments, an indicator of risk for sepsis for an individual is provided by 1, 2, 3, 4, 5, 6, 7, 8, 9, 10, 11, 12, 13, 14, 15, 16, 17, 18, 19, 20, 21, 22, 23, 24, 25, 26, 27, 28, or 29 input features of Model 3 in Table 11. In particular embodiments, an indicator of risk for sepsis for an individual is provided by 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or 58 input features of Model 4 in Table 12. In particular embodiments, an indicator of risk for sepsis for an individual is provided by 1, 2, 3, 4, 5, 6, 7, 8, 9, 10, 11, 12, 13, 14, 15, 16, 17, 18, 19, 20, 21, or 22 input features of Model 5 in Table 13.

In specific embodiments, at least 2, 3, 4, 5, 6, 7, 8, 9, 10, 11, 12, 13, 14, 15, 16, 17, 18, 19, 20, 21, or 22 input features of Model 1A are utilized as an indicator of risk for sepsis for an individual. In specific embodiments, at least 2, 3, 4, 5, 6, 7, 8, 9, 10, 11, 12, 13, 14, 15, 16, 17, 18, 19, 20, 21, 22, or 23 input features of Model 1B are utilized as an indicator of risk for sepsis for an individual. In specific embodiments, at least 2, 3, 4, 5, 6, 7, 8, 9, 10, 11, 12, 13, 14, 15, 16, 17, 18, 19, 20, 21, 22, 23, 24, 25, 26, or 27 input features of Model 2 are utilized as an indicator of risk for sepsis for an individual. In specific embodiments, at least 2, 3, 4, 5, 6, 7, 8, 9, 10, 11, 12, 13, 14, 15, 16, 17, 18, 19, 20, 21, 22, 23, 24, 25, 26, 27, 28, or 29 input features of Model 3 are utilized as an indicator of risk for sepsis for an individual. In specific embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or 58 input features of Model 4 are utilized as an indicator of risk for sepsis for an individual. In specific embodiments, at least 2, 3, 4, 5, 6, 7, 8, 9, 10, 11, 12, 13, 14, 15, 16, 17, 18, 19, 20, 21, or 22 input features of Model 5 are utilized as an indicator of risk for sepsis for an individual.

In specific embodiments, no more than 2, 3, 4, 5, 6, 7, 8, 9, 10, 11, 12, 13, 14, 15, 16, 17, 18, 19, 20, 21, or 22 input features of Model 1A are utilized as an indicator of risk for sepsis for an individual. In specific embodiments, no more than 2, 3, 4, 5, 6, 7, 8, 9, 10, 11, 12, 13, 14, 15, 16, 17, 18, 19, 20, 21, 22, or 23 input features of Model 1B are utilized as an indicator of risk for sepsis for an individual. In specific embodiments, no more than 2, 3, 4, 5, 6, 7, 8, 9, 10, 11, 12, 13, 14, 15, 16, 17, 18, 19, 20, 21, 22, 23, 24, 25, 26, or 27 input features of Model 2 are utilized as an indicator of risk for sepsis for an individual. In specific embodiments, no more than 2, 3, 4, 5, 6, 7, 8, 9, 10, 11, 12, 13, 14, 15, 16, 17, 18, 19, 20, 21, 22, 23, 24, 25, 26, 27, 28, or 29 input features of Model 3 are utilized as an indicator of risk for sepsis for an individual. In specific embodiments, no more than 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or 58 input features of Model 4 are utilized as an indicator of risk for sepsis for an individual. In specific embodiments, no more than 2, 3, 4, 5, 6, 7, 8, 9, 10, 11, 12, 13, 14, 15, 16, 17, 18, 19, 20, 21, or 22 input features of Model 5 are utilized as an indicator of risk for sepsis for an individual.

In various embodiments, the majority of input features of Model 1A, 1B, 2, 3, 4, or 5 are utilized as an indicator of risk for sepsis for an individual. In some embodiments, at least 50, 55, 60, 65, 70, 75, 80, 85, 90, or 95% of the input features of Model 1A, 1B, 2, 3, 4, or 5 are utilized as an indicator of risk for sepsis for an individual.

In particular embodiments, at least 2, 3, 4, 5, 6, 7, 8, 9, 10, 11, 12, 13, 14, 15, 16, 17, 18, 19, 20, 21, or 22 of the input features of Model 1A are utilized to determine risk for an individual to develop sepsis or to determine that they have sepsis. In specific cases, the order of importance of the input features may be patient-specific.

In particular embodiments, at least 2, 3, 4, 5, 6, 7, 8, 9, 10, 11, 12, 13, 14, 15, 16, 17, 18, 19, 20, 21, 22, or 23 of the input features of Model 1B are utilized to determine risk for an individual to develop sepsis or to determine that they have sepsis. In specific cases, the order of importance of the input features may be patient-specific.

In particular embodiments, at least 2, 3, 4, 5, 6, 7, 8, 9, 10, 11, 12, 13, 14, 15, 16, 17, 18, 19, 20, 21, 22, 23, 24, 25, 26, or 27 of the input features of Model 2 are utilized to determine risk for an individual to develop sepsis or to determine that they have sepsis. In specific cases, the order of importance of the input features may be patient-specific.

In particular embodiments, at least 2, 3, 4, 5, 6, 7, 8, 9, 10, 11, 12, 13, 14, 15, 16, 17, 18, 19, 20, 21, 22, 23, 24, 25, 26, 27, 28, or 29 of the input features of Model 3 are utilized to determine risk for an individual to develop sepsis or to determine that they have sepsis. In specific cases, the order of importance of the input features may be patient-specific.

In particular embodiments, at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, or 58 of the input features of Model 4 are utilized to determine risk for an individual to develop sepsis or to determine that they have sepsis. In specific cases, the order of importance of the input features may be patient-specific.

In particular embodiments, at least 2, 3, 4, 5, 6, 7, 8, 9, 10, 11, 12, 13, 14, 15, 16, 17, 18, 19, 20, 21, or 22 of the input features of Model 5 are utilized to determine risk for an individual to develop sepsis or to determine that they have sepsis. In specific cases, the order of importance of the input features may be patient-specific.

In any model encompassed herein, age of the patient may or may not be the most important input feature for determination of risk of developing sepsis or determination of the presence of sepsis. In any model encompassed herein, albumin level of the patient may or may not be the most important input feature for determination of risk of developing sepsis or determination of the presence of sepsis. In any model encompassed herein, angiopoietin-1 level of the patient may or may not be the most important input feature for determination of risk of developing sepsis or determination of the presence of sepsis. In any model encompassed herein, angiopoietin-2 level of the patient may or may not be the most important input feature for determination of risk of developing sepsis or determination of the presence of sepsis. In any model encompassed herein, bilirubin level of the patient may or may not be the most important input feature for determination of risk of developing sepsis or determination of the presence of sepsis. In any model encompassed herein, biological sex of the patient may or may not be the most important input feature for determination of risk of developing sepsis or determination of the presence of sepsis, such as being female. In any model encompassed herein, blood urea nitrogen (BUN) level of the patient may or may not be the most important input feature for determination of risk of developing sepsis or determination of the presence of sepsis. In any model encompassed herein, chloride level of the patient may or may not be the most important input feature for determination of risk of developing sepsis or determination of the presence of sepsis. In any model encompassed herein, CO₂ level of the patient may or may not be the most important input feature for determination of risk of developing sepsis or determination of the presence of sepsis. In any model encompassed herein, C-reactive protein level of the patient may or may not be the most important input feature for determination of risk of developing sepsis or determination of the presence of sepsis. In any model encompassed herein, creatinine level of the patient may or may not be the most important input feature for determination of risk of developing sepsis or determination of the presence of sepsis. In any model encompassed herein, diastolic blood pressure level of the patient may or may not be the most important input feature for determination of risk of developing sepsis or determination of the presence of sepsis. In any model encompassed herein, E-selectin level of the patient may or may not be the most important input feature for determination of risk of developing sepsis or determination of the presence of sepsis. In any model encompassed herein, FLT-3 Ligand level of the patient may or may not be the most important input feature for determination of risk of developing sepsis or determination of the presence of sepsis. In any model encompassed herein, fractalkine level of the patient may or may not be the most important input feature for determination of risk of developing sepsis or determination of the presence of sepsis. In any model encompassed herein, GCSF level of the patient may or may not be the most important input feature for determination of risk of developing sepsis or determination of the presence of sepsis. In any model encompassed herein, GMCSF level of the patient may or may not be the most important input feature for determination of risk of developing sepsis or determination of the presence of sepsis. In any model encompassed herein, granzyme B level of the patient may or may not be the most important input feature for determination of risk of developing sepsis or determination of the presence of sepsis. In any model encompassed herein, glucose level of the patient may or may not be the most important input feature for determination of risk of developing sepsis or determination of the presence of sepsis. In any model encompassed herein, heart rate of the patient may or may not be the most important input feature for determination of risk of developing sepsis or determination of the presence of sepsis. In any model encompassed herein, hematocrit level of the patient may or may not be the most important input feature for determination of risk of developing sepsis or determination of the presence of sepsis. In any model encompassed herein, hemoglobin level of the patient may or may not be the most important input feature for determination of risk of developing sepsis or determination of the presence of sepsis. In any model encompassed herein, IFN-alpha level of the patient may or may not be the most important input feature for determination of risk of developing sepsis or determination of the presence of sepsis. In any model encompassed herein, IFN-gamma level of the patient may or may not be the most important input feature for determination of risk of developing sepsis or determination of the presence of sepsis. In any model encompassed herein, IL-10 level of the patient may or may not be the most important input feature for determination of risk of developing sepsis or determination of the presence of sepsis. In any model encompassed herein, IL-15 level of the patient may or may not be the most important input feature for determination of risk of developing sepsis or determination of the presence of sepsis. In any model encompassed herein, IL1-beta level of the patient may or may not be the most important input feature for determination of risk of developing sepsis or determination of the presence of sepsis. In any model encompassed herein, IL1-RA level of the patient may or may not be the most important input feature for determination of risk of developing sepsis or determination of the presence of sepsis. In any model encompassed herein, IL-2 level of the patient may or may not be the most important input feature for determination of risk of developing sepsis or determination of the presence of sepsis. In any model encompassed herein, IL-4 level of the patient may or may not be the most important input feature for determination of risk of developing sepsis or determination of the presence of sepsis. In any model encompassed herein, IL-6 level of the patient may or may not be the most important input feature for determination of risk of developing sepsis or determination of the presence of sepsis. In any model encompassed herein, IL-7 level of the patient may or may not be the most important input feature for determination of risk of developing sepsis or determination of the presence of sepsis. In any model encompassed herein, IL-8 level of the patient may or may not be the most important input feature for determination of risk of developing sepsis or determination of the presence of sepsis. In any model encompassed herein, IL-10 level of the patient may or may not be the most important input feature for determination of risk of developing sepsis or determination of the presence of sepsis. In any model encompassed herein, IP-10 level of the patient may or may not be the most important input feature for determination of risk of developing sepsis or determination of the presence of sepsis. In any model encompassed herein, lactate level of the patient may or may not be the most important input feature for determination of risk of developing sepsis or determination of the presence of sepsis. In any model encompassed herein, leptin level of the patient may or may not be the most important input feature for determination of risk of developing sepsis or determination of the presence of sepsis. In any model encompassed herein, lymphocyte level of the patient may or may not be the most important input feature for determination of risk of developing sepsis or determination of the presence of sepsis. In any model encompassed herein, MCP1 level of the patient may or may not be the most important input feature for determination of risk of developing sepsis or determination of the presence of sepsis. In any model encompassed herein, MIP1-alpha level of the patient may or may not be the most important input feature for determination of risk of developing sepsis or determination of the presence of sepsis. In any model encompassed herein, MIP1-beta level of the patient may or may not be the most important input feature for determination of risk of developing sepsis or determination of the presence of sepsis. In any model encompassed herein, MIP3-alpha level of the patient may or may not be the most important input feature for determination of risk of developing sepsis or determination of the presence of sepsis. In any model encompassed herein, neutrophil level of the patient may or may not be the most important input feature for determination of risk of developing sepsis or determination of the presence of sepsis. In any model encompassed herein, NGAL level of the patient may or may not be the most important input feature for determination of risk of developing sepsis or determination of the presence of sepsis. In any model encompassed herein, platelet level of the patient may or may not be the most important input feature for determination of risk of developing sepsis or determination of the presence of sepsis. In any model encompassed herein, programmed death-ligand 1 (PD-L1) level of the patient may or may not be the most important input feature for determination of risk of developing sepsis or determination of the presence of sepsis. In any model encompassed herein, pentraxin-3 level of the patient may or may not be the most important input feature for determination of risk of developing sepsis or determination of the presence of sepsis. In any model encompassed herein, potassium level of the patient may or may not be the most important input feature for determination of risk of developing sepsis or determination of the presence of sepsis. In any model encompassed herein, platelet level of the patient may or may not be the most important input feature for determination of risk of developing sepsis or determination of the presence of sepsis. In any model encompassed herein, procalcitonin level of the patient may or may not be the most important input feature for determination of risk of developing sepsis or determination of the presence of sepsis. In any model encompassed herein, pulse oximetry of the patient may or may not be the most important input feature for determination of risk of developing sepsis or determination of the presence of sepsis. In any model encompassed herein, respiratory rate of the patient may or may not be the most important input feature for determination of risk of developing sepsis or determination of the presence of sepsis. In any model encompassed herein, sodium level of the patient may or may not be the most important input feature for determination of risk of developing sepsis or determination of the presence of sepsis. In any model encompassed herein, systolic blood pressure of the patient may or may not be the most important input feature for determination of risk of developing sepsis or determination of the presence of sepsis. In any model encompassed herein, temperature of the patient may or may not be the most important input feature for determination of risk of developing sepsis or determination of the presence of sepsis. In any model encompassed herein, TGF-alpha level of the patient may or may not be the most important input feature for determination of risk of developing sepsis or determination of the presence of sepsis. In any model encompassed herein, thromodulin level of the patient may or may not be the most important input feature for determination of risk of developing sepsis or determination of the presence of sepsis. In any model encompassed herein, Tissue Factor level of the patient may or may not be the most important input feature for determination of risk of developing sepsis or determination of the presence of sepsis. In any model encompassed herein, TNF-alpha level of the patient may or may not be the most important input feature for determination of risk of developing sepsis or determination of the presence of sepsis. In any model encompassed herein, TRAIL level of the patient may or may not be the most important input feature for determination of risk of developing sepsis or determination of the presence of sepsis. In any model encompassed herein, triggering receptor expressed on myeloid cells 1 (TREM-1) level of the patient may or may not be the most important input feature for determination of risk of developing sepsis or determination of the presence of sepsis. In any model encompassed herein, VCAM-1 level of the patient may or may not be the most important input feature for determination of risk of developing sepsis or determination of the presence of sepsis. In any model encompassed herein, VEG-F level of the patient may or may not be the most important input feature for determination of risk of developing sepsis or determination of the presence of sepsis. In any model encompassed herein, white blood cell count of the patient may or may not be the most important input feature for determination of risk of developing sepsis or determination of the presence of sepsis.

In various embodiments, methods of the disclosure generate an indicator of risk for sepsis, such as with a predicted score that indicates a probability of a patient having or developing sepsis, e.g., within a predetermined time-span based on at least one input features listed for any one or more of the models in Tables 8-13. In some embodiments, a particular input feature has greater importance in the predicted score than one or more other input features.

In specific embodiments with respect to Model 1A, age has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, albumin level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, bilirubin level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, BUN level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, chloride level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, CO₂ has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, C-Reactive protein level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, Creatinine level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, diastolic blood pressure has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, heart rate has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, lactate level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, lymphocyte level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, neutrophil level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, platelet level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, potassium level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, procalcitonin level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, pulse oximetry has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, respiratory rate has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, sodium level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, systolic blood pressure has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, temperature has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 1A, white blood cell count has greater importance in the predicted score than 1, 2, 3, 4, 5, 6, 7, 8, 9, 10, 11, 12, 13, 14, 15, 16, 17, 18, 19, 20, or 21 other input features.

In specific embodiments with respect to Model 1B, age has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, albumin level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, bilirubin level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, BUN level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, chloride level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, CO₂ has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, C-Reactive protein level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, Creatinine level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, diastolic blood pressure has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, heart rate has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, IL-6 level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, lactate level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, lymphocyte level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, neutrophil level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, platelet level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, potassium level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, procalcitonin level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, pulse oximetry has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, respiratory rate has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, sodium level has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, systolic blood pressure has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, temperature has greater importance in the predicted score than 1, 2, 3, 4, 5, 6, 7, 8, 9, 10, 11, 12, 13, 14, 15, 16, 17, 18, 19, 20, 21, or 22 other input features. In specific embodiments with respect to Model 1B, white blood cell count has greater importance in the predicted score than 1, 2, 3, 4, 5, 6, 7, 8, 9, 10, 11, 12, 13, 14, 15, 16, 17, 18, 19, 20, 21, or 22 other input features.

In specific embodiments with respect to Model 2, age has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, albumin level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, angiopoietin-2 level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, bilirubin level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, BUN level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, chloride level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, CO₂ level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, C-reactive protein level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, creatinine level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, diastolic blood pressure has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, heart rate has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, IL-6 level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, IL-8 level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, IP-10 level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, lactate level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, lymphocyte level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, neutrophil level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, pentraxin-3 level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, platelet level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, potassium level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, procalcitonin level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, pulse oximetry has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, respiratory rate has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, sodium level has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, systolic blood pressure has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, temperature has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features. In specific embodiments with respect to Model 2, white blood cell count has greater importance in the predicted score than 1, 2, 3, 4, 5, 6, 7, 8, 9, 10, 11, 12, 13, 14, 15, 16, 17, 18, 19, 20, 21, 22, 23, 24, 25, or 26 other input features.

In specific embodiments with respect to Model 3, age has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, albumin level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, angiopoietin-2 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, bilirubin level has greater importance in the predicted score than 1, 2, 3, 4, 5, 6, 7, 8, 9, 10, It, 12, 13, 14, 15, 16, 17, 18, 19, 20, 21, 22, 23, 24, 25, 26, 27, or 28 other input features. In specific embodiments with respect to Model 3, BUN level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, chloride level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, CO₂ has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, C-Reactive protein level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, creatinine level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, diastolic blood pressure has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, heart rate has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, IL-6 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, IL-8 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, IP-10 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, lactate level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, lymphocyte level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, neutrophil level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, PD-L1 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, pentraxin-3 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, platelet level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, potassium level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, procalcitonin level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, pulse oximetry has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, respiratory rate has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, sodium level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, systolic blood pressure has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, temperature has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, TREM-1 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features. In specific embodiments with respect to Model 3, white blood cell count has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, or 28 other input features.

In specific embodiments with respect to Model 4, age has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, albumin level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, angiopoietin-1 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, angiopoietin-2 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, bilirubin level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, BUN level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, chloride level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, CO₂ level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, C-Reactive protein level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, creatinine level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, diastolic blood pressure has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, E-Selectin level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, FLT3-Ligand level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, fractalkine level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, GCSF level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, GMCSF level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, granzyme-B level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, heart rate has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, IFN-alpha level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, IFN-gamma level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, IL-10 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, IL-15 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, IL1-beta level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, IL1-RA level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, O-2 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, IL-4 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, IL-6 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, IL-7 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, IL-8 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, IP-10 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, lactate level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, leptin level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, lymphocyte level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, MCP1 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, MIP1-alpha level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, MIP1-beta level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, MIP3-alpha level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, neutrophil level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, NGAL level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, PD-L1 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, pentraxin-3 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, platelet level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, potassium level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, procalcitonin level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, pulse oximetry has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, respiratory rate has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, sodium level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, systolic blood pressure has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, temperature has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, TGF-alpha level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, thromodulin level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, Tissue Factor level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, TNF-alpha level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, TRAIL level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, TREM-1 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, VCAM-1 level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, VEGF level has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features. In specific embodiments with respect to Model 4, white blood cell count has greater importance in the predicted score than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, or 57 other input features.

In specific embodiments with respect to Model 5, age has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, angiopoietin-2 level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, BUN level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, calcium level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, chloride level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, CO₂ has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, Creatinine level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, diastolic blood pressure has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, biological sex has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, glucose level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, heart rate has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, hematocrit has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, hemoglobin has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, IL-8 level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, IP-10 level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, pentraxin-3 level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, potassium level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, pulse oximetry has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, respiratory rate has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, sodium level has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, systolic blood pressure has greater importance in the predicted score than 1, 2, 3, 4, 5, 6, 7, 8, 9, 10, 11, 12, 13, 14, 15, 16, 17, 18, 19, 20, or 21 other input features. In specific embodiments with respect to Model 5, temperature has greater importance in the predicted score than 1, 2, 3, 4, 5, 6, 7, 8, 9, 10, 11, 12, 13, 14, 15, 16, 17, 18, 19, 20, or 21 other input features.

### VII. Embodiments of Treatment Methods

In some embodiments, an individual having been determined to have sepsis or be at risk for sepsis (including at risk within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 341, 32, 33, 34, 35, 36, 37, 38, 39, 40, 41, 42, 43, 44, 45, 46, 47, 48, or more hours) may be provided with a therapeutically effective amount of one or more therapies. In specific embodiments, the therapy comprises one or more antibiotics that may be administered orally (by pill, liquid, or capsule), by injection, drops, and/or topical (cream or ointment), and/or intravenously, for example. The treatment may be on the order of 1-24, 1-12, 1-6, 6-24, 6-18, 6-12, 12-24, 12-18, or 18-24 hours; 1-7, 1-6, 1-5, 1-4, 1-3, 1-2, 2-7, 2-6, 2-5, 2-4, 2-3, 3-7, 3-6, 3-5, 3-4, 4-7, 4-6, 4-5, 5-7, 5-6, or 6-7 days; or 1-4, 1-3, 1-2, 2-4, 2-3, or 3-4 or more weeks, for example, depending on the type of infection and how it reacts to the antibiotics. In some embodiments a first treatment is ineffective or less effective than desired, and a second treatment may be needed, such as a different type of antibiotic.

In particular embodiments when an individual is in need of an antibiotic, one or more of the following may be administered: (1) ceftriaxone (e.g., 1 to 2 g IV or IM every 12 to 24 hours for adults; 50 to 75 mg/kg/day IV or IM divided every 12 to 24 hours for infants, children, and adolescents; and 50 mg/kg/dose IV or IM every 24 hours for neonates and pre-term infants); (2) piperacillin-tazobactam (e.g., 4.5 g (4 g piperacillin and 0.5 g tazobactam) IV every 6 hours for adults; 80 mg/kg/dose piperacillin component (90 mg/kg/dose piperacillin; tazobactam) IV every 6 hours for infants, children, and adolescents; and 80 or 100 mg/kg/dose piperacillin component (90 mg/kg/dose piperacillin; tazobactam) IV every 6 hours for neonates); (3) cefepime (e.g., 2 g IV every 8 hours for adults; 50 mg/kg/dose (Max: 2 g/dose) IV every 8 hours for infants, children, and adolescents; and 30 or 50 mg/kg/dose IV every 12 hours) for neonates); (4) ceftazidime (e.g., 2 g IV every 8 hours for adults and adolescents; 90 to 150 mg/kg/day (Max: 6 g/day) IV divided every 8 hours for infants and children; 50 mg/kg/dose IV every 8 hours or 12 hrs); (5) vancomycin (e.g. 20 to 35 mg/kg/dose (Max: 3,000 mg/dose) IV loading dose, followed by 15 to 20 mg/kg/dose IV every 8 to 12 hours for adults; 60 to 70 mg/kg/day IV divided every 6 to 8 hours for children and adolescents; 60 to 80 mg/kg/day IV divided every 6 hours for infants); and/or (6) ciprofloxacin (e.g., 600 mg IV every 12 hours for adults).

Embodiments of the disclosure include methods of treatment for sepsis in individuals having sepsis or suspected of having sepsis or at risk for sepsis within 1-48 hours, including in individuals for which input features for the individual using a machine learning model generated a predicted score that indicated that the individual had sepsis or that the individual would develop sepsis, such as within a predetermined time-span (e.g., within 1-48 hours).

Embodiments of the disclosure include methods wherein generating a diagnosis output based on a predicted score from any method encompassed herein classifies the patient as having sepsis, or being at risk for developing sepsis within a predetermined time-span, the method further comprising administering to the individual a therapeutically effective amount of a treatment for sepsis. In specific embodiments, the treatment comprises at least one of an antibiotic or an intravenous fluid based on the generated diagnosis output.

Embodiments of the disclosure include methods of treating an individual for sepsis having a score based on multiple input features that indicates a probability of the patient having or developing sepsis based on any method encompassed herein, said treating comprising administering to the individual an effective amount of one or more sepsis therapies.

Embodiments of the disclosure include methods comprising measuring multiple input features in Table 8, Table 9, Table 10, Table 11, Table 12, and/or Table 13 of an individual and/or from a sample from an individual, optionally wherein measuring of at least a first input feature and at least a second input feature is successive, and the measuring of the second input feature is predicated upon the outcome of the measuring of the first input feature.

Embodiments of the disclosure include methods comprising measuring 1, 2, 3, 4, 5, 6, 7, 8, 9, 10, 11, 12, 13, 14, 15, 16, 17, 18, 19, 20, 21, or more input features in any one or more of Tables 8, 9, 10, 11, 12, or 13 of an individual. In specific embodiments, the individual has one or more symptoms of sepsis. In some embodiments, the individual is suspected of having sepsis, suspected of developing sepsis, or is at risk for developing sepsis. In some embodiments, the individual is suspected of developing sepsis within 24 hours of the measuring in a sample obtained from the individual.

Embodiments of the disclosure include methods of treating an individual for sepsis, said individual having one or more particular values of any input features of any one or more of Tables 8, 9, 10, 11, 12, or 13, said input features indicative of sepsis.

Embodiments of the disclosure include methods of treating an individual having one or more particular values of any one or more input features of any one or more of Tables 8, 9, 10, 11, 12, or 13, said treating comprising administering a therapeutically effective amount of one or more antibiotics.

### VIII. Computer Implemented System

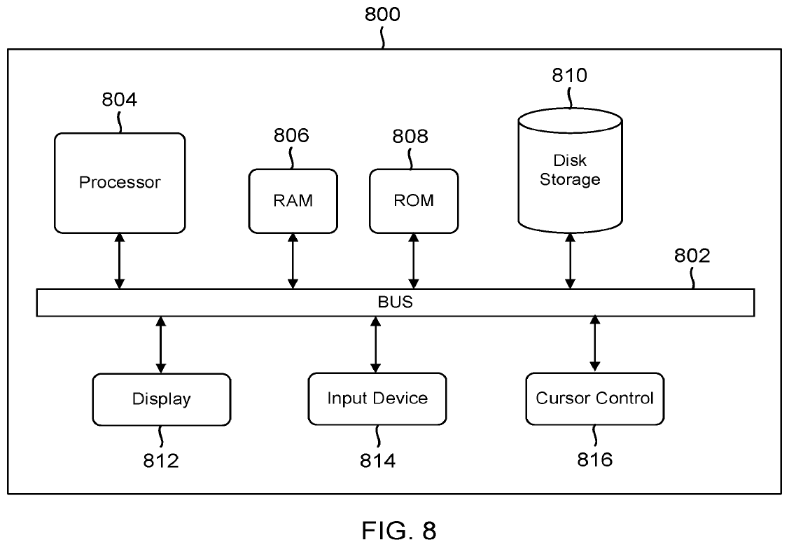

FIG. 8 is a block diagram of a computer system, in accordance with various embodiments. Computer system 800 may be an example of one implementation for various methods such as method 200 described above in FIG. 2.

In one or more examples, computer system 800 can include a bus 802 or other communication mechanism for communicating information, and a processor 804 coupled with bus 802 for processing information. In various embodiments, computer system 800 can also include a memory, which can be a random-access memory (RAM) 806 or other dynamic storage device, coupled to bus 802 for determining instructions to be executed by processor 804. Memory also can be used for storing temporary variables or other intermediate information during execution of instructions to be executed by processor 804. In various embodiments, computer system 800 can further include a read only memory (ROM) 808 or other static storage device coupled to bus 802 for storing static information and instructions for processor 804. A storage device 810, such as a magnetic disk or optical disk, can be provided and coupled to bus 802 for storing information and instructions.

In various embodiments, computer system 800 can be coupled via bus 802 to a display 812, such as a cathode ray tube (CRT), liquid crystal display (LCD), or light emitting diode (LED) for displaying information to a computer user. An input device 814, including alphanumeric and other keys, can be coupled to bus 802 for communicating information and command selections to processor 804. Another type of user input device is a cursor control 816, such as a mouse, a joystick, a trackball, a gesture input device, a gaze-based input device, or cursor direction keys for communicating direction information and command selections to processor 804 and for controlling cursor movement on display 812. This input device 814 typically has two degrees of freedom in two axes, a first axis (e.g., x) and a second axis (e.g., y), that allows the device to specify positions in a plane. However, it should be understood that input devices 814 allowing for three-dimensional (e.g., x, y, and z) cursor movement are also contemplated herein.

Consistent with certain implementations of the present teachings, results can be provided by computer system 800 in response to processor 804 executing one or more sequences of one or more instructions contained in RAM 806. Such instructions can be read into RAM 806 from another computer-readable medium or computer-readable storage medium, such as storage device 810. Execution of the sequences of instructions contained in RAM 806 can cause processor 804 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, storage device, data storage device, etc.) or “computer-readable storage medium” as used herein refers to any media that participates in providing instructions to processor 804 for execution. Such a medium can take many forms, including but not limited to, non-volatile media, volatile media, and transmission media. Examples of non-volatile media can include, but are not limited to, optical, solid state, magnetic disks, such as storage device 810. Examples of volatile media can include, but are not limited to, dynamic memory, such as RAM 806. Examples of transmission media can include, but are not limited to, coaxial cables, copper wire, and fiber optics, including the wires that comprise bus 802.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 804 of computer system 800 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, optical communications connections, etc.

It should be appreciated that the methodologies described herein, flow charts, diagrams, and accompanying disclosure can be implemented using computer system 800 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 800, whereby processor 804 would execute the analyses and determinations provided by these engines, subject to instructions provided by any one of, or a combination of, the memory components RAM 806, ROM, 808, or storage device 810 and user input provided via input device 814.

### IX. Additional Considerations

Any headers and/or subheaders between sections and subsections of this document are included solely for the purpose of improving readability and do not imply that features cannot be combined across sections and subsection. Accordingly, sections and subsections do not describe separate embodiments.

While the present teachings are described in conjunction with various embodiments, it is not intended that the present teachings be limited to such embodiments. On the contrary, the present teachings encompass various alternatives, modifications, and equivalents, as will be appreciated by those of skill in the art. The present description provides preferred exemplary embodiments, and is not intended to limit the scope, applicability, or configuration of the disclosure. Rather, the present description of the preferred exemplary embodiments will provide those skilled in the art with an enabling description for implementing various embodiments.

It is understood that various changes may be made in the function and arrangement of elements without departing from the spirit and scope as set forth in the appended claims. Thus, such modifications and variations are considered to be within the scope set forth in the appended claims. Further, the terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

Specific details are given in the present description to provide an understanding of the embodiments. However, it is understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

### Embodiments

1. A method of generating an indicator of risk for sepsis, the method comprising: receiving at least one input feature corresponding to a patient;
analyzing the at least one input feature using a machine learning model to generate a predicted score that indicates a probability of the patient having or developing sepsis within a predetermined time-span based on the at least one input feature; wherein the at least one input feature is associated with sepsis.
2. The method of embodiment 1, wherein the at least one input feature comprises a clinical parameter.
3. The method of embodiment 1 or 2, wherein the at least one input feature comprises a biomarker.
4. The method of any one of embodiments 1-3, further comprising generating a diagnosis output based on the predicted score.
5. The method of any one of embodiments 1-4, wherein the clinical parameter and/or the biomarker is/are selected from a group of input features listed in Table 8.
6. The method of any one of embodiments 1-4, wherein the clinical parameter and/or the biomarker is/are selected from a group of input features listed in Table 9.
7. The method of any one of embodiments 1-4, wherein the clinical parameter and/or the biomarker is/are selected from a group of input features listed in Table 10.
8. The method of any one of embodiments 1-4, wherein the clinical parameter and/or the biomarker is/are selected from a group of input features listed in Table 11.
9. The method of any one of embodiments 1-4, wherein the clinical parameter and/or the biomarker is/are selected from a group of input features listed in Table 12.
10. The method of any one of embodiments 1-4, wherein the clinical parameter and/or the biomarker is/are selected from a group of input features listed in Table 13.
11. The method of any one of embodiments 5-10, wherein the group of input features listed in and one of Tables 1-6 are listed with respect to relative significance to the predicted score.
12. The method of any one of embodiments 1-11, wherein the relative significance to the predicted score is determined based on feature ranking, wherein the feature ranking is determined by ranking input features based on average SHAP values, wherein a high average SHAP value represent a high relative significance to the predicted score and a low SHAP value representing a low relative significance to the predicted score.
13. The method of embodiment 12, wherein an average SHAP value for an input feature in the group of input features listed in any one of Tables 8-13, is computed by:
   determining absolute magnitudes of all SHAP values for a given population for the input feature;
   summing the absolute magnitudes of all SHAP values to obtain a total summed magnitude of the input feature; and
   dividing the total summed magnitude by a population number of the given population for the input feature.
14. The method of any one of embodiments 1-13, wherein the at least one input feature comprises Procalcitonin, wherein Procalcitonin concentration values are divided into two groups, wherein a first group of Procalcitonin concentration values has a lower relative significance than a second group of Procalcitonin concentration values.
15. The method of embodiment 14, wherein a dividing threshold value between the first group and second group of Procalcitonin concentration values is 2.5 log 10 pm/mL.
16. The method of embodiment 15, wherein the relative significance of Procalcitonin to the predicted score in each of the first group and the second group is a median SHAP value for Procalcitonin concentration values within the respective group.
17. The method of any one of embodiments 1-13, wherein the at least one input feature comprises Procalcitonin, wherein Procalcitonin concentration values are divided into at least three groups, wherein a first group of Procalcitonin concentration values has a lower relative significance than a second group of Procalcitonin concentration values, and wherein a third group of Procalcitonin concentration values has a higher relative significance than both the first group and second group of Procalcitonin concentration values.
18. The method of embodiment 17, wherein a first dividing threshold value between the first and second groups of Procalcitonin concentration values is 1.7 log 10 μg/mL and wherein a second dividing threshold value between the second and third groups of Procalcitonin concentration values is 3.4 log 10 μg/mL.
19. The method of embodiment 18, wherein the relative significance of Procalcitonin to the predicted score in each of the first, second, and third groups is a median SHAP value for Procalcitonin concentration values within the respective group.
20. The method of any one of embodiments 1-19, wherein the group of input features listed Table 1 comprises Glasgow coma scale, FiO2, Procalcitonin, systolic blood pressure, and platelet count.
21. The method of embodiment 20, wherein Glasgow coma scale has a higher relative significance than FiO2, which has a higher relative significance than Procalcitonin, which has a higher relative significance than systolic blood pressure, which has a higher relative significance than the platelet count.
22. The method of any one of embodiments 1-19, wherein the group of input features listed Table 1 comprises Glasgow coma scale, FiO₂, Procalcitonin, systolic blood pressure, platelet count, Bilirubin, blood urea nitrogen, respiratory rate, diastolic blood pressure, and Albumin.
23. The method of any one of embodiments 1-19, wherein the group of input features listed Table 1 comprises Glasgow coma scale, FiO2, Procalcitonin, systolic blood pressure, platelet count, Bilirubin, blood urea nitrogen, respiratory rate, diastolic blood pressure, Albumin, Creatinine, age, blood oxygen saturation, Lactate, and C-reactive protein.
24. The method of any one of embodiments 1-19, wherein the group of input features listed Table 1 comprises Glasgow coma scale, FiO₂, Procalcitonin, systolic blood pressure, platelet count, Bilirubin, blood urea nitrogen, respiratory rate, diastolic blood pressure, Albumin, Creatinine, age, blood oxygen saturation, Lactate, C-reactive protein, potassium, neutrophil count, temperature, total carbon dioxide, white blood cell count, lymphocyte count, sodium, heart rate, and chloride.
25. The method of any one of embodiments 1-19, wherein the group of input features comprises Procalcitonin and C-Reactive Protein.
26. The method of embodiment 25, wherein either or both of Procalcitonin and C-Reactive Protein is replaced with one or more other biomarkers listed in Table 2 in accordance with correlation values associated with Table 3.
27. The method of embodiment 26, wherein the one or more other biomarkers replacing either or both of Procalcitonin and C-Reactive Protein has a correlation value smaller than XX.
28. The method of any one of embodiments 1-27, further comprising:
determining that the predicted score falls within one of four categories; and
generating, based on the category in which the predicted score falls, a diagnosis output that includes that the patient has a low, medium, high, or very high probability of having or developing sepsis within the predetermined time-span.
29. The method of embodiment 28, wherein the predicted score ranges between 0 and 1.
30. The method of embodiments 28 or 29, wherein the four categories comprise a low category, a medium category, a high category, and a very high category, and
wherein the low category is designated as having the predicted score below a first threshold, the medium category is designated as having the predicted score between the first threshold and a second threshold, the high category is designated as having the predicted score between the second threshold and a third threshold, and the very high category is designated as having the predicted score above the third threshold.
31. The method of embodiment 30, wherein the predicted score is below the first threshold of 0.122, and the diagnosis output indicates that the patient has a low probability of having or developing sepsis within the predetermined time-span.
32. The method of embodiment 30, wherein the predicted score is at or above the first threshold of 0.122 and below the second threshold of 0.306, and the diagnosis output indicates that the patient has a medium probability of having or developing sepsis within the predetermined time-span.
33. The method of embodiment 30, wherein the predicted score is at or above the second threshold of 0.306 and below the third threshold of 0.872, and the diagnosis output indicates that the patient has a high probability of having or developing sepsis within the predetermined time-span.
34. The method of embodiment 30, wherein the predicted score is at or above the third threshold of 0.872, and the diagnosis output indicates that the patient has a very high probability of having or developing sepsis within the predetermined time-span.
35. The method of any one of embodiments 1-34, further comprising:
calibrating the predicted score using a calibration model to generate a calibrated score; and
generating a risk category as a diagnosis output in view of the calibrated score.
36. The method of embodiment 35, wherein generating the diagnosis output comprises:
determining that the calibrated score falls within one of four risk categories; and
generating, based on the category in which the calibrated score falls, the risk category that includes that the patient has a low, medium, high, or very high probability of having or developing sepsis within the predetermined time-span.
37. The method of embodiment 36, wherein the calibrated score ranges between 0 and 1.
38. The method of any one of embodiments 35-37, wherein the risk category can be selected from a group consisting of a low category, a medium category, a high category, and a very high category, wherein the low category is designated as having the calibrated score below a first threshold, the medium category is designated as having the calibrated score between the first threshold and a second threshold, the high category is designated as having the calibrated score between the second threshold and a third threshold, and the very high category is designated as having the calibrated score above the third threshold.
39. The method of embodiment 38, wherein the calibrated score is below the first threshold of 0.122, and the risk category indicates that the patient has a low probability of having or developing sepsis within the predetermined time-span.
40. The method of embodiment 38, wherein the calibrated score is above the first threshold of 0.122 and below the second threshold of 0.306, and the risk category indicates that the patient has a medium probability of having or developing sepsis within the predetermined time-span.
41. The method of embodiment 38, wherein the calibrated score is above the second threshold of 0.306 and below the third threshold of 0.872, and the risk category indicates that the patient has a high probability of having or developing sepsis within the predetermined time-span.
42. The method of embodiment 38, wherein the calibrated score is above the third threshold of 0.872, and the risk category indicates that the patient has a very high probability of having or developing sepsis within the predetermined time-span.
43. The method of any one of embodiments 1-42, wherein the predetermined time-span is no more than, or more than, 12 hours, 18 hours, 24 hours, 36 hours, 48 hours, or 72 hours.
44. The method of any one of embodiments 1-43, further comprising:
training the at least one machine learning model using training data, wherein the training data comprises a plurality of biomarkers and health profiles for a plurality of patients and a plurality of subject diagnoses for the plurality of patients.
45. The method of any one of embodiments 4-44, wherein the diagnosis output is an automatically derived label based on the predicted score generated by the at least one machine learning model.
46. The method of any one of embodiments 4-45, wherein the diagnosis output is adjudicated by a physician in view of the predicted score generated by the at least one machine learning model.
47. The method of any one of embodiments 4-46, wherein the analysis is performed without a value for one clinical parameter of the plurality of clinical parameters and/or a value for one biomarker of the one or more biomarkers to generate the predicted score, the method further comprising: generating the diagnosis output based on the predicted score.
48. The method of any one of embodiments 1-47, further comprising:
identifying and imputing a value for one input feature of the input features that is missing a value associated with the one input feature.
49. The method of any one of embodiments 1-47, wherein one input feature of the input features has a missing value, the method further comprising:
using a trigger logic decision making module to determine whether to perform the analysis without the missing value for the one input feature to generate the predicted score.
50. The method of any one of embodiments 4-47, wherein one input feature of the input features has a missing value, the method further comprising:
imputing the missing value for the one input feature to generate an imputed value for the missing input feature;
analyzing the data that include the imputed value to generate the predicted score that indicates a probability of the patient having or developing sepsis within a predetermined time-span; and
generating the diagnosis output based on the predicted score.
51. The method of any one of embodiments 48-50, wherein the imputing of the missing value is performed via a pre-trained template that is trained using training data, the training data comprising input features that include a plurality of clinical parameters and a plurality of biomarkers for a plurality of patients, and a plurality of subject diagnoses for the plurality of patients.
52. The method of any one of embodiments 48-51, wherein the one input feature that has the missing value is designated as a feature whose value is measured or obtained outside of a prescribed time window for data acceptable for use in generating the predicted score.
53. The method of embodiment 53, wherein the prescribed time window is a same time period for a first set of input features and is different for at least two input features of a second set of input features.
54. The method of any one of embodiments 1-53, wherein the input feature comprises a clinical parameter, having a numerical value measured within a prescribed time window.
55. The method of any one of embodiments 1-54, wherein the input feature comprises a biomarker having a measurement value associated with the biomarker acquired within a prescribed time window.
56. The method of embodiment 1-55, wherein the input feature comprises a clinical parameter, the clinical parameter comprising at least one of demographic measurements, patient assessments, vital signs, hematology laboratory values, and chemistry laboratory values.
57. The method of any one of embodiments 48-50, further comprising: identifying and imputing a biomarker from the plurality of biomarkers that is missing a value associated with the biomarker.
58. The method of any one of embodiments 48-57, wherein the one input feature that is missing a value associated with the one input feature, comprises a biomarker from the plurality of biomarkers and/or a clinical parameter from the plurality of clinical parameters that are/is missing a value associated with the biomarker and/or the clinical parameter.
59. The method of any one of embodiments 1-58, wherein the input features comprise at least 20 clinical parameters and at least two biomarkers.
60. The method of any one of embodiments 5-10, wherein the group of input features listed in any one of Tables 1-6 comprises at least one biomarker feature and at least one clinical parameter feature, wherein the value of the one or more biomarker features are divided into two groups, and wherein the values of the one or more clinical parameter features are divided into two groups.
61. The method of embodiment 60, wherein the biomarker features comprise at least one of procalcitonin, C-reactive protein, interleukin 6, pentraxin 3, interleukin 8, interleukin-1 receptor antagonist protein, and vascular cell adhesion molecule 1, wherein a first group of concentration values of each biomarker feature has a lower relative significance than a second group of concentration values of the biomarker feature.
62. The method of embodiments 60 or 61, wherein the clinical parameters comprise at least one of platelets, systolic blood pressure, diastolic blood pressure, albumin, and blood oxygen saturation, and wherein a first group of values of each clinical parameter feature has a higher relative significance than a second group of concentration values of the clinical parameter feature.
63. The method of embodiments 60 or 61, wherein the clinical parameters comprise at least one of respiratory rate, blood urea nitrogen, bilirubin, and creatinine, and wherein a first group of values of each clinical parameter feature has a lower relative significance than a second group of concentration values of the clinical parameter feature.
64. The method of embodiment 60, wherein the biomarker features comprise procalcitonin, wherein a first group of procalcitonin concentration values has a lower relative significance than a second group of procalcitonin concentration values.
65. The method of embodiment 60, wherein the biomarker features comprise C-reactive protein, wherein a first group of C-reactive protein concentration values has a lower relative significance than a second group of C-reactive protein concentration values.
66. The method of embodiment 60, wherein the biomarker features comprise interleukin 6, wherein a first group of interleukin 6 concentration values has a lower relative significance than a second group of interleukin 6 concentration values.
67. The method of embodiment 60, wherein the biomarker features comprise pentraxin 3, wherein a first group of pentraxin 3 concentration values has a lower relative significance than a second group of pentraxin 3 concentration values.
68. The method of embodiment 60, wherein the biomarker features comprise interleukin 8, wherein a first group of interleukin 8 concentration values has a lower relative significance than a second group of interleukin 8 concentration values.
69. The method of embodiment 60, wherein the biomarker features comprise interleukin-1 receptor antagonist protein, wherein a first group of interleukin-1 receptor antagonist protein concentration values has a lower relative significance than a second group of interleukin-1 receptor antagonist protein concentration values.
70. The method of embodiment 60, wherein the biomarker features comprise vascular cell adhesion molecule 1, wherein a first group of vascular cell adhesion molecule 1 concentration values has a lower relative significance than a second group of vascular cell adhesion molecule 1 concentration values.
71. The method of embodiment 60, wherein the biomarker features comprise procalcitonin and C-reactive protein, wherein a first group of procalcitonin concentration values has a lower relative significance than a second group of procalcitonin concentration values, and wherein a first group of C-reactive protein concentration values has a lower relative significance than a second group of C-reactive protein concentration values.
72. The method of embodiment 60, wherein the biomarker features comprise procalcitonin and interleukin 6, wherein a first group of procalcitonin concentration values has a lower relative significance than a second group of procalcitonin concentration values, and wherein a first group of interleukin 6 concentration values has a lower relative significance than a second group of interleukin 6 concentration values.
73. The method of embodiment 60, wherein the biomarker features comprise interleukin 6 and C-reactive protein, wherein a first group of interleukin 6 concentration values has a lower relative significance than a second group of interleukin 6 concentration values, and wherein a first group of C-reactive protein concentration values has a lower relative significance than a second group of C-reactive protein concentration values.
74. The method of embodiment 60, wherein the biomarker features comprise procalcitonin, C-reactive protein, and interleukin 6, wherein a first group of procalcitonin concentration values has a lower relative significance than a second group of procalcitonin concentration values, wherein a first group of C-reactive protein concentration values has a lower relative significance than a second group of C-reactive protein concentration values, and wherein a first group of interleukin 6 concentration values has a lower relative significance than a second group of interleukin 6 concentration values.
75. The method of embodiment 60, wherein the biomarker features comprise pentraxin 3 and interleukin-1 receptor antagonist protein, wherein a first group of pentraxin 3 concentration values has a lower relative significance than a second group of pentraxin 3 concentration values, and wherein a first group of interleukin-1 receptor antagonist protein concentration values has a lower relative significance than a second group of interleukin-1 receptor antagonist protein concentration values.
76. The method of embodiment 60, wherein the biomarker features comprise pentraxin 3 and interleukin 8, wherein a first group of pentraxin 3 concentration values has a lower relative significance than a second group of pentraxin 3 concentration values, and wherein a first group of interleukin 8 concentration values has a lower relative significance than a second group of interleukin 8 concentration values.
77. The method of embodiment 60, wherein the biomarker features comprise procalcitonin and interleukin 8, wherein a first group of procalcitonin concentration values has a lower relative significance than a second group of procalcitonin concentration values, and wherein a first group of interleukin 8 concentration values has a lower relative significance than a second group of interleukin 8 concentration values.
78. The method of embodiment 60, wherein the biomarker features comprise procalcitonin and pentraxin 3, wherein a first group of procalcitonin concentration values has a lower relative significance than a second group of procalcitonin concentration values, and wherein a first group of pentraxin 3 concentration values has a lower relative significance than a second group of pentraxin 3 concentration values.
79. The method of embodiment 60, wherein the biomarker features comprise pentraxin 3 and interleukin 6, wherein a first group of pentraxin 3 concentration values has a lower relative significance than a second group of pentraxin 3 concentration values, and wherein a first group of interleukin 6 concentration values has a lower relative significance than a second group of interleukin 6 concentration values.
80. The method of embodiment 60, wherein the clinical parameter features comprise platelets, wherein a first group of platelets values has a higher relative significance than a second group of platelets values.
81. The method of embodiment 60, wherein the clinical parameter features comprise systolic blood pressure, wherein a first group of systolic blood pressure values has a higher relative significance than a second group of systolic blood pressure values.
82. The method of embodiment 60, wherein the clinical parameter features comprise diastolic blood pressure, wherein a first group of diastolic blood pressure values has a higher relative significance than a second group of diastolic blood pressure values.
83. The method of embodiment 60, wherein the clinical parameter features comprise blood oxygen saturation, wherein a first group of blood oxygen saturation values has a higher relative significance than a second group of blood oxygen saturation values.
84. The method of embodiment 60, wherein the clinical parameter features comprise albumin, wherein a first group of blood oxygen saturation values has a higher relative significance than a second group of albumin values.
85. The method of embodiment 60, wherein the clinical parameter features comprise respiratory rate, wherein a first group of respiratory rate values has a lower relative significance than a second group of respiratory rate values.
86. The method of embodiment 60, wherein the clinical parameter features comprise blood urea nitrogen, wherein a first group of blood urea nitrogen values has a lower relative significance than a second group of blood urea nitrogen values.
87. The method of embodiment 60, wherein the clinical parameter features comprise bilirubin, wherein a first group of bilirubin values has a lower relative significance than a second group of bilirubin values.
88. The method of embodiment 60, wherein the clinical parameter features comprise creatinine, wherein a first group of creatinine values has a lower relative significance than a second group of creatinine values.
89. The method of embodiment 60, wherein the clinical parameter features comprise creatinine and blood urea nitrogen, wherein a first group of creatinine values has a lower relative significance than a second group of creatinine values, and wherein a first group of blood urea nitrogen values has a lower relative significance than a second group of blood urea nitrogen values.
90. The method of embodiment 60, wherein the clinical parameter features comprise platelets, creatinine, and blood urea nitrogen, wherein the clinical parameter features comprise platelets, wherein a first group of platelets values has a higher relative significance than a second group of platelets values, wherein a first group of creatinine values has a lower relative significance than a second group of creatinine values, and wherein a first group of blood urea nitrogen values has a lower relative significance than a second group of blood urea nitrogen values.
91. The method of embodiment 60, wherein the clinical parameter features comprise respiratory rate, creatinine, and blood urea nitrogen, wherein the clinical parameter features comprise platelets, wherein a first group of respiratory rate values has a lower relative significance than a second group of respiratory rate values, wherein a first group of creatinine values has a lower relative significance than a second group of creatinine values, and wherein a first group of blood urea nitrogen values has a lower relative significance than a second group of blood urea nitrogen values.
92. The method of embodiment 60, wherein the clinical parameter features comprise platelets and respiratory rate, wherein the clinical parameter features comprise platelets, wherein a first group of platelets values has a higher relative significance than a second group of platelets values, and wherein a first group of respiratory rate values has a lower relative significance than a second group of respiratory rate values.
93. The method of embodiment 60, wherein the clinical parameter features comprise platelets and systolic blood pressure, wherein the clinical parameter features comprise platelets, wherein a first group of platelets values has a higher relative significance than a second group of platelets values, and wherein a first group of systolic blood pressure values has a higher relative significance than a second group of systolic blood pressure values.
94. The method of embodiment 60, wherein the clinical parameter features comprise platelets, respiratory rate, and systolic blood pressure, wherein the clinical parameter features comprise platelets, wherein a first group of platelets values has a higher relative significance than a second group of platelets values, wherein a first group of respiratory rate values has a lower relative significance than a second group of respiratory rate values, and wherein a first group of systolic blood pressure values has a higher relative significance than a second group of systolic blood pressure values.
95. The method of embodiment 60, wherein the relative significance of one or more features to the predicted score in each of the first group and the second group is a median SHAP value for the feature values within the respective group.
96. The method of embodiment 95, wherein the relative significance of one or more biomarker features to the predicted score in each of the first group and the second group is a median SHAP value for the biomarker feature values within the respective group.
97. The method of embodiment 95, wherein the relative significance of one or more clinical parameter features to the predicted score in each of the first group and the second group is a median SHAP value for the clinical parameter feature values within the respective group.
98. The method of embodiment 60, wherein the relative significance of one or more features to the predicted score in each of the first group and the second group is a mean SHAP value for the feature values within the respective group.
99. The method of embodiment 98, wherein the relative significance of one or more biomarker features to the predicted score in each of the first group and the second group is a mean SHAP value for the biomarker feature values within the respective group.
100. The method of embodiment 98, wherein the relative significance of one or more clinical parameter features to the predicted score in each of the first group and the second group is a mean SHAP value for the biomarker feature values within the respective group.
101. The method of any one of embodiments 61-100, wherein a dividing threshold value between the first group and second group of procalcitonin concentration values is 2.24 log 10 μg/mL.
102. The method of any one of embodiments 61-100, wherein a dividing threshold value between the first group and second group of pentraxin 3 concentration values is 3.98 log 10 μg/mL.
103. The method of any one of embodiments 61-100, wherein a dividing threshold value between the first group and second group of interleukin 8 concentration values is 1.41 log 10 μg/mL.
104. The method of any one of embodiments 61-100, wherein a dividing threshold value between the first group and second group of interleukin-1 receptor antagonist protein concentration values is 3.48 log 10 μg/mL.
105. The method of any one of embodiments 61-100, wherein a dividing threshold value between the first group and second group of interleukin 6 concentration values is 2.07 log 10 μg/mL.
106. The method of any one of embodiments 61-100, wherein a dividing threshold value between the first group and second group of C-Reactive Protein concentration values is 7.63 log 10 μg/mL.
107. The method of any one of embodiments 61-100, wherein a dividing threshold value between the first group and second group of vascular cell adhesion molecule 1 concentration values is 6.17 log 10 μg/mL.
108. The method of any one of embodiments 61-100, wherein a dividing threshold value between the first group and second group of platelets concentration values is 162 10\hat{ }9/L.
109. The method of any one of embodiments 61-100, wherein a dividing threshold value between the first group and second group of systolic blood pressure values is 101 mm Hg.
110. The method of any one of embodiments 61-100, wherein a dividing threshold value between the first group and second group of respiratory rate values is 101 breaths per minute.
111. The method of any one of embodiments 61-100, wherein a dividing threshold value between the first group and second group of blood urea nitrogen values is 24 mg/dL.
112. The method of any one of embodiments 61-100, wherein a dividing threshold value between the first group and second group of bilirubin values is 1.19 mg/dL.
113. The method of any one of embodiments 61-100, wherein a dividing threshold value between the first group and second group of creatinine values is 1.56 mg/dL.
114. The method of any one of embodiments 61-100, wherein a dividing threshold value between the first group and second group of diastolic blood pressure values is 54.5 mm Hg.
115. The method of any one of embodiments 61-100, wherein a dividing threshold value between the first group and second group of blood oxygen saturation values is 91%.
116. The method of any one of embodiments 5-10, wherein the group of input features listed in any one of Tables 1-6 comprises at least one biomarker feature and at least one clinical parameter feature, wherein the value of the one or more biomarker features are divided into at least three groups, and wherein the values of the one or more clinical parameter features are divided into at least three groups.
117. The method of embodiment 116, wherein the biomarker features comprise at least one of procalcitonin, C-reactive protein, interleukin 6, pentraxin 3, interleukin 8, interleukin-1 receptor antagonist protein, and vascular cell adhesion molecule 1, wherein a first group of concentration values of each biomarker feature has a lower relative significance than a second group of concentration values of the biomarker feature, and wherein a third group of concentration values of the biomarker feature has a higher relative significance than both the first group and second group of concentration values.
118. The method of embodiment 118, wherein the clinical parameters comprise at least one of platelets, systolic blood pressure, diastolic blood pressure, albumin, and pulse oximetry, and wherein a first group of values of each clinical parameter feature has a higher relative significance than a second group of concentration values of the clinical parameter feature, and wherein a third group of concentration values of the clinical parameter feature has a lower relative significance than both the first group and second group of clinical parameter values.
119. The method of embodiment 118, wherein the clinical parameters comprise at least one of respiratory rate, blood urea nitrogen, bilirubin, and creatinine, and wherein a first group of values of each clinical parameter feature has a lower relative significance than a second group of concentration values of the clinical parameter feature, and wherein a third group of each clinical parameter feature has a higher relative significance than both the first group and second group of clinical parameter feature values.
120. The method of embodiment 116, wherein the biomarker features comprise procalcitonin, wherein a first group of procalcitonin concentration values has a lower relative significance than a second group of procalcitonin concentration values, and wherein a third group of procalcitonin concentration values has a higher relative significance than both the first group and second group of procalcitonin concentration values.
121. The method of embodiment 116, wherein the biomarker features comprise C-reactive protein, wherein a first group of C-reactive protein concentration values has a lower relative significance than a second group of C-reactive protein concentration values, and wherein a third group of C-reactive protein concentration values has a higher relative significance than both the first group and second group of C-reactive protein concentration values.
122. The method of embodiment 116, wherein the biomarker features comprise interleukin 6, wherein a first group of interleukin 6 concentration values has a lower relative significance than a second group of interleukin 6 concentration values, and wherein a third group of interleukin 6 concentration values has a higher relative significance than both the first group and second group of interleukin 6 concentration values.
123. The method of embodiment 116, wherein the biomarker features comprise pentraxin 3, wherein a first group of pentraxin 3 concentration values has a lower relative significance than a second group of pentraxin 3 concentration values, and wherein a third group of pentraxin 3 concentration values has a higher relative significance than both the first group and second group of pentraxin 3 concentration values.
124. The method of embodiment 116, wherein the biomarker features comprise interleukin 8, wherein a first group of interleukin 8 concentration values has a lower relative significance than a second group of interleukin 8 concentration values, and wherein a third group of interleukin 8 concentration values has a higher relative significance than both the first group and second group of interleukin 8 concentration values.
125. The method of embodiment 116, wherein the biomarker features comprise interleukin-1 receptor antagonist protein, wherein a first group of interleukin-1 receptor antagonist protein concentration values has a lower relative significance than a second group of interleukin-1 receptor antagonist protein concentration values, and wherein a third group of interleukin-1 receptor antagonist protein concentration values has a higher relative significance than both the first group and second group of interleukin-1 receptor antagonist protein concentration values.
126. The method of embodiment 116, wherein the biomarker features comprise vascular cell adhesion molecule 1, wherein a first group of vascular cell adhesion molecule 1 concentration values has a lower relative significance than a second group of vascular cell adhesion molecule 1 concentration values, and wherein a third group of vascular cell adhesion molecule 1 concentration values has a higher relative significance than both the first group and second group of vascular cell adhesion molecule 1 concentration values.
127. The method of embodiment 116, wherein the biomarker features comprise procalcitonin and C-reactive protein, wherein a first group of procalcitonin concentration values has a lower relative significance than a second group of procalcitonin concentration values, and wherein a third group of procalcitonin concentration values has a higher relative significance than both the first group and second group of procalcitonin concentration values, and wherein a first group of C-reactive protein concentration values has a lower relative significance than a second group of C-reactive protein concentration values, and wherein a third group of C-reactive protein concentration values has a higher relative significance than both the first group and second group of C-reactive protein concentration values.
128. The method of embodiment 116, wherein the biomarker features comprise procalcitonin and interleukin 6, wherein a first group of procalcitonin concentration values has a lower relative significance than a second group of procalcitonin concentration values, and wherein a third group of procalcitonin concentration values has a higher relative significance than both the first group and second group of procalcitonin concentration values, and wherein a first group of interleukin 6 concentration values has a lower relative significance than a second group of interleukin 6 concentration values, and wherein a third group of interleukin 6 concentration values has a higher relative significance than both the first group and second group of interleukin 6 concentration values.
129. The method of embodiment 116, wherein the biomarker features comprise C-reactive protein and interleukin 6, wherein a first group of C-reactive protein concentration values has a lower relative significance than a second group of C-reactive protein concentration values, and wherein a third group of C-reactive protein concentration values has a higher relative significance than both the first group and second group of C-reactive protein concentration values, and wherein a first group of interleukin 6 concentration values has a lower relative significance than a second group of interleukin 6 concentration values, and wherein a third group of interleukin 6 concentration values has a higher relative significance than both the first group and second group of interleukin 6 concentration values.
130. The method of embodiment 116, wherein the biomarker features comprise procalcitonin, C-reactive protein, and interleukin 6, wherein a first group of procalcitonin concentration values has a lower relative significance than a second group of procalcitonin concentration values, and wherein a third group of procalcitonin concentration values has a higher relative significance than both the first group and second group of procalcitonin concentration values, wherein a first group of C-reactive protein concentration values has a lower relative significance than a second group of C-reactive protein concentration values, and wherein a third group of C-reactive protein concentration values has a higher relative significance than both the first group and second group of C-reactive protein concentration values, and wherein a first group of interleukin 6 concentration values has a lower relative significance than a second group of interleukin 6 concentration values, and wherein a third group of interleukin 6 concentration values has a higher relative significance than both the first group and second group of interleukin 6 concentration values.
131. The method of embodiment 116, wherein the biomarker features comprise pentraxin 3 and interleukin-1 receptor antagonist protein, wherein a first group of pentraxin 3 concentration values has a lower relative significance than a second group of pentraxin 3 concentration values, and wherein a third group of pentraxin 3 concentration values has a higher relative significance than both the first group and second group of pentraxin 3 concentration values, and wherein a first group of interleukin-1 receptor antagonist protein concentration values has a lower relative significance than a second group of interleukin-1 receptor antagonist protein concentration values, and wherein a third group of interleukin-1 receptor antagonist protein concentration values has a higher relative significance than both the first group and second group of interleukin-1 receptor antagonist protein concentration values.
132. The method of embodiment 116, wherein the biomarker features comprise pentraxin 3 and interleukin 8, wherein a first group of pentraxin 3 concentration values has a lower relative significance than a second group of pentraxin 3 concentration values, and wherein a third group of pentraxin 3 concentration values has a higher relative significance than both the first group and second group of pentraxin 3 concentration values, and wherein a first group of interleukin 8 concentration values has a lower relative significance than a second group of interleukin 8 concentration values, and wherein a third group of interleukin 8 concentration values has a higher relative significance than both the first group and second group of interleukin 8 concentration values.
133. The method of embodiment 116, wherein the biomarker features comprise procalcitonin and interleukin 8, wherein a first group of procalcitonin concentration values has a lower relative significance than a second group of procalcitonin concentration values, and wherein a third group of procalcitonin concentration values has a higher relative significance than both the first group and second group of procalcitonin concentration values, and wherein a first group of interleukin 8 concentration values has a lower relative significance than a second group of interleukin 8 concentration values, and wherein a third group of interleukin 8 concentration values has a higher relative significance than both the first group and second group of interleukin 8 concentration values.
134. The method of embodiment 116, wherein the biomarker features comprise procalcitonin and pentraxin 3, wherein a first group of procalcitonin concentration values has a lower relative significance than a second group of procalcitonin concentration values, and wherein a third group of procalcitonin concentration values has a higher relative significance than both the first group and second group of procalcitonin concentration values, and wherein a first group of pentraxin 3 concentration values has a lower relative significance than a second group of pentraxin 3 concentration values, and wherein a third group of pentraxin 3 concentration values has a higher relative significance than both the first group and second group of pentraxin 3 concentration values.
135. The method of embodiment 116, wherein the biomarker features comprise pentraxin 3 and interleukin 6, wherein a first group of pentraxin 3 concentration values has a lower relative significance than a second group of pentraxin 3 concentration values, and wherein a third group of pentraxin 3 concentration values has a higher relative significance than both the first group and second group of pentraxin 3 concentration values, and wherein a first group of interleukin 6 concentration values has a lower relative significance than a second group of interleukin 6 concentration values, and wherein a third group of interleukin 6 concentration values has a higher relative significance than both the first group and second group of interleukin 6 concentration values.
136. The method of any one of embodiments 116, wherein the clinical parameter features comprise platelets, wherein a first group of platelets values has a higher relative significance than a second group of platelets values, and wherein a third group of platelets values has a lower relative significance than both the first group and second group of platelets values.
137. The method of any one of embodiments 116, wherein the clinical parameter features comprise systolic blood pressure, wherein a first group of systolic blood pressure values has a higher relative significance than a second group of systolic blood pressure values, and wherein a third group of systolic blood pressure values has a lower relative significance than both the first group and second group of systolic blood pressure values.
138. The method of any one of embodiments 116, wherein the clinical parameter features comprise diastolic blood pressure, wherein a first group of diastolic blood pressure values has a higher relative significance than a second group of diastolic blood pressure values, and wherein a third group of diastolic blood pressure values has a lower relative significance than both the first group and second group of diastolic blood pressure values.
139. The method of any one of embodiments 116, wherein the clinical parameter features comprise pulse oximetry, wherein a first group of pulse oximetry values has a higher relative significance than a second group of pulse oximetry values, and wherein a third group of pulse oximetry values has a lower relative significance than both the first group and second group of pulse oximetry values.
140. The method of any one of embodiments 116, wherein the clinical parameter features comprise albumin, wherein a first group of albumin values has a higher relative significance than a second group of albumin values, and wherein a third group of albumin values has a lower relative significance than both the first group and second group of albumin values.
141. The method of any one of embodiments 116, wherein the clinical parameter features comprise respiratory rate, wherein a first group of respiratory rate values has a higher relative significance than a second group of respiratory rate values, and wherein a third group of respiratory rate values has a lower relative significance than both the first group and second group of respiratory rate values.
142. The method of any one of embodiments 116, wherein the clinical parameter features comprise blood urea nitrogen, wherein a first group of blood urea nitrogen values has a higher relative significance than a second group of blood urea nitrogen values, and wherein a third group of blood urea nitrogen values has a lower relative significance than both the first group and second group of blood urea nitrogen values.
143. The method of any one of embodiments 116, wherein the clinical parameter features comprise bilirubin, wherein a first group of bilirubin values has a higher relative significance than a second group of bilirubin values, and wherein a third group of bilirubin values has a lower relative significance than both the first group and second group of bilirubin values.
144. The method of any one of embodiments 116, wherein the clinical parameter features comprise creatinine, wherein a first group of creatinine values has a higher relative significance than a second group of creatinine values, and wherein a third group of creatinine values has a lower relative significance than both the first group and second group of creatinine values.
145. The method of any one of embodiments 116, wherein the clinical parameter features comprise creatinine and blood urea nitrogen, wherein a first group of creatinine values has a higher relative significance than a second group of creatinine values, and wherein a third group of creatinine values has a lower relative significance than both the first group and second group of creatinine values, and wherein a first group of blood urea nitrogen values has a higher relative significance than a second group of blood urea nitrogen values, and wherein a third group of blood urea nitrogen values has a lower relative significance than both the first group and second group of blood urea nitrogen values.
146. The method of any one of embodiments 116, wherein the clinical parameter features comprise platelets, creatinine, and blood urea nitrogen, wherein a first group of platelets values has a higher relative significance than a second group of platelets values, and wherein a third group of platelets values has a lower relative significance than both the first group and second group of platelets values, wherein a first group of creatinine values has a higher relative significance than a second group of creatinine values, and wherein a third group of creatinine values has a lower relative significance than both the first group and second group of creatinine values, and wherein a first group of blood urea nitrogen values has a higher relative significance than a second group of blood urea nitrogen values, and wherein a third group of blood urea nitrogen values has a lower relative significance than both the first group and second group of blood urea nitrogen values.
147. The method of any one of embodiments 116, wherein the clinical parameter features comprise respiratory rate, creatinine, and blood urea nitrogen, wherein a first group of respiratory rate values has a higher relative significance than a second group of respiratory rate values, and wherein a third group of respiratory rate values has a lower relative significance than both the first group and second group of respiratory rate values, wherein a first group of creatinine values has a higher relative significance than a second group of creatinine values, and wherein a third group of creatinine values has a lower relative significance than both the first group and second group of creatinine values, and wherein a first group of blood urea nitrogen values has a higher relative significance than a second group of blood urea nitrogen values, and wherein a third group of blood urea nitrogen values has a lower relative significance than both the first group and second group of blood urea nitrogen values.
148. The method of any one of embodiments 116, wherein the clinical parameter features comprise platelets and respiratory rate, wherein a first group of platelets values has a higher relative significance than a second group of platelets values, and wherein a third group of platelets values has a lower relative significance than both the first group and second group of platelets values, and wherein a first group of respiratory rate values has a higher relative significance than a second group of respiratory rate values, and wherein a third group of respiratory rate values has a lower relative significance than both the first group and second group of respiratory rate values.
149. The method of any one of embodiments 116, wherein the clinical parameter features comprise platelets and systolic blood pressure, wherein a first group of platelets values has a higher relative significance than a second group of platelets values, and wherein a third group of platelets values has a lower relative significance than both the first group and second group of platelets values, and wherein a first group of systolic blood pressure values has a higher relative significance than a second group of systolic blood pressure values, and wherein a third group of systolic blood pressure values has a lower relative significance than both the first group and second group of systolic blood pressure values.
150. The method of any one of embodiments 116, wherein the clinical parameter features comprise platelets, respiratory rate, and systolic blood pressure, wherein a first group of platelets values has a higher relative significance than a second group of platelets values, and wherein a third group of platelets values has a lower relative significance than both the first group and second group of platelets values, wherein a first group of respiratory rate values has a higher relative significance than a second group of respiratory rate values, and wherein a third group of respiratory rate values has a lower relative significance than both the first group and second group of respiratory rate values, and wherein a first group of systolic blood pressure values has a higher relative significance than a second group of systolic blood pressure values, and wherein a third group of systolic blood pressure values has a lower relative significance than both the first group and second group of systolic blood pressure values.
151. The method of embodiment 116, wherein the relative significance of one or more features to the predicted score in each of the first, second, and third groups is a median SHAP value for the feature values within the respective group.
152. The method of embodiment 151, wherein the relative significance of one or more biomarker features to the predicted score in each of the first, second, and third groups is a median SHAP value for the biomarker feature values within the respective group.
153. The method of embodiment 151, wherein the relative significance of one or more clinical parameter features to the predicted score in each of the first, second, and third groups is a median SHAP value for the clinical parameter feature values within the respective group.
154. The method of embodiment 116, wherein the relative significance of one or more features to the predicted score in each of the first, second, and third groups is a mean SHAP value for the feature values within the respective group.
155. The method of embodiment 154, wherein the relative significance of one or more biomarker features to the predicted score in each of the first, second, and third groups is a mean SHAP value for the biomarker feature values within the respective group.
156. The method of embodiment 154, wherein the relative significance of one or more clinical parameter features to the predicted score in each of the first, second, and third groups is a mean SHAP value for the clinical parameter feature values within the respective group.
157. The method of any one of embodiments 116-156, wherein a dividing threshold value between the first group and second group of procalcitonin concentration values is 2.05 log 10 μg/mL, and wherein a second dividing threshold value between the second and third groups of procalcitonin concentration values is 2.85 log 10 μg/mL.
158. The method of any one of embodiments 116-156, wherein a dividing threshold value between the first group and second group of pentraxin 3 concentration values is 3.61 log 10 μg/mL, and wherein a second dividing threshold value between the second and third groups of pentraxin 3 concentration values is 3.98 log 10 μg/mL.
159. The method of any one of embodiments 116-156, wherein a dividing threshold value between the first group and second group of interleukin 8 concentration values is 1.36 log 10 μg/mL, and wherein a second dividing threshold value between the second and third groups of interleukin 8 concentration values is 1.97 log 10 μg/mL.
160. The method of any one of embodiments 116-156, wherein a dividing threshold value between the first group and second group of interleukin-1 receptor antagonist protein concentration values is 3.15 log 10 μg/mL, and wherein a second dividing threshold value between the second and third groups of interleukin-1 receptor antagonist protein concentration values is 3.57 log 10 μg/mL.
161. The method of any one of embodiments 116-156, wherein a dividing threshold value between the first group and second group of interleukin 6 concentration values is 3.15 log 10 μg/mL, and wherein a second dividing threshold value between the second and third groups of interleukin 6 concentration values is 3.57 log 10 μg/mL.
162. The method of any one of embodiments 116-156, wherein a dividing threshold value between the first group and second group of C-Reactive Protein concentration values is 7.31 log 10 μg/mL, and wherein a second dividing threshold value between the second and third groups of C-Reactive Protein concentration values is 7.91 log 10 μg/mL.
163. The method of any one of embodiments 116-156, wherein a dividing threshold value between the first group and second group of vascular cell adhesion molecule 1 concentration values is 6.05 log 10 μg/mL, and wherein a second dividing threshold value between the second and third groups of vascular cell adhesion molecule 1 concentration values is 6.17 log 10 μg/mL.
164. The method of any one of embodiments 116-156, wherein a dividing threshold value between the first group and second group of platelets concentration values is 139 10\hat{ }9/L, and wherein a second dividing threshold value between the second and third groups of platelets concentration values is 188 10\hat{ }9/L.
165. The method of any one of embodiments 116-156, wherein a dividing threshold value between the first group and second group of systolic blood pressure values is 91 mm Hg, and wherein a second dividing threshold value between the second and third groups of systolic blood pressure values is 111 mm Hg.
166. The method of any one of embodiments 116-156, wherein a dividing threshold value between the first group and second group of respiratory rate values is 23.5 breaths per minute, and wherein a second dividing threshold value between the second and third groups of respiratory rate values is 28 breaths per minute.
167. The method of any one of embodiments 116-156, wherein a dividing threshold value between the first group and second group of blood urea nitrogen values is 21 mg/dL, and wherein a second dividing threshold value between the second and third groups of blood urea nitrogen values is 41 mg/dL.
168. The method of any one of embodiments 116-156, wherein a dividing threshold value between the first group and second group of bilirubin values is 0.88 mg/dL, and wherein a second dividing threshold value between the second and third groups of bilirubin values is 1.5 mg/dL.
169. The method of any one of embodiments 116-156, wherein a dividing threshold value between the first group and second group of creatinine values is 1.15 mg/dL, and wherein a second dividing threshold value between the second and third groups of creatinine values is 1.73 mg/dL.
170. The method of any one of embodiments 116-156, wherein a dividing threshold value between the first group and second group of diastolic blood pressure values is 52 mm Hg, and wherein a second dividing threshold value between the second and third groups of diastolic blood pressure values is 66 mm Hg.
171. The method of any one of embodiments 116-156, wherein a dividing threshold value between the first group and second group of blood oxygen saturation values is 81 mm Hg, and wherein a second dividing threshold value between the second and third groups of blood oxygen saturation values is 92.5 mm Hg.
172. A method, wherein generating the diagnosis output based on the predicted score from the method of any one of embodiments 4-171 classifies the patient as having sepsis, or being at risk for developing sepsis within a predetermined time-span, the method further comprising:
administering to the individual a therapeutically effective amount of the treatment for sepsis.
173. The method of embodiment 172, wherein the treatment comprises at least one of an antibiotic or an intravenous fluid based on the generated diagnosis output.
174. A method of treating an individual for sepsis having a score based on multiple input features that indicates a probability of the patient having or developing sepsis based on the method of any one of embodiments 1-171, said treating comprising administering to the individual an effective amount of one or more sepsis therapies.
175. A method, comprising measuring multiple input features in Table 8, Table 9, Table 10, Table 11, Table 12, or Table 13 of an individual and/or from a sample from an individual, optionally wherein measuring of at least a first input feature and at least a second input feature is successive, and the measuring of the second input feature is predicated upon the outcome of the measuring of the first input feature.
176. The method of embodiment 175, comprising measuring the majority of multiple input features in Table 8 of the individual.
177. The method of embodiment 175 or 176, wherein the individual is suspected of having sepsis, suspected of developing sepsis, or is at risk for developing sepsis, or wherein the individual has one or more symptoms of sepsis.
178. The method of any one of embodiments 175-177, wherein the individual is suspected of developing sepsis within 24 hours of the measuring of the individual.
179. The method of any one of embodiments 175-178, wherein the sample is blood, urine, tissue, skin, saliva, phlegm, and/or mucus.
180. The method of any one of embodiments 175-179, wherein the individual has an infection.
181. The method of embodiment 180, wherein the infection is viral, bacterial, fungal, or prion.
182. The method of embodiment 180 or 181, wherein the infection is SARS-CoV2, influenza, meningitis, pneumonia, tuberculosis, or hepatitis.
183. The method of any one of embodiments 175-182, wherein the individual is greater than about 65 years of age, less than about one years of age, is immunocompromised, has a chronic medical condition, has had a recent severe illness or hospitalization, and/or has previously had sepsis.
184. The method of embodiment 183, wherein the chronic medical condition is diabetes, lung disease, cancer, heart disease, hepatitis, or kidney disease.
185. A method, comprising measuring 1, 2, 3, 4, 5, 6, 7, 8, 9, 10, 11, 12, 13, 14, 15, 16, 17, 18, 19, 20, 21, or more input features in any one or more of Tables 8, 9, 10, 11, 12, or 13 of an individual.
186. The method of embodiment 185, wherein the individual has one or more symptoms of sepsis.
187. The method of embodiment 185 or 186, wherein the individual is suspected of having sepsis, suspected of developing sepsis, or is at risk for developing sepsis.
188. The method of any one of embodiments 185-187, wherein the individual is suspected of developing sepsis within 24 hours of the measuring in a sample obtained from the individual.
189. The method of any one of embodiments 185-188, wherein the individual has an infection.
190. The method of embodiment 189, wherein the infection is viral, bacterial, fungal, or prion.
191. The method of embodiment 189 or 190, wherein the infection is SARS-CoV2, influenza, meningitis, pneumonia, tuberculosis, or hepatitis.
192. The method of any one of embodiments 185-191, wherein the individual is greater than about 65 years of age, less than about one years of age, is immunocompromised, has a chronic medical condition, has had a recent severe illness or hospitalization, or has previously had sepsis.
193. The method of embodiment 192, wherein the chronic medical condition is diabetes, lung disease, cancer, heart disease, or kidney disease.
194. The method of any one of embodiments 1-171, wherein a plurality of input features includes at least one input feature of a first group, at least one input feature of a second group, and at least one input feature of a third group.
195. The method of embodiment 194,
wherein the first group comprises the input features selected from the group consisting of: age, sex at birth, race, ethnicity, past medical history of the patient, current complaints or symptoms of the patient, neurological assessments, clinical decision support alerts, clinician or other chart notes, diagnosis codes, procedures performed on the patient, current medications of the patient, interventions, patient care setting, medical imaging data or assessments, electrograms, endoscopic tests, systolic blood pressure, diastolic blood pressure, body temperature, respiratory rate, heart rate, blood oxygen saturation, a fraction of inspired oxygen, and a combination thereof;
wherein the second group comprises the input features selected from the group consisting of: white blood cell count, lymphocyte count, neutrophil count, platelet count, creatinine, blood urea nitrogen, potassium, chloride, total carbon dioxide, sodium, albumin, bilirubin, lactate, glucose, calcium, hematocrit, hemoglobin concentration, lab-derived biomarkers, clinical severity measures and composites, biopsies data, microorganism test data, and a combination thereof; and
wherein the third group comprises the input features selected from the group consisting of: procalcitonin, C-reactive protein, protein biomarkers, genomic biomarkers, gene expression or transcriptomic biomarkers, composite biomarker scores, and a combination thereof.
196. The method of embodiment 194 or 195, wherein the first group comprises the input features selected from the group consisting of:
vitals (e.g., temperature, heart rate, blood pressure, respiratory rate, oxygen saturation);
demographics (e.g., age, sex, race, ethnicity);
past medical history;
current complains or symptoms;
neurological assessments (Glasgow Coma Scale, Full Outline of Unresponsiveness (FOUR);
clinical decision support alerts;
clinician or other chart notes;
diagnosis codes (e.g., ICD-10);
procedures (including CPT codes);
current medications (including NDC codes);
interventions;
patient care setting (ICU, ED, hospital floor, outpatient clinic, etc.);
imaging (e.g., X-ray, CT scan, MRI, fMRI, ultrasound);
electrograms (e.g., EEG, EKG);
   endoscopic tests; and
a combination thereof.
197. The method of any one of embodiments 194-196, wherein the second group comprises the input features selected from the group consisting of:
labs;
lab-derived biomarkers (e.g., monocyte distribution width, Cytovale IntelliSep);
clinical severity measures and composites (e.g., Sequential Organ Failure Assessment (SOFA) score, SOFA component scores, Charlson Comorbidity Index, Acute Physiology and Chronic Health Evaluation (APACHE) I and II);
biopsies;
microorganism tests (cultures, PCR tests, antigen tests);
and a combination thereof.
198. The method of any one of embodiments 194-196, wherein the third group comprises the input features selected from the group consisting of:
protein biomarkers;
genomic biomarkers;
gene expression or transcriptomic biomarkers;
composite biomarker scores; and
a combination thereof.
199. The method of embodiment 198, wherein the protein markers are selected from the group consisting of Angiopoietin-1, Angiopoietin-2, C-Reactive Protein, Cystatin C, D-Dimer, E-Selectin, Fractalkine, FLT3-Ligand, GCSF, GDF15, GMCSF, Granzyme-B, IFN-alpha, IFN-gamma, IL1-beta, IL1-RA, IL-2, IL-4, IL-6, IL-7, IL-8, IL-10, IL-15, IP-10, lactate dehydrogenase (LDH), lipopolysaccharide-binding protein (LBP), Leptin, MCP1, MIP1-alpha, MIP1-beta, MIP3-alpha, NGAL, Pancreatic Stone Protein, PDL-1, Pentraxin-3, Procalcitonin, Protein C, S100b, TGF-alpha, Thromodulin, Tissue Factor, TNF-alpha, TRAIL, TREM-1, Troponin, VCAM-1, VEG-F, and a combination thereof.
200. The method of embodiment 198 or 199, wherein the gene expression or transcriptomic biomarkers are selected from the group consisting of CEACAM4, LAMP1, PLAC8, PLA2G7, CEACAMI, ZDHHC19, C9orf95, GNA15, BATF, C3AR1, KIAA1370, TGFBI, MTCH1, RPGRIP1, HLA-DPB1, Nuclear factor-kappa B (NF-kB), Heme oxygenase-1 (HO-1), FCGR2C, CRP, IL-6, IL-10, IL-12, TNF, LY6E, LAMP3, ISG15, USP18, RSAD2, IFI44L, NCOA7, CMPK2, IFI44, BATF2, GBP4, IFIH1, GMPR, CXCL10, IFI27, ISG15, CCL2, OAS3, P2RY14, GCH1, CD274, IDO1, ARL4A, BPGM, TNFAIP3, TNFAIP6, IL1R1, PROK2, RGS1, CXCR6, RTP4, TLR3, FZD5, KCNJ2, IRAK2, RGS16, CXCL8, OLR1, CCRL2, SPP1, ST3GAL4, CST7, IL1R2, IL4R, GALM, CTLA4, SOCS1, BCL2L1, TGM2, SLC39A8, CA2, PNP, GATA1, SLC1A5, RGS16, CD44, PDGFC, CSF2RA, CXCL1, CSF3R, SOCS3, IL18R1, PIM1, LEPR, IL1B, CSF2RB, ITGB3, STAT1, FAS, EFNA1, IER3, EETS2, SLC2A3, BCL2A1, PFKFB3, CEBPB, BIRC2, ATF3, TUBB2A, G0S2, MERTK, BIK, QSOX1, PYGL, TGFA, P4HA2, LDHA, IRS2, TSPO, HGF, GADD45A, TGFBR3, CD38, PRF1, FASLG, TIMP3, ANKH, LGALS3, PPP2R5B, GPX1, BNIP3L, BCL2L1, KLF7, FOSL2, ADM, MXI1, SELENBP1, PGF, FOXO3, NEDD4L, SLC2A1, SIAH2, MMP9, CXCR3, CD8A, IFNG, CD8B, JAK2, CCL4, ICAM1, WARS1, KRT1, GPR65, DYRK3, ACHE, ARG1, LCN2, LTF, OLFM4, HLA-DMB, YKT6, PDE4B, TWISTNB, BTN2A2, ZBTB33, PSMB9, CAMK4, TMEM19, SLC12A7, TP53BP1, PLEKHO1, SLC25A22, FRS2, CD24, STX1A, KCNMB4, CRISP2, HTRA1, PPL, RHBDF2, ZCCHC4, DDX6, SENP5, RAPGEF1, DTX2, RELB, DYRK2, CCNB1IP1, TDRD9, ZAP70, ARL14EP, MDC1, ADGRE3, and a combination thereof.
201. The method of any one of embodiments 198-200, wherein the composite biomarker scores are selected from the group consisting of SeptiCyte LAB, Septicyte RAPID, Inflammatix TruVerity, and a combination thereof.

1.-201. (canceled)

202. A method of generating an indicator of risk for sepsis, the method comprising:
receiving at least one input feature corresponding to a patient; and
analyzing the at least one input feature using a machine learning model to generate a predicted score that indicates a probability of the patient having or developing sepsis within a predetermined time-span based on the at least one input feature, wherein the at least one input feature is associated with sepsis.

203. The method of claim 202, wherein the at least one input feature comprises at least one of a clinical parameter or a biomarker, wherein the clinical parameter and/or the biomarker is/are selected from a group of input features listed in Table 8, Table 9, Table 10, Table 11, Table 12, and/or Table 13.

204. The method of claim 202, further comprising:
determining that the predicted score falls within one of four categories; and
generating, based on a category in which the predicted score falls, a diagnosis output that includes that the patient has a low, medium, high, or very high probability of having or developing sepsis within the predetermined time-span.

205. The method of claim 202, further comprising:
calibrating the predicted score using a calibration model to generate a calibrated score; and
generating a risk category as a diagnosis output in view of the calibrated score.

206. The method of claim 205, wherein generating the diagnosis output comprises:
determining that the calibrated score falls within one of four risk categories; and
generating, based on the category in which the calibrated score falls, the risk category that includes that the patient has a low, medium, high, or very high probability of having or developing sepsis within the predetermined time-span.

207. The method of claim 202, further comprising:
training the machine learning model using training data that comprise a plurality of biomarkers and health profiles for a plurality of patients and a plurality of subject diagnoses for the plurality of patients.

208. The method of claim 202, further comprising:
generating a diagnosis output based on the predicted score, wherein the diagnosis output is an automatically derived label based on the predicted score generated by the machine learning model.

209. The method of claim 202, further comprising:
generating a diagnosis output based on the predicted score, wherein the diagnosis output is adjudicated by a physician in view of the predicted score generated by the machine learning model.

210. The method of claim 203, further comprising:
identifying and imputing a value for one input feature from the at least one of the clinical parameter and/or the biomarker that is missing a value.

211. The method of claim 202, wherein one input feature of the at least one input feature has a missing value, the method further comprising:
using a trigger logic decision making module to determine whether to perform the analyzing without the missing value or the one input feature to generate the predicted score.

212. The method of claim 202, wherein one input feature of the at least one input feature has a missing value, the method further comprising:
imputing the missing value for the one input feature to generate an imputed value for the missing input feature;
further analyzing the at least one input feature and the imputed value to generate an updated predicted score that indicates an updated probability of the patient having or developing sepsis within the predetermined time-span; and
generating a diagnosis output based on the updated predicted score.

213. The method of claim 212, wherein the imputing of the missing value is performed via a pre-trained template that is trained using training data, the training data comprising input features that include a plurality of clinical parameters and a plurality of biomarkers for a plurality of patients, and a plurality of subject diagnoses for the plurality of patients.

214. The method of claim 212, wherein the one input feature that has the missing value is designated as a feature whose value is measured or obtained outside of a prescribed time window for data acceptable for use in generating the predicted score.

215. The method of claim 202, wherein the at least one input feature comprises a plurality of biomarkers, the method further comprising:
identifying and imputing a biomarker from the plurality of biomarkers that is missing a value associated with the biomarker.

216. A method, wherein generating the diagnosis output based on the predicted score from the method of claim 204 classifies the patient as having sepsis, or being at risk for developing sepsis within the predetermined time-span, the method further comprising:
administering to the patient a therapeutically effective amount of treatment for sepsis.

217. The method of claim 216, wherein the treatment comprises at least one of an antibiotic or an intravenous fluid based on the diagnosis output.

218. The method of claim 202, further comprising:
generating a diagnosis output based on the predicted score,
wherein the diagnosis output classifies the patient as having sepsis, or being at risk for developing sepsis within the predetermined time-span; and
administering to the patient a therapeutically effective amount of treatment for sepsis.

219. The method of claim 218, wherein the treatment comprises at least one of an antibiotic or an intravenous fluid based on the diagnosis output.

220. A method of treating an individual for sepsis having a score based on multiple input features that indicates a probability of the individual having or developing sepsis based on the method of claim 202, said treating comprising administering to the individual an effective amount of one or more sepsis therapies.

221. A method, comprising:
measuring multiple input features in Table 8, Table 9, Table 10, Table 11, Table 12, or Table 13 of an individual and/or from a sample from an individual, wherein measuring of at least a first input feature and at least a second input feature is successive, and the measuring of the second input feature is predicated upon the outcome of the measuring of the first input feature.
